(19) United States
(12) Patent Application Publication
(10) Pub. No.: US 2013/0046013 A1
(43) Pub. Date: Feb. 21, 2013

(54) SALICYLATE FATTY ACID DERIVATIVES

(71) Applicants: Ragnar Hovland, Nesoddtangen (NO); Tore Skæret, Oslo (NO); Jenny Rosman, Marstrand (SE)

(72) Inventors: Ragnar Hovland, Nesoddtangen (NO); Tore Skæret, Oslo (NO); Jenny Rosman, Marstrand (SE)

(21) Appl. No.: 13/574,132

(22) PCT Filed: Jan. 20, 2011
(86) PCT No.: PCT/IB2011/000250

§ 371 Date: Nov. 8, 2012

Related U.S. Application Data
(60) Provisional application No. 61/296,717, filed on Jan. 20, 2010.

(51) Int. Cl. C07C 69/734; C07C 59/60; A61K 31/19; C07C 69/78; A61K 31/235; C07C 255/30; A61K 31/275; A61P 29/00; A61P 1/00; A61P 9/10; A61P 3/10; A61P 5/50; A61P 3/06; A61P 3/00; A61K 31/22

(52) U.S. Cl. 514/528; 560/187; 514/546; 562/588; 514/557; 560/60; 514/533; 558/445

(57) ABSTRACT

Fatty acid conjugates of salicylate derivatives and compositions thereof are disclosed. Further disclosed are methods for treating various diseases comprising the administration of an effective amount of at least one compound according to the present disclosure.

This application claims priority to U.S. Provisional Application No. 61/296,717, filed on Jan. 20, 2010, which is incorporated by reference herein in its entirety.

The present disclosure generally relates to fatty acid conjugates of salicylate derivatives, and compositions and methods of use thereof. The compositions presently disclosed may comprise an effective amount of a fatty acid conjugate of a salicylate derivative. Also disclosed are methods for treating and/or preventing an inflammatory disease including, e.g., inflammation and/or inflammatory bowel disease (IBD), dyslipidemia including mixed dyslipidemia and/or hypertriglyceridemia, elevated blood lipids including triglycerides and/or cholesterol, metabolic syndrome, peripheral insulin resistance, diabetes, and atherosclerosis; a method for lowering non-HDL cholesterol; and a method for raising HDL-cholesterol comprising the administration of an effective amount of at least one compound according to the present disclosure.

### BACKGROUND

Obesity, occurring at epidemic rates worldwide, is a major risk factor for diabetes and cardiovascular disease. Thus, there is an urgent need for effective interventions to prevent diabetes in obese populations. The importance of lifestyle modification in obesity and diabetes is well recognized. However, disappointing long-term results of these treatments have led to increased interest in pharmaceutical intervention. Obesity and high-fat western diets activate inflammatory processes, which promote development of insulin resistance. Thus, targeting the inflammatory pathway is a novel pharmacologic intervention for diabetes prevention and treatment.

According to World Health Organization (WHO) estimates, more than 220 million people worldwide have diabetes. Diabetes is known to be a significant cause to the development of Cardiovascular Disease (CVD). In 2005 17.5 million people died of CVD. This is 30 percent of all deaths globally and represents the single leading cause of death.

The prevalence of Diabetes type II is increasing rapidly world wide and most people with diabetes will die or be disabled as a consequence of diabetes related complications. It is generally believed that individuals with diabetes have an approximately three-fold increase in the risk of fatal coronary events compared to those without diabetes.

Impaired glucose tolerance (IGT) and impaired fasting glycaemia (IFG) are intermediate conditions in the transition between normality and diabetes. People with IGT or IFG are at high risk of progressing to type 2 diabetes, although this is not inevitable.

There is increasing evidence in explaining the diabetes development among the obese population as being an inflammatory process. Accordingly, anti-inflammatory treatment may be considered as one of several methods to prevent diabetic related diseases.

Salicylates are among the most commonly used nonsteroidal anti-inflammatory drugs. The benefits of salicylates for treatment of diabetes have long been recognized. High doses of the salicylate aspirin (4-7 g/day) improve fasting and postprandial hyperglycemia in patients with diabetes. In recent studies, the hypoglycemic actions of salicylates have been reinvestigated, and the molecular target was identified to be the IκB kinase complex β (IKKβ)/nuclear factor κB (NF-κB) pathway, a central integrator of proinflammatory signals. However, the therapeutic potential of high-dose aspirin is limited by bleeding risk. Salsalate, a dimer of salicylic acid, has an established safety profile after decades of use for rheumatic pain. As a nonacetylated salicylate, salsalate is an equipotent inhibitor of NF-κB but has a lower bleeding risk than aspirin.

Results from a newly published study demonstrate that salsalate reduces glycemia and may improve inflammatory cardiovascular risk indexes in overweight individuals (Fleischman, 2008). These data support the hypothesis that subacute-chronic inflammation contributes to the pathogenesis of obesity-related dysglycemia and that targeting inflammation may provide a therapeutic route for diabetes prevention. Further, another published study demonstrates that salsalate improves in vivo glucose and lipid homeostasis, and support targeting of inflammation and NF-κB as a therapeutic approach in type 2 diabetes (Goldfine, 2008).

Inflammation also participates in the pathogenesis of insulin resistance, type 2 diabetes (T2D), and cardiovascular disease (CVD). Weight gain and obesity are accompanied by activation of at least two inflammatory pathways in adipose tissue and liver, the stress kinase JNK6,7 and the transcription factor NF-κB, which increases the production of proinflammatory cytokines and chemokines (e.g., TNF-α, IL-6, IL-1β, resistin, and MCP-1) and promotes the recruitment of macrophages to adipose tissue. Inflammatory mediators induce insulin resistance locally in fat and liver, and systemically in skeletal muscle. The subacute chronic inflammation of obesity may therefore provide pharmacological targets for intervention (Goldfine, 2008).

While a 2-week course of high-dose (approximately 7 g/d) aspirin reduces glucose and lipid levels and improves insulin sensitivity in patients with diabetes, prolonged exposure to such high doses of aspirin would have unacceptable side effects, especially potentially serious gastrointestinal bleeding. Nonacetylated salicylates do not modify Cox enzymes, inhibit platelets, or prolong bleeding time, and are therefore not associated with increased bleeding risk. Salsalate (Disalsid™), a dimeric prodrug comprising two esterified salicylate moieties, is used to treat patients with rheumatologic conditions. Salsalate is advantageous over sodium salicylate because it is insoluble at the acid pH of the stomach and passes suspended but undissolved into the small intestine, sparing the gastric mucosa direct contact. Blood salicylate levels are nonetheless comparable to those following administration of sodium salicylate. Furthermore, salsalate is generic and inexpensive, so established safety and efficacy in diabetes would have potential health-economic benefit worldwide. In proof-of-concept studies, we assessed the effects of targeting inflammation with salsalate to lower glycemia in patients with type 2 diabetes.

An ongoing study, The Impact of Reducing Inflammation on Vascular Function in the Metabolic Syndrome (ClinicalTrials.gov Identifier: NCT00762827), is set up to test the hypothesis that reductions in intracellular inflammation will restore insulin-mediated and endothelium-dependent vasodilation in subjects with the metabolic syndrome.

The TINSAL-T2D-II (ClinicalTrials.gov Identifier: NCT00799643) is an ongoing study where the primary objective is to determine whether salicylates represent a new pharmacological option for diabetes management. The study is conducted in two stages. The primary objective of the first stage was to select a dose of salsalate that was both well-tolerated and demonstrated a trend toward improvement in glycemic control. The primary objective of Stage II of the study is to evaluate:

the effects of salsalate on glycemic control in diabetes;

the tolerability of salsalate use in patients with type 2 diabetes (T2D);

the effects of salsalate on measures of inflammation, the metabolic syndrome, and cardiac risk.

Another TINSAL study is the TINSAL-CVD (ClinicalTrials.gov Identifier: NCT00624923) where the researchers evaluate the effects of targeting inflammation using salsalate, compared to placebo, on coronary artery plaque volume assessed by multi-detector computed tomographic angiography (MDCTA). No results from the TINSAL studies are yet published.

The anti-inflammatory effects of omega-3 fatty acids have been widely studied with positive results for several chronic inflammatory diseases. TNFα and IL-6 are cytokines that increase dramatically during inflammatory processes and are commonly measured as markers of inflammation. Greater intake of omega-3 PUFA has been shown to associate strongly with lower levels of circulating TNFα and IL-6 as well as with increased levels of markers of anti-inflammation, including the well-characterized anti-inflammatory cytokine IL-10 (Ferruccci et al, 2006). Further, animal models of colitis indicate that fish oil decreases colonic damage and inflammation, weight loss, and mortality.

EPA and DHA have effects on diverse physiological processes impacting normal health and chronic disease, such as the regulation of plasma lipid levels, cardiovascular and immune function, insulin action and neural development and visual function. Firm evidence exist for their beneficial role in the prevention and management of coronary heart disease, dyslipidemias, type 2 diabetes, insulin, resistance, and hypertension (Simonopoulos 1999; Geleijnse 2002; Storlien 1998).

Due to their limited stability in vivo and their lack of biological specificity, PUFAs have not achieved widespread use as therapeutic agents. Chemical modifications of the n-3 polyunsaturated fatty acids have been performed by several research groups in order to change or increase their metabolic effects.

For example, the hypolipidemic effects of EPA was potentiated by introducing methyl or ethyl in α- or β-position of EPA. (Vaagenes 1999). The compounds also reduced plasma free fatty acid while EPA EE had no effect.

Alpha-methyl EPA has been shown to be a stronger inhibitor of platelet aggregation than EPA, both in vitro (Larsen 1998) and in vivo (Willumsen 1998).

Several polyunsaturated fatty acid derivatives with sulfur or oxygen in 3-position have been prepared (Flock et al, Acta Chemica Scand., 1999, 53, 436). Methyl (all-Z)-3-thia-6,9,12,15-octadecatetraenoate was tested in a Wistar rat model, and the effects were compared to the effects of TTA. The results suggest that both the saturated and the unsaturated fatty acids lowered plasma triglycerides to a similar extent (Willumsen et al, J. Lipid Mediators Cell Signalling, 1997, 17, 115).

Several research groups have prepared unsaturated fatty acids with oxygen incorporated in the n-position (Flock, S et al, Acta Chemica Scandinavica, 1999: 53, 436, Pitt, M J, et al, Synthesis, 1997, 1240-42).

### SUMMARY

Conjugates between salicylate derivatives and omega-3 fatty acids like EPA and DHA have previously been proposed for treating inflammatory conditions. The activity of these compounds is reported to be greater than the sum of the two components taken in combination (WO 2010/006085). The present disclosure enhances this effect by introducing fatty acid derivatives into the conjugate that are more potent than natural omega-3 fatty acids, such as EPA and DHA.

The present disclosure relates to fatty acid conjugates of salicylate derivatives, compositions such as pharmaceutical compositions comprising fatty acid conjugates of salicylate derivatives and methods for treating or preventing an inflammatory disease, including inflammation, inflammatory bowel disease (IBD), dyslipidemia including mixed dyslipidemia and hypertriglyceridemia, elevated blood lipids, including triglycerides and cholesterol, metabolic syndrome, peripheral insulin resistance, diabetes, atherosclerosis, a method for lowering non-HDL cholesterol, and a method for raising HDL-cholesterol comprising the administration of an effective amount of at least one compound according to the present disclosure.

Accordingly, in one embodiment, compounds of Formula I are disclosed:

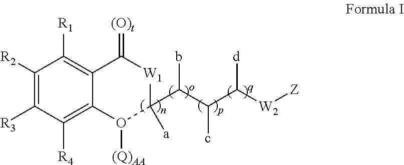

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₁ and W₂ are each independently a bond, O, or —N(R)—, or when W₁ and W₂ are both NH, then both W₁ and W₂ can be taken together to form a piperidine moiety;

[symbol] represents an optional bond that when present requires that AA is 0;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

each Z is H or

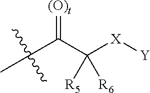

with the proviso that there is at least one

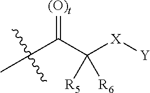

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

Q is C(O)CH₃, Z,

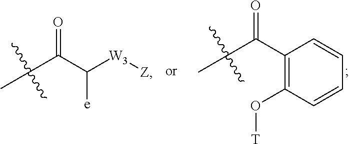

e is H or any one of the side chains of naturally occurring amino acids;

W₃ is a bond, —O—, or —N(R)—;

R is H or C₁-C₃ alkyl;

AA is 0 or 1; and

T is H, C(O)CH₃, or Z.

In another aspect, compounds of the Formula Ia are disclosed:

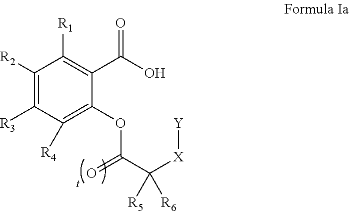

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Ib are disclosed:

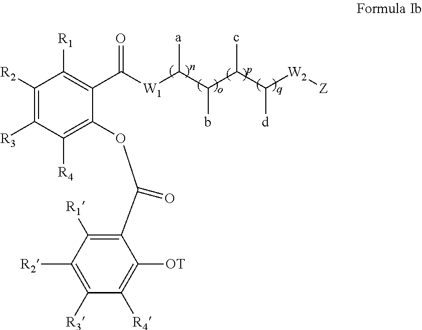

or a pharmaceutically acceptable salt, hydrate, solvate, prodrug, enantiomer, or stereoisomer thereof; wherein

R₁, R₂, R₃, R₄, R₁′, R₂′, R₃′, and R₄′ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₁ and W₂ are each independently a bond, O, or —N(R)—, wherein R is H or C₁-C₃ alkyl, or when W₁ and W₂ are both NH, then both W₁ and W₂ can be taken together to form a piperidine moiety;

a and c are each independently H, CH₃, —OCH₃, —OCH₂CH₃, or C(O)OH;

b is H, CH₃, C(O)OH, or O—Z;

d is H or C(O)OH;

each of n, o, p, and q is independently 0 or 1;

and

T is H, C(O)CH₃, or Z,

wherein each Z is H or

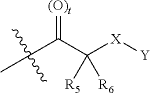

with the proviso that there is at least one

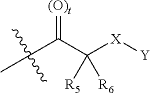

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Ic are disclosed:

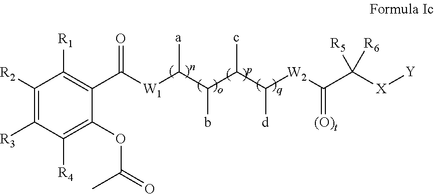

and pharmaceutically acceptable salts, hydrates, solvates, enantiomers, and stereoisomers thereof, wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₁ and W₂ are each independently a bond, O, or —N(R)—, wherein R is H or C₁-C₃ alkyl, or when W₁ and W₂ are both NH, then both W₁ and W₂ can be taken together to form a piperidine moiety;

a and c are each independently H, CH₃, —OCH₃, —OCH₂CH₃, or C(O)OH;

b is H, CH₃, C(O)OH, or O—Z;

d is H or C(O)OH;

n, o, p, and q are each independently 0 or 1;

Z is H, or

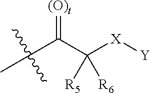

with the proviso that there is at least one

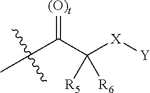

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Id are disclosed:

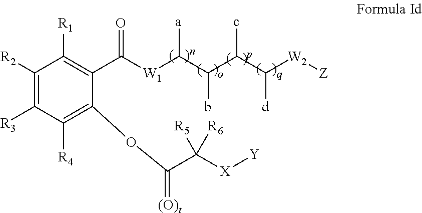

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₁ and W₂ are each independently a bond, O, or —N(R)—, wherein R is H or C₁-C₃ alkyl, or when W₁ and W₂ are both NH, then both W₁ and W₂ can be taken together to form a piperidine moiety;

a and c are each independently H, CH₃, —OCH₃, —OCH₂CH₃, or C(O)OH,

b is H, CH₃, C(O)OH, or O—Z;

d is H or C(O)OH;

n, o, p, and q are each independently 0 or 1;

Z is H, or

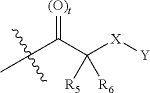

with the proviso that there is at least one

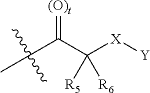

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Ie are disclosed:

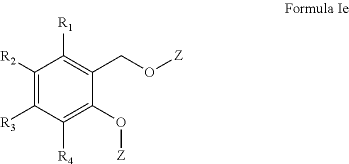

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

each Z is independently H, —C(O)CH₃ or

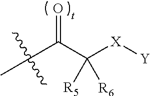

with the proviso that there is at least one

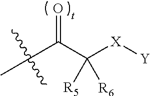

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds in the compound.

In another aspect, compounds of the Formula If are disclosed:

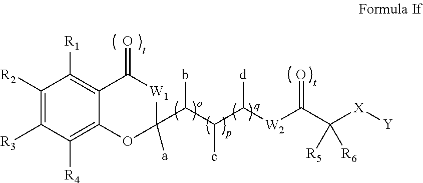

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₂ is a bond, O, or —N(R)—;

a and c are each independently H, CH₃, —OCH₃, —OCH₂CH₃, or C(O)OH;

b is H, CH₃, C(O)OH, or O—Z;

d is H or C(O)OH;

Z is H, or

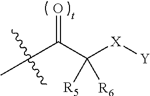

with the proviso that there is at least one

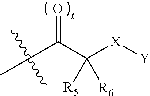

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

W₁ is O or —N(R)—, wherein R is H or C₁-C₃ alkyl; and

each o, p, and q is independently 0 or 1.

In another embodiment, compounds of the Formula Ig are disclosed:

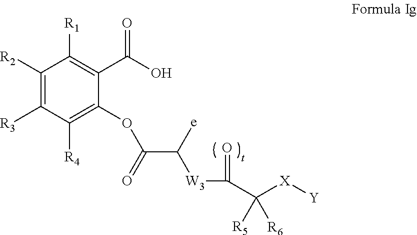

or a pharmaceutically acceptable salt, hydrate, solvate, prodrug, enantiomer, or stereoisomer thereof; wherein

R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;

W₃ is a bond, O, or —N(R)—;

R is H or C₁-C₃ alkyl;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds; and

e is H or any one of the side chains of naturally occurring amino acids.

In another embodiment, compounds of Formula II are disclosed:

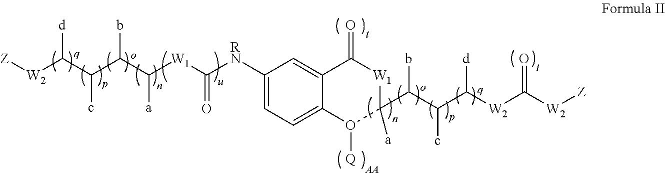

or a pharmaceutically acceptable salt, hydrate, solvate, prodrug, enantiomer, or stereoisomer thereof;

wherein each W₁ and W₂ are independently a bond, O, or —N(R)—, or when W₁ and W₂ are both NH, then both W₁ and W₂ can be taken together to form a piperidine moiety;

[symbol] represents an optional bond that when present requires that AA is 0;

each a and c are independently H, CH₃, —OCH₃, —OCH₂CH₃, or C(O)OH;

each b is H, CH₃, C(O)OH, or O—Z;

each d is H or C(O)OH;

each n, o, p, and q is independently 0 or 1;

each Z is H or

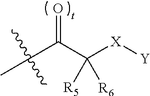

with the proviso that there is at least one

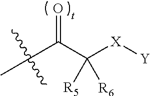

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

u is 0 or 1;

Q is H, C(O)CH₃, Z,

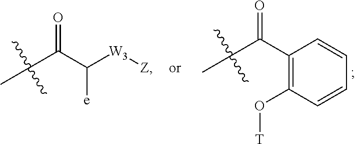

e is H or any one of the side chains of naturally occurring amino acids;

W₃ is a bond, —O—, or —N(R)—;

R is H or C₁-C₃ alkyl;

AA is 0 or 1; and

T is H, C(O)CH₃, or Z.

In yet another embodiment, compounds of the Formula IIa are disclosed:

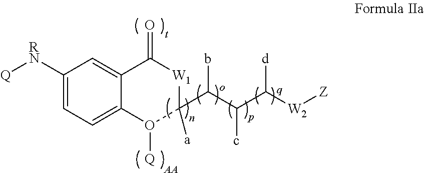

or a pharmaceutically acceptable salt, hydrate, solvate, prodrug, enantiomer, or stereoisomer thereof; wherein

each W₁ and W₂ are independently a bond, O, or —N(R)—, or when W₁ and W₂ are both NH, then both W₁ and W₂ can be taken together to form a piperidine moiety;

[symbol] represents an optional bond that when present requires that AA is 0;

each a and c are independently H, CH₃, —OCH₃, —OCH₂CH₃, or C(O)OH;

each b is H, CH₃, C(O)OH, or O—Z;

each d is H or C(O)OH;

each n, o, p, and q is independently 0 or 1;

each Z is H or

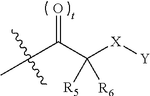

with the proviso that there is at least one

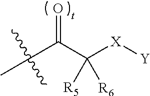

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

each Q is H, C(O)CH₃, Z,

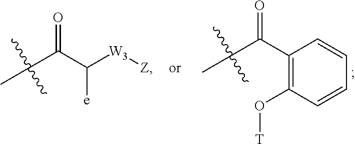

each e is H or any one of the side chains of naturally occurring amino acids;

W₃ is a bond, —O—, or —N(R)—;

R is H or C₁-C₃ alkyl;

AA is 0 or 1; and

T is H, C(O)CH₃, or Z.

In another embodiment, compounds of Formula III are disclosed herein:

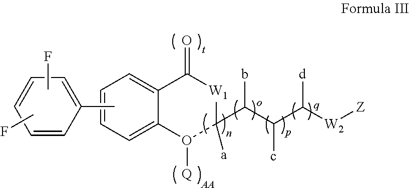

or a pharmaceutically acceptable salt, hydrate, solvate, prodrug, enantiomer, or stereoisomer thereof; wherein

W₁ and W₂ are each independently a bond, O, or —N(R)—;

[symbol] represents an optional bond that when present requires that AA is 0;

a and c are each independently H, CH₃, —OCH₃, —OCH₂CH₃, or C(O)OH;

b is H, CH₃, C(O)OH, or O—Z;

d is H or C(O)OH; each n, o, p, and q is independently 0 or 1;

each Z is H or

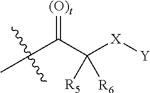

with the proviso that there is at least one

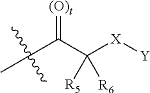

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

Q is H, C(O)CH₃, Z, or

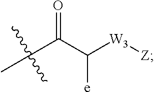

e is H, —C(O)OH, or any one of the side chains of the naturally occurring amino acids;

W₃ is a bond, —O—, —N(R)—;

AA is 0 or 1; and

R is H or C₁-C₃ alkyl.

In another embodiment, compounds of the Formula IIIa are disclosed:

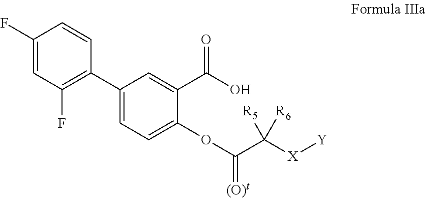

or a pharmaceutically acceptable salt, hydrate, solvate, prodrug, enantiomer, or stereoisomer thereof, wherein

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds; and

t is 0 or 1.

In another embodiment, compounds of the Formula IIIb are disclosed:

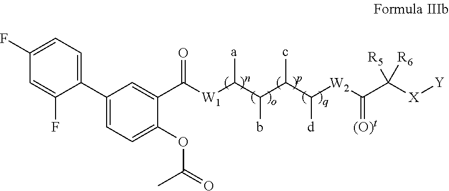

and pharmaceutically acceptable salts, hydrates, solvates, enantiomers, and stereoisomers thereof, wherein

W₁ and W₂ are each independently a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

Z is H or

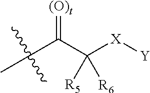

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula IIIc are disclosed:

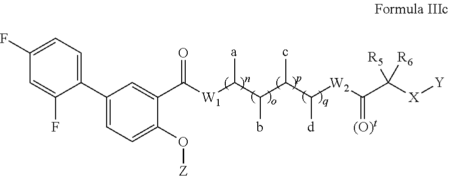

or a pharmaceutically acceptable salt, hydrate, solvate, prodrug, enantiomer, or stereoisomer thereof; wherein

W₁ and W₂ are each independently a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or C(O)OH;

each of n, o, p, and q is independently 0 or 1;

each Z is independently H, or

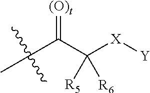

with the proviso that there is at least one

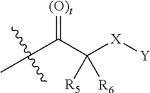

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula IIId are disclosed:

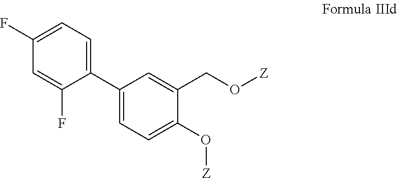

or a pharmaceutically acceptable salt, hydrate, solvate, prodrug, enantiomer, or stereoisomer thereof;

wherein each Z is independently H, or

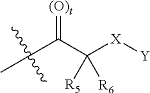

with the proviso that there is at least one

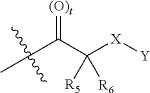

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula IIIe are disclosed:

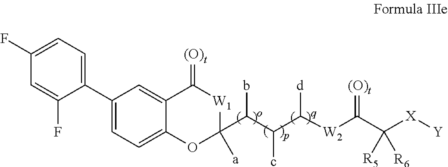

or a pharmaceutically acceptable salt, hydrate, solvate, prodrug, enantiomer, or stereoisomer thereof; wherein

W₁ is O or —N(R)—;

W₂ is a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or C(O)OH;

each o, p, and q is independently 0 or 1;

Z is H or

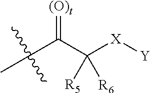

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula IIIf are disclosed:

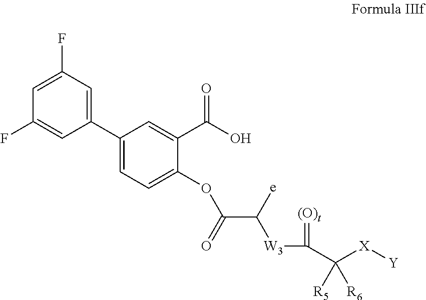

or a pharmaceutically acceptable salt, hydrate, solvate, prodrug, enantiomer, or stereoisomer thereof; wherein

e is H, —C(O)OH or any one of the side chains of the naturally occurring amino acids;

W₃ is a bond, —O—, —N(R)—;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

t is 0 or 1;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds; and

R is H or C₁-C₃ alkyl.

In another embodiment, compounds of the Formula IIIg are disclosed:

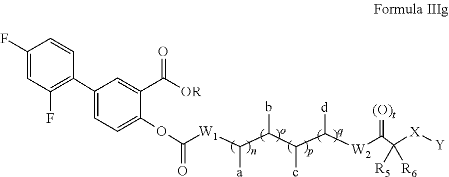

or a pharmaceutically acceptable salt, hydrate, solvate, prodrug, enantiomer, or stereoisomer thereof; wherein

R is H or C₁-C₃ alkyl;

W₁ and W₂ are each independently a bond, O, or —N(R)—;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

Z is H or

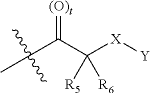

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In still another embodiment, compounds of the Formula IV are disclosed:

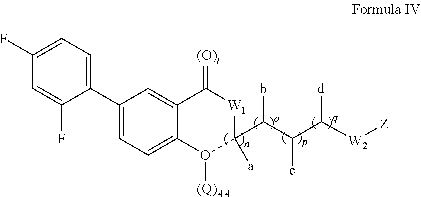

or a pharmaceutically acceptable salt, hydrate, solvate, prodrug, enantiomer, or stereoisomer thereof; wherein

W₁ and W₂ are each independently a bond, O, or —N(R)—;

[symbol] represents an optional bond that when present requires that AA is 0;

a and c are each independently H, CH₃, —OCH₃, —OCH₂CH₃, or C(O)OH;

b is H, CH₃, C(O)OH, or O—Z;

d is H or C(O)OH; each n, o, p, and q is independently 0 or 1;

each Z is independently H, or

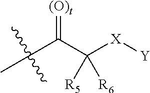

with the proviso that there is at least one

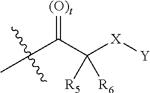

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

Q is H, C(O)CH₃, Z, or

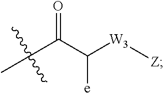

W₃ is a bond, —O—, —N(R)—;

R is H or C₁-C₃ alkyl;

AA is 0 or 1; and

e is H, —C(O)OH or any one of the side chains of the naturally occurring amino acids.

In yet another embodiment, compounds of the Formula V are disclosed:

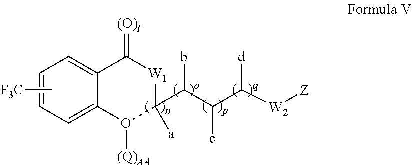

or a pharmaceutically acceptable salt, hydrate, solvate, prodrug, enantiomer, or stereoisomer thereof; wherein

W₁ and W₂ are each independently a bond, O, or —N(R)—;

[symbol] represents an optional bond that when present requires that AA is 0;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

each Z is H or

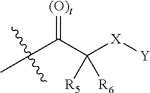

with the proviso that there is at least one

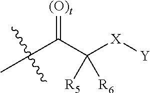

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

Q is H, C(O)CH₃, Z, or

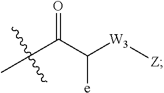

e is H, —C(O)OH, or any one of the side chains of the naturally occurring amino acids;

W₃ is a bond, —O—, —N(R)—;

AA is 0 or 1; and

R is H or C₁-C₃ alkyl.

In another embodiment, compounds of the Formula Va are disclosed:

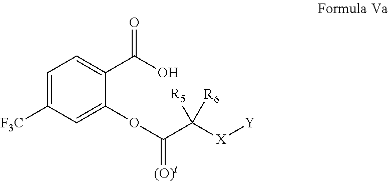

or a pharmaceutically acceptable salt, hydrate, solvate, prodrug, enantiomer, or stereoisomer thereof, wherein

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Vb are disclosed:

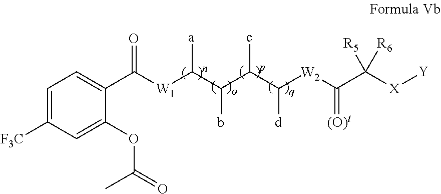

and pharmaceutically acceptable salts, hydrates, solvates, enantiomers, and stereoisomers thereof, wherein

W₁ and W₂ are each independently a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

Z is H or

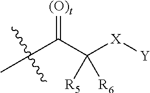

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Vc are disclosed:

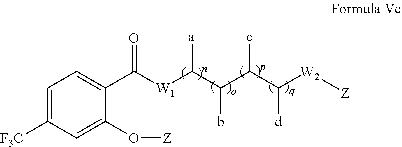

or a pharmaceutically acceptable salt, hydrate, solvate, prodrug, enantiomer, or stereoisomer thereof; wherein

W₁ and W₂ are each independently a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or C(O)OH;

each of n, o, p, and q is independently 0 or 1;

each Z is independently H, or

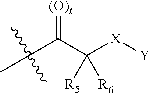

with the proviso that there is at least one

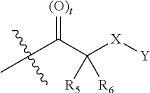

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Vd are disclosed:

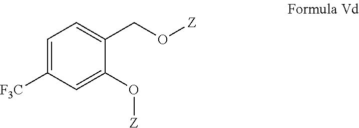

or a pharmaceutically acceptable salt, hydrate, solvate, prodrug, enantiomer, or stereoisomer thereof; wherein

each Z is independently H, —C(O)CH₃ or

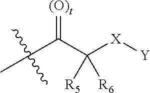

with the proviso that there is at least one

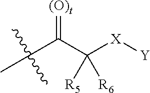

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Ve are disclosed:

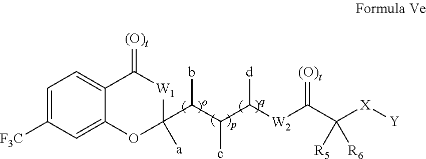

or a pharmaceutically acceptable salt, hydrate, solvate, prodrug, enantiomer, or stereoisomer thereof; wherein

W₁ is O or —N(R)—;

W₂ is a bond, O, or —N(R)—, wherein R is H or C₁-C₃ alkyl;

a and c are each independently H, CH₃, —OCH₃, —OCH₂CH₃, or C(O)OH;

b is H, CH₃, C(O)OH, or O—Z;

d is H or O(O)OH;

each o, p, and q is independently 0 or 1;

Z is H or

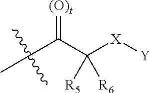

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula Vf are disclosed:

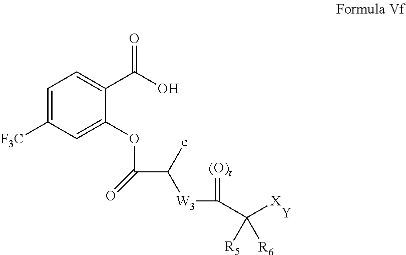

or a pharmaceutically acceptable salt, hydrate, solvate, prodrug, enantiomer, or stereoisomer thereof; wherein

e is H, —C(O)OH or any one of the side chains of the naturally occurring amino acids;

W₃ is a bond, —O—, —N(R)—;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

t is 0 or 1;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds; and

R is H or C₁-C₃ alkyl.

In another aspect, compounds of the Formula Vg are disclosed:

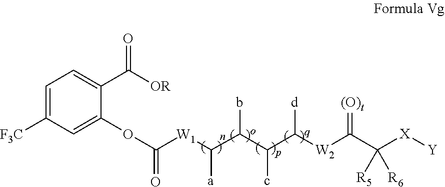

or a pharmaceutically acceptable salt, hydrate, solvate, prodrug, enantiomer, or stereoisomer thereof; wherein

R is H or C₁-C₃ alkyl;

W₁ and W₂ are each independently a bond, O, or —N(R)—;

a and c are each independently H, CH₃, —OCH₃, —OCH₂CH₃, or C(O)OH,

b is H, CH₃, C(O)OH, or O—Z;

d is H or C(O)OH;

each n, o, p, and q is independently 0 or 1;

Z is H or

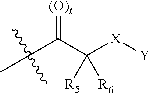

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds.

In another embodiment, compounds of the Formula VI are disclosed:

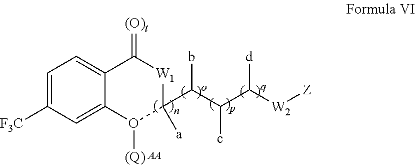

or a pharmaceutically acceptable salt, hydrate, solvate, prodrug, enantiomer, or stereoisomer thereof; wherein

W₁ and W₂ are each independently a bond, O, or —N(R)—;

[symbol] represents an optional bond that when present requires that AA is 0;

a and c are each independently H, CH₃, —OCH₃, —OCH₂CH₃, or C(O)OH;

b is H, CH₃, C(O)OH, or O—Z;

d is H or C(O)OH;

each n, o, p, and q is independently 0 or 1;

each Z is H or

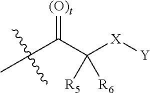

with the proviso that there is at least one

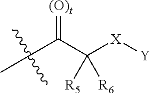

in the compound;

each t is independently 0 or 1;

each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;

X is chosen from —CH₂—, O, S, SO, and SO₂;

Y is chosen from a C₁₀-C₂₄ alkyl, a C₁₀-C₂₄ alkenyl having 1-6 double bonds, and a C₁₀-C₂₂ alkynyl having 1-6 triple bonds;

Q is H, C(O)CH₃, Z, or

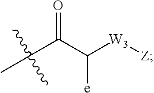

W₃ is a bond, —O—, —N(R)—;

R is H or C₁-C₃ alkyl;

AA is 0 or 1; and

e is H, —C(O)OH or any one of the side chains of the naturally occurring amino acids.

In any of the above formulas, any one or more of H may be substituted with a deuterium. It is also understood in any of the above Formulae that a methyl substituent can be substituted with a C₁-C₆ alkyl. Moreover, in at least one embodiment R₅ and R₆ are not both hydrogen.

Also disclosed are pharmaceutical formulations comprising at least one compound according to the formulas disclosed herein.

Also disclosed herein are methods of simultaneously up regulating anti-inflammation pathways and down regulating pro-inflammation pathways in a cell by administering to the cell at least one compound according to the present disclosure.

Also disclosed herein are methods of simultaneously up regulating anti-inflammation pathways and down regulating pro-inflammation pathways in a patient in need thereof, by administering to the patient an effective amount of at least one compound according to the present disclosure.

Also disclosed herein are methods of treating a disease susceptible to treatment with at least one compound according to the present disclosure in a patient in need thereof by administering to the patient an effective amount of at least one compound according to the present disclosure.

Also disclosed herein are methods of treating diseases associated with inflammation by administering to a patient in need thereof an effective amount of at least one compound according to the present disclosure.

Also disclosed herein are methods of preventing and/or treating inflammatory bowel disease (IBD), diabetes, mixed dyslipidemia, metabolic syndrome, peripheral insulin resistance, and/or atherosclerosis by administering to a patient in need thereof an effective amount of at least one compound according to the present disclosure.

Also disclosed herein are methods of lowering cholesterol, such as non-HDL cholesterol, e.g., LDL cholesterol and VLDL cholesterol, by administering to a patient in need thereof an effective amount of at least one compound according to the present disclosure.

Also disclosed herein are methods of raising HDL cholesterol by administering to a patient in need thereof an effective amount of at least one compound according to the present disclosure.

Also disclosed herein are methods of decreasing triglycerides, such as a method of preventing or treating hypertriglyceridemia by administering to a patient in need thereof an effective amount of at least one compound according to the present disclosure.

The present disclosure also includes pharmaceutical compositions that comprise an effective amount of at least one compound according to the present disclosure and a pharmaceutically acceptable carrier. The compositions are useful for treating or preventing an inflammatory disease, as well as the other diseases and conditions disclosed herein. The present disclosure includes a compound of the present disclosure when provided as a pharmaceutically acceptable prodrug, a hydrate, a salt, such as a pharmaceutically acceptable salt, enantiomer, stereoisomer, or mixtures thereof.

The details of the present disclosure are set forth in the accompanying description below. Although any methods and materials similar or equivalent to those disclosed herein can be used in the practice or testing of the present disclosure, illustrative methods and materials are now disclosed. Other features, objects, and advantages of the present disclosure will be apparent from the description and from the claims. In the specification and the appended claims, the singular forms also include the plural unless the context clearly dictates otherwise.

### DETAILED DESCRIPTION

### Definitions

The following definitions are used in connection with the fatty acid conjugates of salicylate derivatives:

The term “compound of the present disclosure” refers to a fatty acid conjugate of salicylate derivative disclosed herein, wherein salicylate derivative includes, without limitation, salicylic acid and substituted salicylates such as aminosalicylic acid, a diflunisal derivative or a triflusal derivative. The term “compounds of the present disclosure” refers to more than one compound of the present disclosure and may be fatty acid conjugates of salicylate derivatives or some combination thereof. The compounds of the present disclosure include any and all possible isomers, stereoisomers, enantiomers, diastereomers, tautomers, pharmaceutically acceptable salts, hydrates, solvates, and prodrugs thereof.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

A “subject” or “patient” is a mammal, e.g., a human, mouse, rat, guinea pig, dog, cat, horse, cow, pig, or non-human primate, such as a monkey, chimpanzee, baboon or rhesus.

The present disclosure also includes pharmaceutical compositions comprising an effective amount of at least one compound of the present disclosure and a pharmaceutically acceptable carrier. The present disclosure includes a compound of the present disclosure when provided as a pharmaceutically acceptable prodrug, hydrate, salt, such as a pharmaceutically acceptable salt, enantiomers, stereoisomers, or mixtures thereof.

Exemplary “pharmaceutically acceptable salts” include, e.g., water-soluble and water-insoluble salts, such as the acetate, amsonate (4,4-diaminostilbene-2,2-disulfonate), benzenesulfonate, benzonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, subsalicylate, suramate, tannate, tartrate, teoclate, tosylate, triethiodide, and valerate salts.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a compound of the present disclosure.

The present disclosure provides fatty acid conjugates of salicylate derivatives according to Formula I, Formula Ia, Formula Ib, Formula Ic, Formula Id, Formula Ie, Formula If, Formula Ig, Formula II, Formula IIa, Formula III, Formula IIIa, Formula IIIb, Formula IIIc, Formula IIId, Formula IIIe, Formula IIIf, Formula IIIg, Formula IV, Formula V, Formula Va, Formula Vb, Formula Vc, Formula Vd, Formula Ve, Formula Vf, Formula Vg, and Formula VI, as set forth herein.

Exemplary embodiments are provided by the following categories A-I, wherein R₅, R₆, X, and Y are hereinabove defined.

Category A:

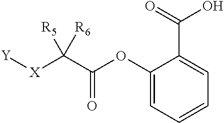

Examples:

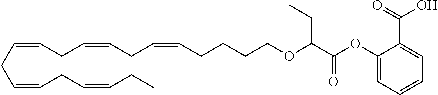 [SMILES: CC/C=C\C/C=C\C/C=C\C/C=C\C/C=C\CCCCOC(CC)C(=O)Oc1ccccc1C(=O)O]

2-((2-((5Z,8Z,11Z,14Z,17Z)-icosa-5,8,11,14,17-pentaen-1-yloxy)butanoyl)oxy)benzoic acid;

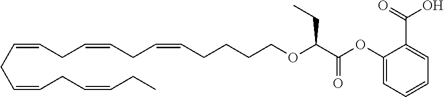 [SMILES: CC/C=C\C/C=C\C/C=C\C/C=C\C/C=C\CCCCO[C@@H](CC)C(=O)Oc1ccccc1C(=O)O]

2-(((S)-2-((5Z,8Z,11Z,14Z,17Z)-icosa-5,8,11,14,17-pentaen-1-yloxy)butanoyl)oxy)benzoic acid;

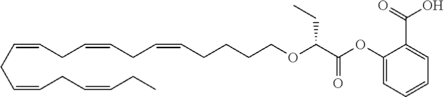 [SMILES: CC/C=C\C/C=C\C/C=C\C/C=C\C/C=C\CCCCO[C@H](CC)C(=O)Oc1ccccc1C(=O)O]

2-(((R)-2-((5Z,8Z,11Z,14Z,17Z)-icosa-5,8,11,14,17-pentaen-1-yloxy)butanoyl)oxy)benzoic acid;

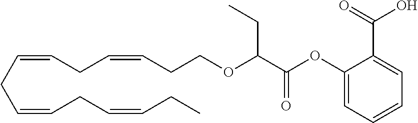 [SMILES: CC/C=C\C/C=C\C/C=C\C/C=C\CCOC(CC)C(=O)Oc1ccccc1C(=O)O]

2-((2-((3Z,6Z,9Z,12Z)-pentadeca-3,6,9,12-tetraen-1-yloxy)butanoyl)oxy)benzoic acid;

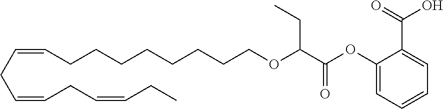 [SMILES: CC/C=C\C/C=C\C/C=C\CCCCCCCCOC(CC)C(=O)Oc1ccccc1C(=O)O]

2-((2-((9Z,12Z,15Z)-octadeca-9,12,15-trien-1-yloxy)butanoyl)oxy)benzoic acid;

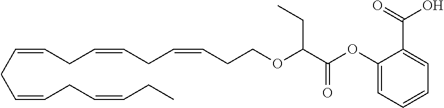 [SMILES: CC/C=C\C/C=C\C/C=C\C/C=C\C/C=C\CCOC(CC)C(=O)Oc1ccccc1C(=O)O]

2-((2-((3Z,6Z,9Z,12Z,15Z)-octadeca-3,6,9,12,15-pentaen-1-yloxy)butanoyl)oxy)benzoic acid;

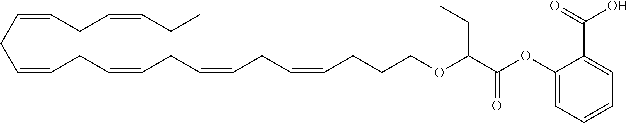 [SMILES: CC/C=C\C/C=C\C/C=C\C/C=C\C/C=C\C/C=C\CCCOC(CC)C(=O)Oc1ccccc1C(=O)O]

2-((2-((4Z,7Z,10Z,13Z,16Z,19Z)-docosa-4,7,10,13,16,19-hexaen-1-yloxy)butanoyl)oxy)benzoic acid;

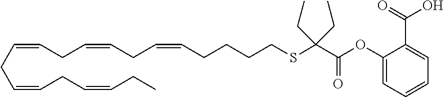 [SMILES: CC/C=C\C/C=C\C/C=C\C/C=C\C/C=C\CCCCSC(CC)(CC)C(=O)Oc1ccccc1C(=O)O]

2-((2-ethyl-2-((5Z,8Z,11Z,14Z,17Z)-icosa-5,8,11,14,17-pentaen-1-ylthio)butanoyl)oxy)benzoic acid;

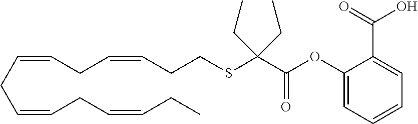 [SMILES: CC/C=C\C/C=C\C/C=C\C/C=C\CCSC(CC)(CC)C(=O)Oc1ccccc1C(=O)O]

2-((2-ethyl-2-((3Z,6Z,9Z,12Z)-pentadeca-3,6,9,12-tetraen-1-ylthio)butanoyl)oxy)benzoic acid;

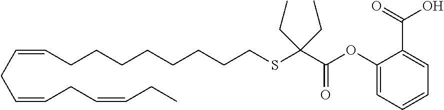 [SMILES: CC/C=C\C/C=C\C/C=C\CCCCCCCCSC(CC)(CC)C(=O)Oc1ccccc1C(=O)O]

2-((2-ethyl-2-((9Z,12Z,15Z)-octadeca-9,12,15-trien-1-ylthio)butanoyl)oxy)benzoic acid;

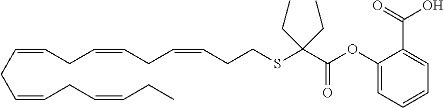 [SMILES: CC/C=C\C/C=C\C/C=C\C/C=C\C/C=C\CCSC(CC)(CC)C(=O)Oc1ccccc1C(=O)O]

2-((2-ethyl-2-((3Z,6Z,9Z,12Z,15Z)-octadeca-3,6,9,12,15-pentaen-1-ylthio)butanoyl)oxy)benzoic acid;

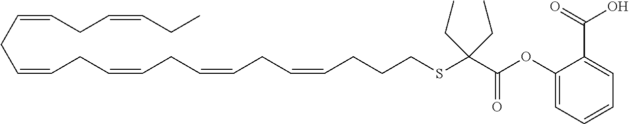 [SMILES: CC/C=C\C/C=C\C/C=C\C/C=C\C/C=C\C/C=C\CCCSC(CC)(CC)C(=O)Oc1ccccc1C(=O)O]

2-((2-((4Z,7Z,10Z,13Z,16Z,19Z)-docosa-4,7,10,13,16,19-hexaen-1-ylthio)-2-ethylbutanoyl)oxy)benzoic acid;

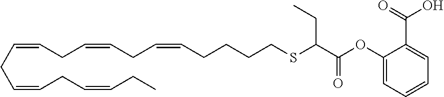 [SMILES: CC/C=C\C/C=C\C/C=C\C/C=C\C/C=C\CCCCSC(CC)C(=O)Oc1ccccc1C(=O)O]

2-((2-((5Z,8Z,11Z,14Z,17Z)-icosa-5,8,11,14,17-pentaen-1-ylthio)butanoyl)oxy)benzoic acid;

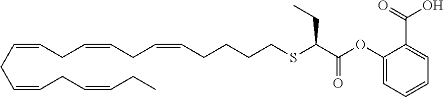 [SMILES: CC/C=C\C/C=C\C/C=C\C/C=C\C/C=C\CCCCS[C@@H](CC)C(=O)Oc1ccccc1C(=O)O]

2-(((S)-2-((5Z,8Z,11Z,14Z,17Z)-icosa-5,8,11,14,17-pentaen-1-ylthio)butanoyl)oxy)benzoic acid;

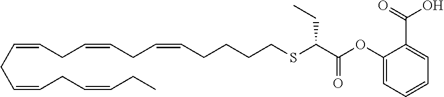 [SMILES: CC/C=C\C/C=C\C/C=C\C/C=C\C/C=C\CCCCS[C@H](CC)C(=O)Oc1ccccc1C(=O)O]

2-(((R)-2-((5Z,8Z,11Z,14Z,17Z)-icosa-5,8,11,14,17-pentaen-1-ylthio)butanoyl)oxy)benzoic acid;

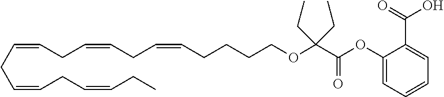 [SMILES: CC/C=C\C/C=C\C/C=C\C/C=C\C/C=C\CCCCOC(CC)(CC)C(=O)Oc1ccccc1C(=O)O]

2-((2-ethyl-2-((5Z,8Z,11Z,14Z,17Z)-icosa-5,8,11,14,17-pentaen-1-yloxy)butanoyl)oxy)benzoic acid;

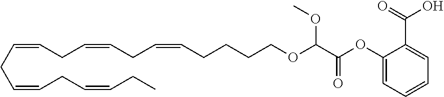 [SMILES: CC/C=C\C/C=C\C/C=C\C/C=C\C/C=C\CCCCOC(OC)C(=O)Oc1ccccc1C(=O)O]

2-(2-((5Z,8Z,11Z,14Z,17Z)-icosa-5,8,11,14,17-pentaen-1-yloxy)-2-methoxyacetoxy)benzoic acid;

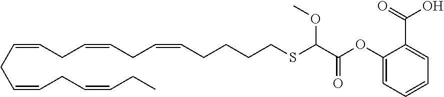 [SMILES: CC/C=C\C/C=C\C/C=C\C/C=C\C/C=C\CCCCSC(OC)C(=O)Oc1ccccc1C(=O)O]

2-(2-((5Z,8Z,11Z,14Z,17Z)-icosa-5,8,11,14,17-pentaen-1-ylthio)-2-methoxyacetoxy)benzoic acid;

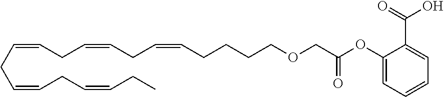 [SMILES: CC/C=C\C/C=C\C/C=C\C/C=C\C/C=C\CCCCOCC(=O)Oc1ccccc1C(=O)O]

2-(2-((5Z,8Z,11Z,14Z,17Z)-icosa-5,8,11,14,17-pentaen-1-yloxy)acetoxy)benzoic acid; and

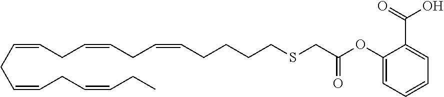 [SMILES: CC/C=C\C/C=C\C/C=C\C/C=C\C/C=C\CCCCSCC(=O)Oc1ccccc1C(=O)O]

2-(2-((5Z,8Z,11Z,14Z,17Z)-icosa-5,8,11,14,17-pentaen-1-ylthio)acetoxy)benzoic acid.

Category B:

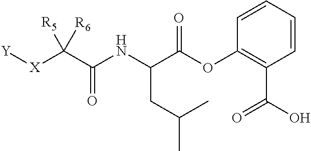

Examples:

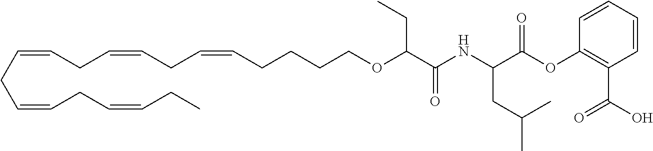 [SMILES: CC/C=C\C/C=C\C/C=C\C/C=C\C/C=C\CCCCOC(CC)C(=O)NC(CC(C)C)C(=O)Oc1ccccc1C(=O)O]

2-((2-(2-((5Z,8Z,11Z,14Z,17Z)-icosa-5,8,11,14,17-pentaen-1-yloxy)butanamido)-4-methylpentanoyl)oxy)benzoic acid;

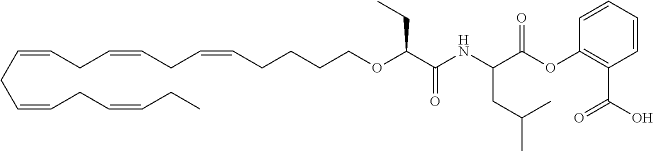 [SMILES: CC/C=C\C/C=C\C/C=C\C/C=C\C/C=C\CCCCO[C@@H](CC)C(=O)NC(CC(C)C)C(=O)Oc1ccccc1C(=O)O]

2-((2-((S)-2-((5Z,8Z,11Z,14Z,17Z)-icosa-5,8,11,14,17-pentaen-1-yloxy)butanamido)-4-methylpentanoyl)oxy)benzoic acid;

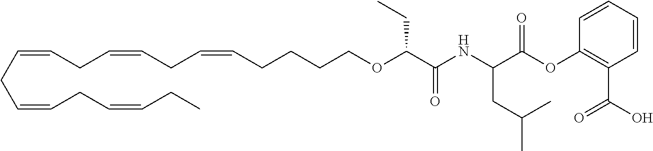 [SMILES: CC/C=C\C/C=C\C/C=C\C/C=C\C/C=C\CCCCO[C@H](CC)C(=O)NC(CC(C)C)C(=O)Oc1ccccc1C(=O)O]

2-((2-((R)-2-((5Z,8Z,11Z,14Z,17Z)-icosa-5,8,11,14,17-pentaen-1-yloxy)butanamido)-4-methylpentanoyl)oxy)benzoic acid;

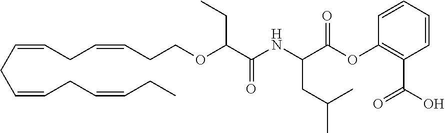 [SMILES: CC/C=C\C/C=C\C/C=C\C/C=C\CCOC(CC)C(=O)NC(CC(C)C)C(=O)Oc1ccccc1C(=O)O]

2-((4-methyl-2-(2-((3Z,6Z,9Z,12Z)-pentadeca-3,6,9,12-tetraen-1-yloxy)butanamido)pentanoyl)oxy)benzoic acid;

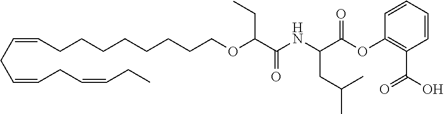 [SMILES: CC/C=C\C/C=C\C/C=C\CCCCCCCCOC(CC)C(=O)NC(CC(C)C)C(=O)Oc1ccccc1C(=O)O]

2-((4-methyl-2-(2-((9Z,12Z,15Z)-octadeca-9,12,15-trien-1-yloxy)butanamido)pentanoyl)oxy)benzoic acid;

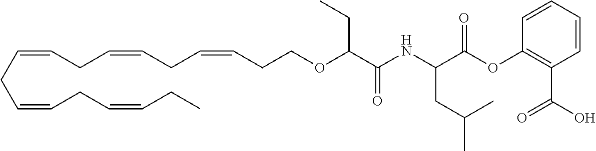 [SMILES: CC/C=C\C/C=C\C/C=C\C/C=C\C/C=C\CCOC(CC)C(=O)NC(CC(C)C)C(=O)Oc1ccccc1C(=O)O]

2-((4-methyl-2-(2-((3Z,6Z,9Z,12Z,15Z)-octadeca-3,6,9,12,15-pentaen-1-yloxy)butanamido)pentanoyl)oxy)benzoic acid;

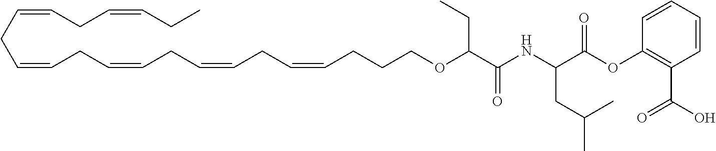 [SMILES: CC/C=C\C/C=C\C/C=C\C/C=C\C/C=C\C/C=C\CCCOC(CC)C(=O)NC(CC(C)C)C(=O)Oc1ccccc1C(=O)O]

2-((2-(2-((4Z,7Z,10Z,13Z,16Z,19Z)-docosa-4,7,10,13,16,19-hexaen-1-yloxy)butanamido)-4-methylpentanoyl)oxy)benzoic acid;

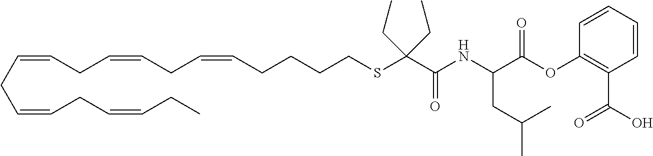 [SMILES: CC/C=C\C/C=C\C/C=C\C/C=C\C/C=C\CCCCSC(CC)(CC)C(=O)NC(CC(C)C)C(=O)Oc1ccccc1C(=O)O]

2-((2-(2-ethyl-2-((5Z,8Z,11Z,14Z,17Z)-icosa-5,8,11,14,17-pentaen-1-ylthio)butanamido)-4-methylpentanoyl)oxy)benzoic acid;

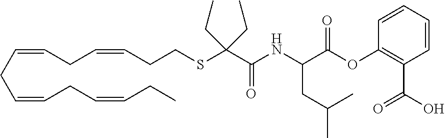 [SMILES: CC/C=C\C/C=C\C/C=C\C/C=C\CCSC(CC)(CC)C(=O)NC(CC(C)C)C(=O)Oc1ccccc1C(=O)O]

2-((2-(2-ethyl-2-((3Z,6Z,9Z,12Z)-pentadeca-3,6,9,12-tetraen-1-ylthio)butanamido)-4-methylpentanoyl)oxy)benzoic acid;

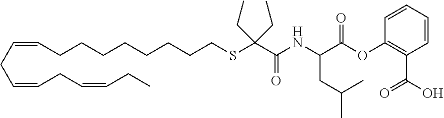 [SMILES: CC/C=C\C/C=C\C/C=C\CCCCCCCCSC(CC)(CC)C(=O)NC(CC(C)C)C(=O)Oc1ccccc1C(=O)O]

2-((2-(2-ethyl-2-((9Z,12Z,15Z)-octadeca-9,12,15-trien-1-ylthio)butanamido)-4-methylpentanoyl)oxy)benzoic acid;

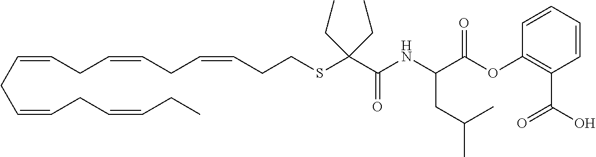 [SMILES: CC/C=C\C/C=C\C/C=C\C/C=C\C/C=C\CCSC(CC)(CC)C(=O)NC(CC(C)C)C(=O)Oc1ccccc1C(=O)O]

2-((2-(2-ethyl-2-((3Z,6Z,9Z,12Z,15Z)-octadeca-3,6,9,12,15-pentaen-1-ylthio)butanamido)-4-methylpentanoyl)oxy)benzoic acid;

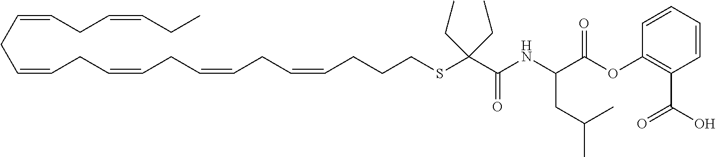 [SMILES: CC/C=C\C/C=C\C/C=C\C/C=C\C/C=C\C/C=C\CCCSC(CC)(CC)C(=O)NC(CC(C)C)C(=O)Oc1ccccc1C(=O)O]

2-((2-(2-((4Z,7Z,10Z,13Z,16Z,19Z)-docosa-4,7,10,13,16,19-hexaen-1-ylthio)-2-ethylbutanamido)-4-methylpentanoyl)oxy)benzoic acid;

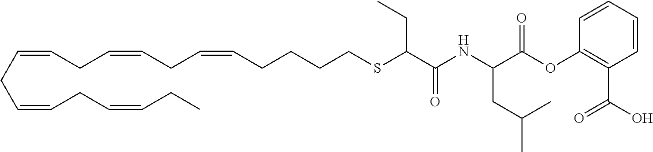 [SMILES: CC/C=C\C/C=C\C/C=C\C/C=C\C/C=C\CCCCSC(CC)C(=O)NC(CC(C)C)C(=O)Oc1ccccc1C(=O)O]

2-((2-(2-((5Z,8Z,11Z,14Z,17Z)-icosa-5,8,11,14,17-pentaen-1-ylthio)butanamido)-4-methylpentanoyl)oxy)benzoic acid;

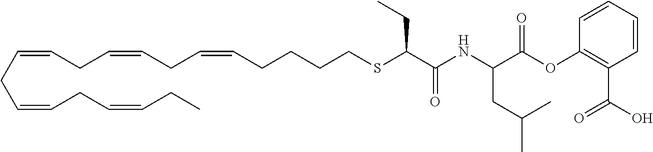 [SMILES: CC/C=C\C/C=C\C/C=C\C/C=C\C/C=C\CCCCS[C@@H](CC)C(=O)NC(CC(C)C)C(=O)Oc1ccccc1C(=O)O]

2-((2-((S)-2-((5Z,8Z,11Z,14Z,17Z)-icosa-5,8,11,14,17-pentaen-1-ylthio)butanamido)-4-methylpentanoyl)oxy)benzoic acid;

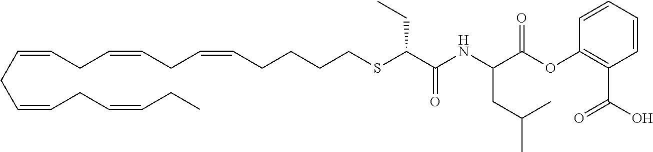 [SMILES: CC/C=C\C/C=C\C/C=C\C/C=C\C/C=C\CCCCS[C@H](CC)C(=O)NC(CC(C)C)C(=O)Oc1ccccc1C(=O)O]

2-((2-((R)-2-((5Z,8Z,11Z,14Z,17Z)-icosa-5,8,11,14,17-pentaen-1-ylthio)butanamido)-4-methylpentanoyl)oxy)benzoic acid;

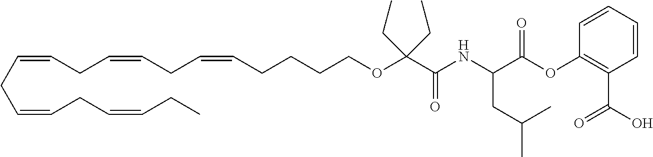 [SMILES: CC/C=C\C/C=C\C/C=C\C/C=C\C/C=C\CCCCOC(CC)(CC)C(=O)NC(CC(C)C)C(=O)Oc1ccccc1C(=O)O]

2-((2-(2-ethyl-2-((5Z,8Z,11Z,14Z,17Z)-icosa-5,8,11,14,17-pentaen-1-yloxy)butanamido)-4-methylpentanoyl)oxy)benzoic acid;

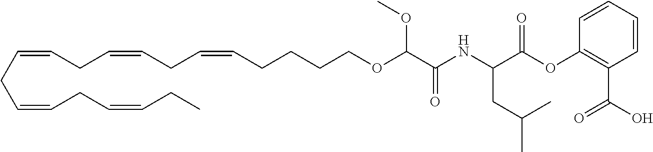 [SMILES: CC/C=C\C/C=C\C/C=C\C/C=C\C/C=C\CCCCOC(OC)C(=O)NC(CC(C)C)C(=O)Oc1ccccc1C(=O)O]

2-((2-(2-((5Z,8Z,11Z,14Z,17Z)-icosa-5,8,11,14,17-pentaen-1-yloxy)-2-methoxyacetamido)-4-methylpentanoyl)oxy)benzoic acid;

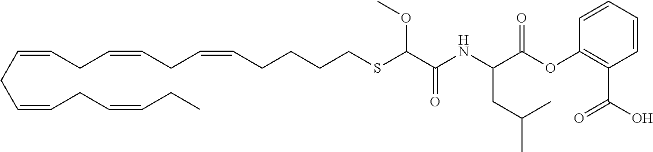 [SMILES: CC/C=C\C/C=C\C/C=C\C/C=C\C/C=C\CCCCSC(OC)C(=O)NC(CC(C)C)C(=O)Oc1ccccc1C(=O)O]

2-((2-(2-((5Z,8Z,11Z,14Z,17Z)-icosa-5,8,11,14,17-pentaen-1-ylthio)-2-methoxyacetamido)-4-methylpentanoyl)oxy)benzoic acid;

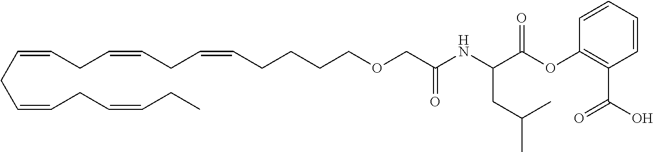 [SMILES: CC/C=C\C/C=C\C/C=C\C/C=C\C/C=C\CCCCOCC(=O)NC(CC(C)C)C(=O)Oc1ccccc1C(=O)O]

2-((2-(2-((5Z,8Z,11Z,14Z,17Z)-icosa-5,8,11,14,17-pentaen-1-yloxy)acetamido)-4-methylpentanoyl)oxy)benzoic acid; and

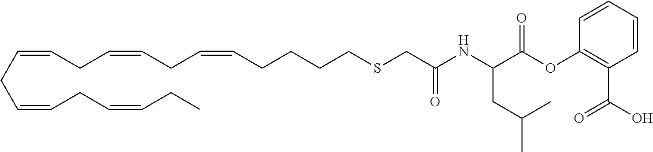 [SMILES: CC/C=C\C/C=C\C/C=C\C/C=C\C/C=C\CCCCSCC(=O)NC(CC(C)C)C(=O)Oc1ccccc1C(=O)O]

2-((2-(2-((5Z,8Z,11Z,14Z,17Z)-icosa-5,8,11,14,17-pentaen-1-ylthio)acetamido)-4-methylpentanoyl)oxy)benzoic acid.

Category C:

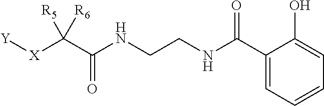

Examples:

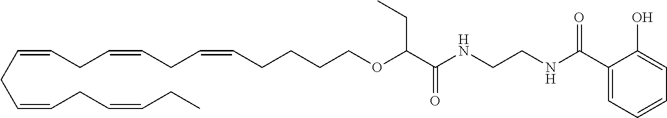 [SMILES: CC/C=C\C/C=C\C/C=C\C/C=C\C/C=C\CCCCOC(CC)C(=O)NCCNC(=O)c1ccccc1O]

2-hydroxy-N-(2-(2-((5Z,8Z,11Z,14Z,17Z)-icosa-5,8,11,14,17-pentaen-1-yloxy)butanamido)ethyl)benzamide;

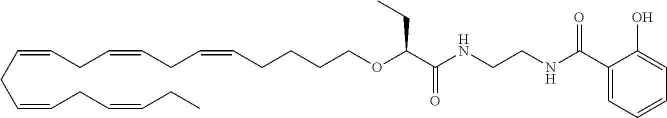 [SMILES: CC/C=C\C/C=C\C/C=C\C/C=C\C/C=C\CCCCO[C@@H](CC)C(=O)NCCNC(=O)c1ccccc1O]

2-hydroxy-N-(2-((S)-2-((5Z,8Z,11Z,14Z,17Z)-icosa-5,8,11,14,17-pentaen-1-yloxy)butanamido)ethyl)benzamide;

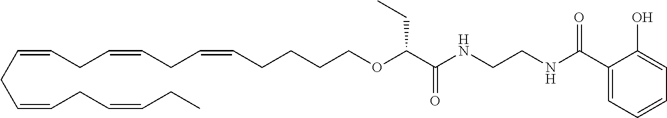 [SMILES: CC/C=C\C/C=C\C/C=C\C/C=C\C/C=C\CCCCO[C@H](CC)C(=O)NCCNC(=O)c1ccccc1O]

2-hydroxy-N-(2-((R)-2-((5Z,8Z,11Z,14Z,17Z)-icosa-5,8,11,14,17-pentaen-1-yloxy)butanamido)ethyl)benzamide;

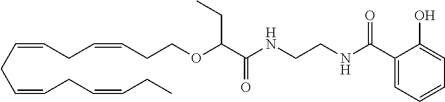 [SMILES: CC/C=C\C/C=C\C/C=C\C/C=C\CCOC(CC)C(=O)NCCNC(=O)c1ccccc1O]

2-hydroxy-N-(2-(2-((3Z,6Z,9Z,12Z)-pentadeca-3,6,9,12-tetraen-1-yloxy)butanamido)ethyl)benzamide;

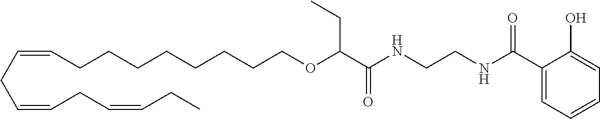 [SMILES: CC/C=C\C/C=C\C/C=C\CCCCCCCCOC(CC)C(=O)NCCNC(=O)c1ccccc1O]

2-hydroxy-N-(2-(2-((9Z,12Z,15Z)-octadeca-9,12,15-trien-1-yloxy)butanamido)ethyl)benzamide;

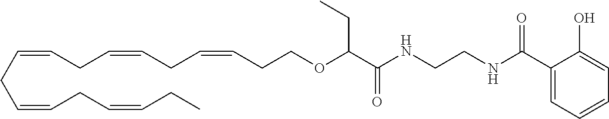 [SMILES: CC/C=C\C/C=C\C/C=C\C/C=C\C/C=C\CCOC(CC)C(=O)NCCNC(=O)c1ccccc1O]

2-hydroxy-N-(2-(2-((3Z,6Z,9Z,12Z,15Z)-octadeca-3,6,9,12,15-pentaen-1-yloxy)butanamido)ethyl)benzamide;

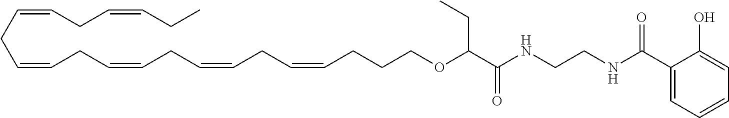 [SMILES: CC/C=C\C/C=C\C/C=C\C/C=C\C/C=C\C/C=C\CCCOC(CC)C(=O)NCCNC(=O)c1ccccc1O]

N-(2-(2-((4Z,7Z,10Z,13Z,16Z,19Z)-docosa-4,7,10,13,16,19-hexaen-1-yloxy)butanamido)ethyl)-2-hydroxybenzamide;

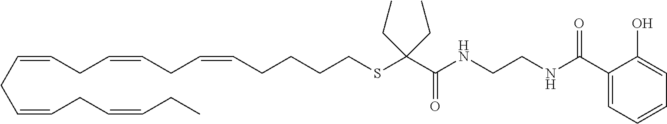 [SMILES: CC/C=C\C/C=C\C/C=C\C/C=C\C/C=C\CCCCSC(CC)(CC)C(=O)NCCNC(=O)c1ccccc1O]

N-(2-(2-ethyl-2-((5Z,8Z,11Z,14Z,17Z)-icosa-5,8,11,14,17-pentaen-1-ylthio)butanamido)ethyl)-2-hydroxybenzamide;

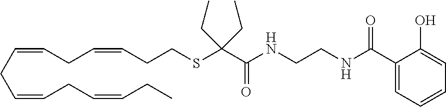 [SMILES: CC/C=C\C/C=C\C/C=C\C/C=C\CCSC(CC)(CC)C(=O)NCCNC(=O)c1ccccc1O]

N-(2-(2-ethyl-2-((3Z,6Z,9Z,12Z)-pentadeca-3,6,9,12-tetraen-1-ylthio)butanamido)ethyl)-2-hydroxybenzamide;

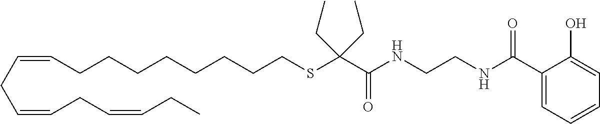 [SMILES: CC/C=C\C/C=C\C/C=C\CCCCCCCCSC(CC)(CC)C(=O)NCCNC(=O)c1ccccc1O]

N-(2-(2-ethyl-2-((9Z,12Z,15Z)-octadeca-9,12,15-trien-1-ylthio)butanamido)ethyl)-2-hydroxybenzamide;

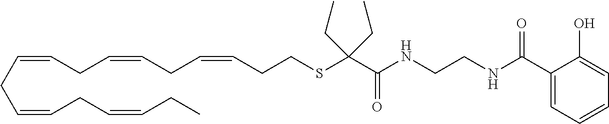 [SMILES: CC/C=C\C/C=C\C/C=C\C/C=C\C/C=C\CCSC(CC)(CC)C(=O)NCCNC(=O)c1ccccc1O]

N-(2-(2-ethyl-2-((3Z,6Z,9Z,12Z,15Z)-octadeca-3,6,9,12,15-pentaen-1-ylthio)butanamido)ethyl)-2-hydroxybenzamide;

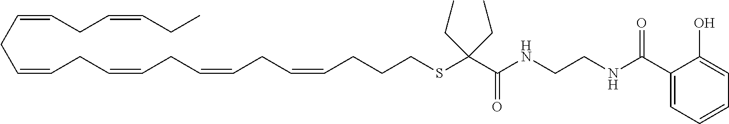 [SMILES: CC/C=C\C/C=C\C/C=C\C/C=C\C/C=C\C/C=C\CCCSC(CC)(CC)C(=O)NCCNC(=O)c1ccccc1O]

N-(2-(2-((4Z,7Z,10Z,13Z,16Z,19Z)-docosa-4,7,10,13,16,19-hexaen-1-ylthio)-2-ethylbutanamido)ethyl)-2-hydroxybenzamide;

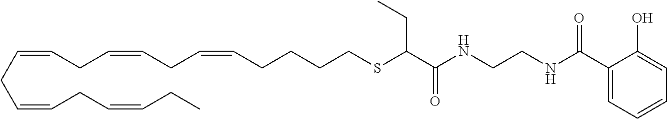 [SMILES: CC/C=C\C/C=C\C/C=C\C/C=C\C/C=C\CCCCSC(CC)C(=O)NCCNC(=O)c1ccccc1O]

2-hydroxy-N-(2-(2-((5Z,8Z,11Z,14Z,17Z)-icosa-5,8,11,14,17-pentaen-1-ylthio)butanamido)ethyl)benzamide;

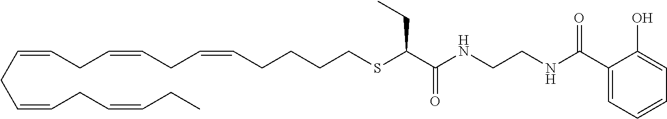 [SMILES: CC/C=C\C/C=C\C/C=C\C/C=C\C/C=C\CCCCS[C@@H](CC)C(=O)NCCNC(=O)c1ccccc1O]

2-hydroxy-N-(2-((S)-2-((5Z,8Z,11Z,14Z,17Z)-icosa-5,8,11,14,17-pentaen-1-ylthio)butanamido)ethyl)benzamide;

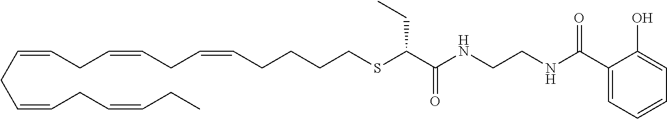 [SMILES: CC/C=C\C/C=C\C/C=C\C/C=C\C/C=C\CCCCS[C@H](CC)C(=O)NCCNC(=O)c1ccccc1O]

2-hydroxy-N-(2-((R)-2-((5Z,8Z,11Z,14Z,17Z)-icosa-5,8,11,14,17-pentaen-1-ylthio)butanamido)ethyl)benzamide;

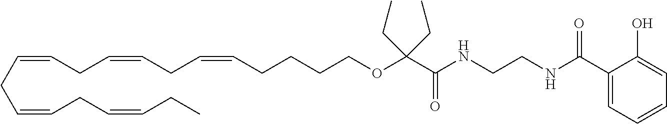 [SMILES: CC/C=C\C/C=C\C/C=C\C/C=C\C/C=C\CCCCOC(CC)(CC)C(=O)NCCNC(=O)c1ccccc1O]

N-(2-(2-ethyl-2-((5Z,8Z,11Z,14Z,17Z)-icosa-5,8,11,14,17-pentaen-1-yloxy)butanamido)ethyl)-2-hydroxybenzamide;

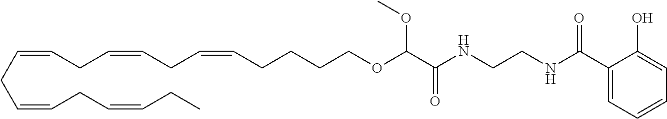 [SMILES: CC/C=C\C/C=C\C/C=C\C/C=C\C/C=C\CCCCOC(OC)C(=O)NCCNC(=O)c1ccccc1O]

2-hydroxy-N-(2-(2-((5Z,8Z,11Z,14Z,17Z)-icosa-5,8,11,14,17-pentaen-1-yloxy)-2-methoxyacetamido)ethyl)benzamide;

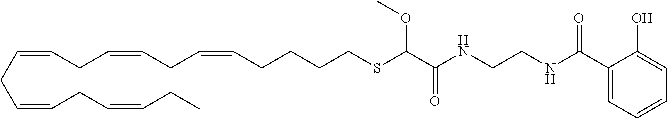 [SMILES: CC/C=C\C/C=C\C/C=C\C/C=C\C/C=C\CCCCSC(OC)C(=O)NCCNC(=O)c1ccccc1O]

2-hydroxy-N-(2-(2-((5Z,8Z,11Z,14Z,17Z)-icosa-5,8,11,14,17-pentaen-1-ylthio)-2-methoxyacetamido)ethyl)benzamide;

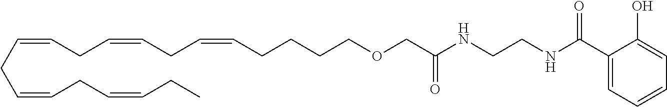 [SMILES: CC/C=C\C/C=C\C/C=C\C/C=C\C/C=C\CCCCOCC(=O)NCCNC(=O)c1ccccc1O]

2-hydroxy-N-(2-(2-((5Z,8Z,11Z,14Z,17Z)-icosa-5,8,11,14,17-pentaen-1-yloxy)acetamido)ethyl)benzamide; and

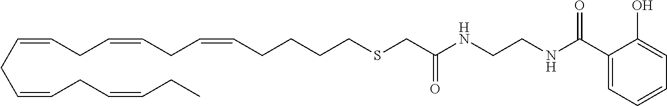 [SMILES: CC/C=C\C/C=C\C/C=C\C/C=C\C/C=C\CCCCSCC(=O)NCCNC(=O)c1ccccc1O]

2-hydroxy-N-(2-(2-((5Z,8Z,11Z,14Z,17Z)-icosa-5,8,11,14,17-pentaen-1-ylthio)acetamido)ethyl)benzamide.

Category D:

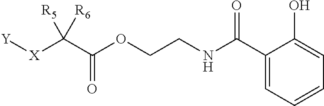

Examples:

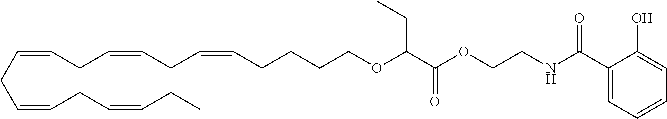 [SMILES: CC/C=C\C/C=C\C/C=C\C/C=C\C/C=C\CCCCOC(CC)C(=O)OCCNC(=O)c1ccccc1O]

2-(2-hydroxybenzamido)ethyl 2-((5Z,8Z,11Z,14Z,17Z)-icosa-5,8,11,14,17-pentaen-1-yloxy)butanoate;

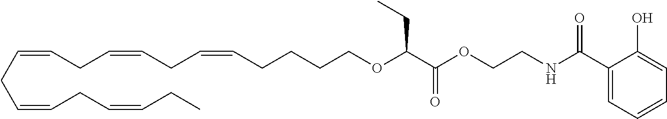 [SMILES: CC/C=C\C/C=C\C/C=C\C/C=C\C/C=C\CCCCO[C@@H](CC)C(=O)OCCNC(=O)c1ccccc1O]

(S)-2-(2-hydroxybenzamido)ethyl 2-((5Z,8Z,11Z,14Z,17Z)-icosa-5,8,11,14,17-pentaen-1-yloxy)butanoate;

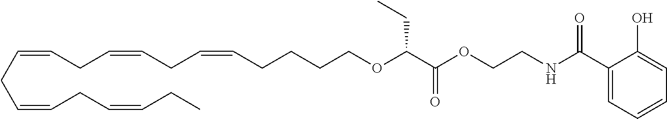 [SMILES: CC/C=C\C/C=C\C/C=C\C/C=C\C/C=C\CCCCO[C@H](CC)C(=O)OCCNC(=O)c1ccccc1O]

(R)-2-(2-hydroxybenzamido)ethyl 2-((5Z,8Z,11Z,14Z,17Z)-icosa-5,8,11,14,17-pentaen-1-yloxy)butanoate;

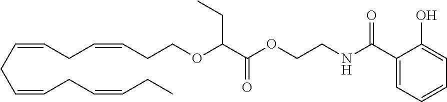 [SMILES: CC/C=C\C/C=C\C/C=C\C/C=C\CCOC(CC)C(=O)OCCNC(=O)c1ccccc1O]

2-(2-hydroxybenzamido)ethyl 2-((3Z,6Z,9Z,12Z)-pentadeca-3,6,9,12-tetraen-1-yloxy)butanoate;

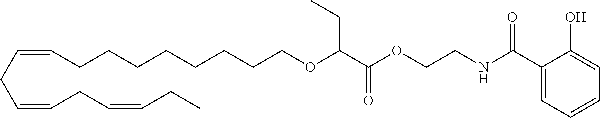 [SMILES: CC/C=C\C/C=C\C/C=C\CCCCCCCCOC(CC)C(=O)OCCNC(=O)c1ccccc1O]

2-(2-hydroxybenzamido)ethyl 2-((9Z,12Z,15Z)-octadeca-9,12,15-trien-1-yloxy)butanoate;

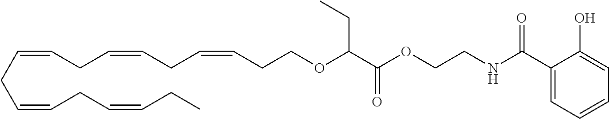 [SMILES: CC/C=C\C/C=C\C/C=C\C/C=C\C/C=C\CCOC(CC)C(=O)OCCNC(=O)c1ccccc1O]

2-(2-hydroxybenzamido)ethyl 2-((3Z,6Z,9Z,12Z,15Z)-octadeca-3,6,9,12,15-pentaen-1-yloxy)butanoate;

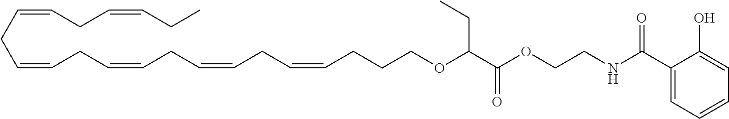 [SMILES: CC/C=C\C/C=C\C/C=C\C/C=C\C/C=C\C/C=C\CCCOC(CC)C(=O)OCCNC(=O)c1ccccc1O]

2-(2-hydroxybenzamido)ethyl 2-((4Z,7Z,10Z,13Z,16Z,19Z)-docosa-4,7,10,13,16,19-hexaen-1-yloxy)butanoate;

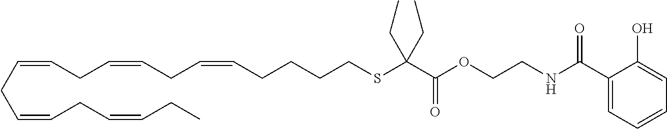 [SMILES: CC/C=C\C/C=C\C/C=C\C/C=C\C/C=C\CCCCSC(CC)(CC)C(=O)OCCNC(=O)c1ccccc1O]

2-(2-hydroxybenzamido)ethyl 2-ethyl-2-((5Z,8Z,11Z,14Z,17Z)-icosa-5,8,11,14,17-pentaen-1-ylthio)butanoate;

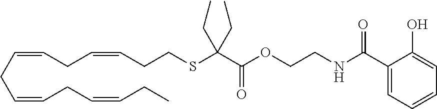 [SMILES: CC/C=C\C/C=C\C/C=C\C/C=C\CCSC(CC)(CC)C(=O)OCCNC(=O)c1ccccc1O]

2-(2-hydroxybenzamido)ethyl 2-ethyl-2-((3Z,6Z,9Z,12Z)-pentadeca-3,6,9,12-tetraen-1-ylthio)butanoate;

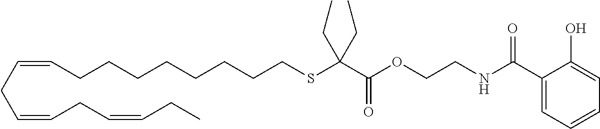 [SMILES: CC/C=C\C/C=C\C/C=C\CCCCCCCCSC(CC)(CC)C(=O)OCCNC(=O)c1ccccc1O]

2-(2-hydroxybenzamido)ethyl 2-ethyl-2-((9Z,12Z,15Z)-octadeca-9,12,15-trien-1-ylthio)butanoate;

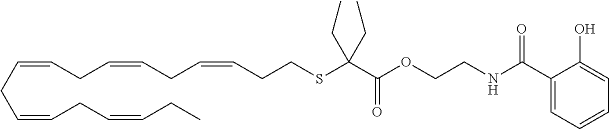 [SMILES: CC/C=C\C/C=C\C/C=C\C/C=C\C/C=C\CCSC(CC)(CC)C(=O)OCCNC(=O)c1ccccc1O]

2-(2-hydroxybenzamido)ethyl 2-ethyl-2-((3Z,6Z,9Z,12Z,15Z)-octadeca-3,6,9,12,15-pentaen-1-ylthio)butanoate;

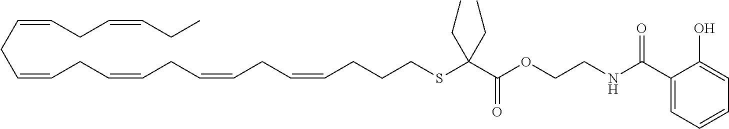 [SMILES: CC/C=C\C/C=C\C/C=C\C/C=C\C/C=C\C/C=C\CCCSC(CC)(CC)C(=O)OCCNC(=O)c1ccccc1O]

2-(2-hydroxybenzamido)ethyl 2-((4Z,7Z,10Z,13Z,16Z,19Z)-docosa-4,7,10,13,16,19-hexaen-1-ylthio)-2-ethylbutanoate;

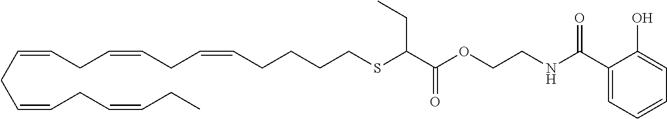 [SMILES: CC/C=C\C/C=C\C/C=C\C/C=C\C/C=C\CCCCSC(CC)C(=O)OCCNC(=O)c1ccccc1O]

2-(2-hydroxybenzamido)ethyl 2-((5Z,8Z,11Z,14Z,17Z)-icosa-5,8,11,14,17-pentaen-1-ylthio)butanoate;

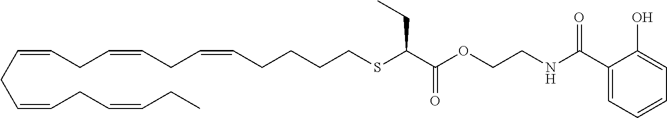 [SMILES: CC/C=C\C/C=C\C/C=C\C/C=C\C/C=C\CCCCS[C@@H](CC)C(=O)OCCNC(=O)c1ccccc1O]

(S)-2-(2-hydroxybenzamido)ethyl 2-((5Z,8Z,11Z,14Z,17Z)-icosa-5,8,11,14,17-pentaen-1-ylthio)butanoate;

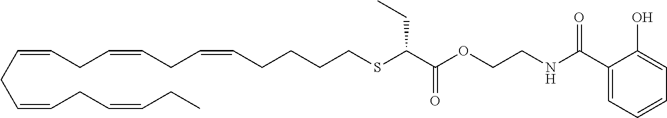 [SMILES: CC/C=C\C/C=C\C/C=C\C/C=C\C/C=C\CCCCS[C@H](CC)C(=O)OCCNC(=O)c1ccccc1O]

(R)-2-(2-hydroxybenzamido)ethyl 2-((5Z,8Z,11Z,14Z,17Z)-icosa-5,8,11,14,17-pentaen-1-ylthio)butanoate;

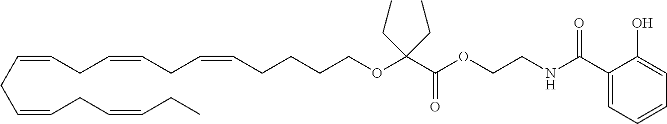 [SMILES: CC/C=C\C/C=C\C/C=C\C/C=C\C/C=C\CCCCOC(CC)(CC)C(=O)OCCNC(=O)c1ccccc1O]

2-(2-hydroxybenzamido)ethyl 2-ethyl-2-((5Z,8Z,11Z,14Z,17Z)-icosa-5,8,11,14,17-pentaen-1-yloxy)butanoate;

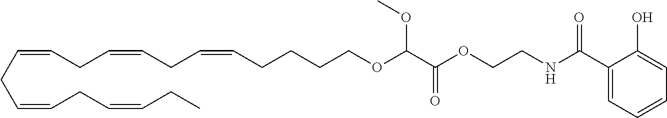 [SMILES: CC/C=C\C/C=C\C/C=C\C/C=C\C/C=C\CCCCOC(OC)C(=O)OCCNC(=O)c1ccccc1O]

2-(2-hydroxybenzamido)ethyl 2-((5Z,8Z,11Z,14Z,17Z)-icosa-5,8,11,14,17-pentaen-1-yloxy)-2-methoxyacetate;

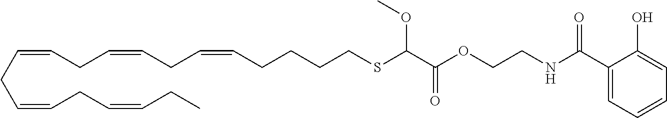 [SMILES: CC/C=C\C/C=C\C/C=C\C/C=C\C/C=C\CCCCSC(OC)C(=O)OCCNC(=O)c1ccccc1O]

2-(2-hydroxybenzamido)ethyl 2-((5Z,8Z,11Z,14Z,17Z)-icosa-5,8,11,14,17-pentaen-1-ylthio)-2-methoxyacetate;

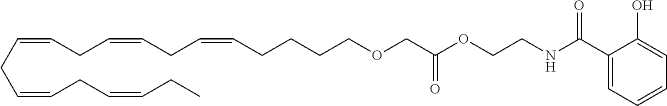 [SMILES: CC/C=C\C/C=C\C/C=C\C/C=C\C/C=C\CCCCOCC(=O)OCCNC(=O)c1ccccc1O]

2-(2-hydroxybenzamido)ethyl 2-((5Z,8Z,11Z,14Z,17Z)-icosa-5,8,11,14,17-pentaen-1-yloxy)acetate; and

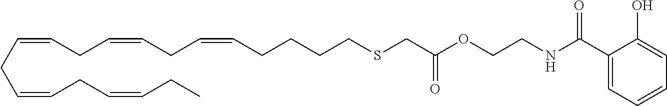 [SMILES: CC/C=C\C/C=C\C/C=C\C/C=C\C/C=C\CCCCSCC(=O)OCCNC(=O)c1ccccc1O]

2-(2-hydroxybenzamido)ethyl 2-((5Z,8Z,11Z,14Z,17Z)-icosa-5,8,11,14,17-pentaen-1-ylthio)acetate.

Category E:

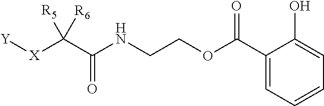

Examples:

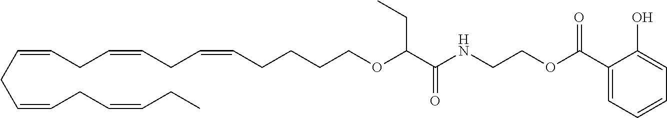 [SMILES: CC/C=C\C/C=C\C/C=C\C/C=C\C/C=C\CCCCOC(CC)C(=O)NCCOC(=O)c1ccccc1O]

2-(2-((5Z,8Z,11Z,14Z,17Z)-icosa-5,8,11,14,17-pentaen-1-yloxy)butanamido)ethyl 2-hydroxybenzoate;

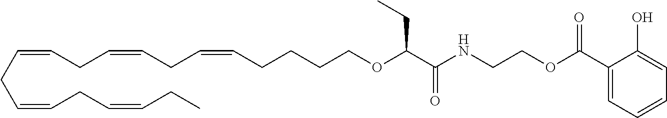 [SMILES: CC/C=C\C/C=C\C/C=C\C/C=C\C/C=C\CCCCO[C@@H](CC)C(=O)NCCOC(=O)c1ccccc1O]

2-((S)-2-((5Z,8Z,11Z,14Z,17Z)-icosa-5,8,11,14,17-pentaen-1-yloxy)butanamido)ethyl 2-hydroxybenzoate;

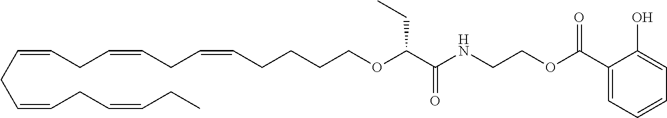 [SMILES: CC/C=C\C/C=C\C/C=C\C/C=C\C/C=C\CCCCO[C@H](CC)C(=O)NCCOC(=O)c1ccccc1O]

2-((R)-2-((5Z,8Z,11Z,14Z,17Z)-icosa-5,8,11,14,17-pentaen-1-yloxy)butanamido)ethyl 2-hydroxybenzoate;

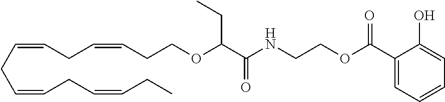 [SMILES: CC/C=C\C/C=C\C/C=C\C/C=C\CCOC(CC)C(=O)NCCOC(=O)c1ccccc1O]

2-(2-((3Z,6Z,9Z,12Z)-pentadeca-3,6,9,12-tetraen-1-yloxy)butanamido)ethyl 2-hydroxybenzoate;

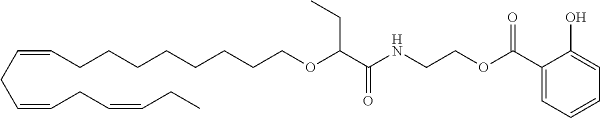 [SMILES: CC/C=C\C/C=C\C/C=C\CCCCCCCCOC(CC)C(=O)NCCOC(=O)c1ccccc1O]

2-(2-((9Z,12Z,15Z)-octadeca-9,12,15-trien-1-yloxy)butanamido)ethyl 2-hydroxybenzoate;

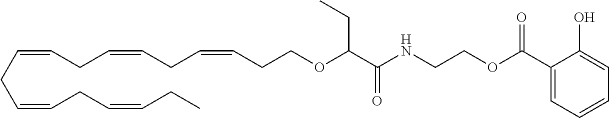 [SMILES: CC/C=C\C/C=C\C/C=C\C/C=C\C/C=C\CCOC(CC)C(=O)NCCOC(=O)c1ccccc1O]

2-(2-((3Z,6Z,9Z,12Z,15Z)-octadeca-3,6,9,12,15-pentaen-1-yloxy)butanamido)ethyl 2-hydroxybenzoate;

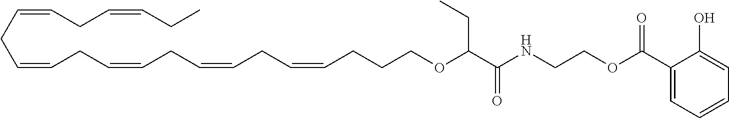 [SMILES: CC/C=C\C/C=C\C/C=C\C/C=C\C/C=C\C/C=C\CCCOC(CC)C(=O)NCCOC(=O)c1ccccc1O]

2-(2-((4Z,7Z,10Z,13Z,16Z,19Z)-docosa-4,7,10,13,16,19-hexaen-1-yloxy)butanamido)ethyl 2-hydroxybenzoate;

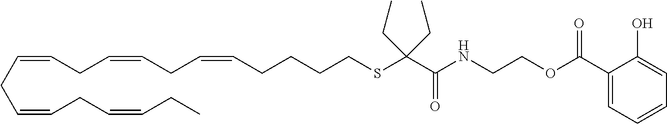 [SMILES: CC/C=C\C/C=C\C/C=C\C/C=C\C/C=C\CCCCSC(CC)(CC)C(=O)NCCOC(=O)c1ccccc1O]

2-(2-ethyl-2-((5Z,8Z,11Z,14Z,17Z)-icosa-5,8,11,14,17-pentaen-1-ylthio)butanamido)ethyl 2-hydroxybenzoate;

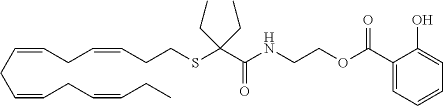 [SMILES: CC/C=C\C/C=C\C/C=C\C/C=C\CCSC(CC)(CC)C(=O)NCCOC(=O)c1ccccc1O]

2-(2-ethyl-2-((3Z,6Z,9Z,12Z)-pentadeca-3,6,9,12-tetraen-1-ylthio)butanamido)ethyl 2-hydroxybenzoate;

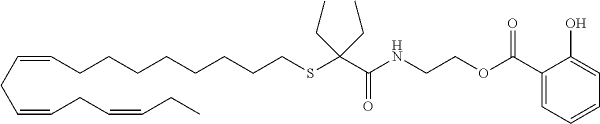 [SMILES: CC/C=C\C/C=C\C/C=C\CCCCCCCCSC(CC)(CC)C(=O)NCCOC(=O)c1ccccc1O]

2-(2-ethyl-2-((9Z,12Z,15Z)-octadeca-9,12,15-trien-1-ylthio)butanamido)ethyl 2-hydroxybenzoate;

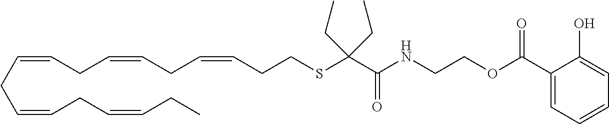 [SMILES: CC/C=C\C/C=C\C/C=C\C/C=C\C/C=C\CCSC(CC)(CC)C(=O)NCCOC(=O)c1ccccc1O]

2-(2-ethyl-2-((3Z,6Z,9Z,12Z,15Z)-octadeca-3,6,9,12,15-pentaen-1-ylthio)butanamido)ethyl 2-hydroxybenzoate;

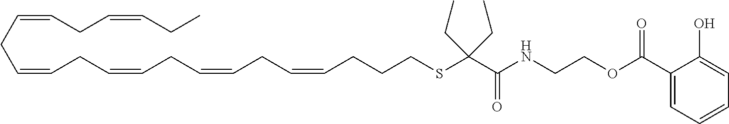 [SMILES: CC/C=C\C/C=C\C/C=C\C/C=C\C/C=C\C/C=C\CCCSC(CC)(CC)C(=O)NCCOC(=O)c1ccccc1O]

2-(2-((4Z,7Z,10Z,13Z,16Z,19Z)-docosa-4,7,10,13,16,19-hexaen-1-ylthio)-2-ethylbutanamido)ethyl 2-hydroxybenzoate;

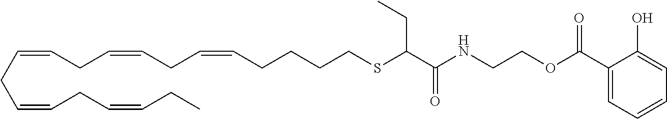 [SMILES: CC/C=C\C/C=C\C/C=C\C/C=C\C/C=C\CCCCSC(CC)C(=O)NCCOC(=O)c1ccccc1O]

2-(2-((5Z,8Z,11Z,14Z,17Z)-icosa-5,8,11,14,17-pentaen-1-ylthio)butanamido)ethyl 2-hydroxybenzoate;

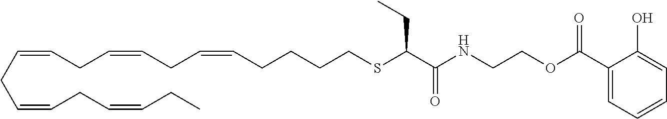 [SMILES: CC/C=C\C/C=C\C/C=C\C/C=C\C/C=C\CCCCS[C@@H](CC)C(=O)NCCOC(=O)c1ccccc1O]

2-((S)-2-((5Z,8Z,11Z,14Z,17Z)-icosa-5,8,11,14,17-pentaen-1-ylthio)butanamido)ethyl 2-hydroxybenzoate;

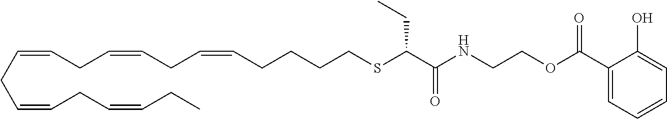 [SMILES: CC/C=C\C/C=C\C/C=C\C/C=C\C/C=C\CCCCS[C@H](CC)C(=O)NCCOC(=O)c1ccccc1O]

2-((R)-2-((5Z,8Z,11Z,14Z,17Z)-icosa-5,8,11,14,17-pentaen-1-ylthio)butanamido)ethyl 2-hydroxybenzoate;

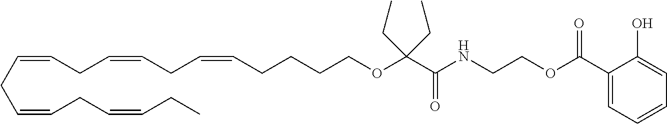 [SMILES: CC/C=C\C/C=C\C/C=C\C/C=C\C/C=C\CCCCOC(CC)(CC)C(=O)NCCOC(=O)c1ccccc1O]

2-(2-ethyl-2-((5Z,8Z,11Z,14Z,17Z)-icosa-5,8,11,14,17-pentaen-1-yloxy)butanamido)ethyl 2-hydroxybenzoate;

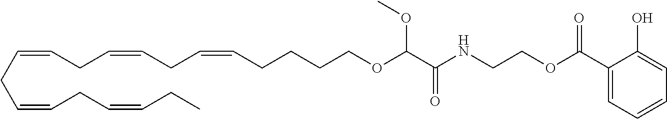 [SMILES: CC/C=C\C/C=C\C/C=C\C/C=C\C/C=C\CCCCOC(OC)C(=O)NCCOC(=O)c1ccccc1O]

2-(2-((5Z,8Z,11Z,14Z,17Z)-icosa-5,8,11,14,17-pentaen-1-yloxy)-2-methoxyacetamido)ethyl 2-hydroxybenzoate;

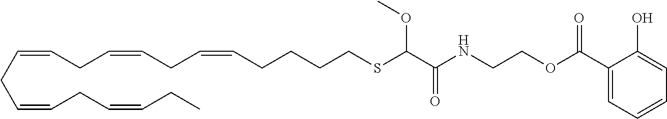 [SMILES: CC/C=C\C/C=C\C/C=C\C/C=C\C/C=C\CCCCSC(OC)C(=O)NCCOC(=O)c1ccccc1O]

2-(2-((5Z,8Z,11Z,14Z,17Z)-icosa-5,8,11,14,17-pentaen-1-ylthio)-2-methoxyacetamido)ethyl 2-hydroxybenzoate;

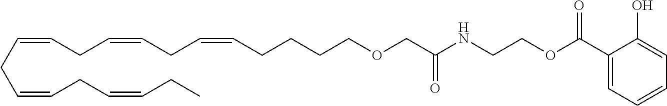 [SMILES: CC/C=C\C/C=C\C/C=C\C/C=C\C/C=C\CCCCOCC(=O)NCCOC(=O)c1ccccc1O]

2-(2-((5Z,8Z,11Z,14Z,17Z)-icosa-5,8,11,14,17-pentaen-1-yloxy)acetamido)ethyl 2-hydroxybenzoate; and

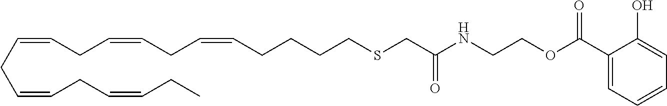 [SMILES: CC/C=C\C/C=C\C/C=C\C/C=C\C/C=C\CCCCSCC(=O)NCCOC(=O)c1ccccc1O]

2-(2-((5Z,8Z,11Z,14Z,17Z)-icosa-5,8,11,14,17-pentaen-1-ylthio)acetamido)ethyl 2-hydroxybenzoate.

Category F:

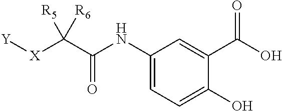

Examples:

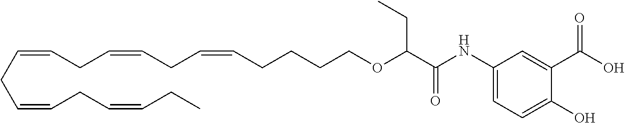 [SMILES: CC/C=C\C/C=C\C/C=C\C/C=C\C/C=C\CCCCOC(CC)C(=O)Nc1ccc(O)c(C(=O)O)c1]

2-hydroxy-5-(2-((5Z,8Z,11Z,14Z,17Z)-icosa-5,8,11,14,17-pentaen-1-yloxy)butanamido)benzoic acid;

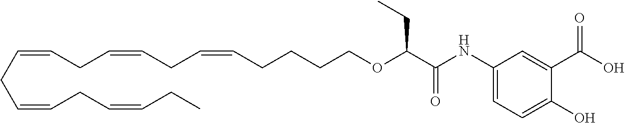 [SMILES: CC/C=C\C/C=C\C/C=C\C/C=C\C/C=C\CCCCO[C@@H](CC)C(=O)Nc1ccc(O)c(C(=O)O)c1]

2-hydroxy-5-((S)-2-((5Z,8Z,11Z,14Z,17Z)-icosa-5,8,11,14,17-pentaen-1-yloxy)butanamido)benzoic acid;

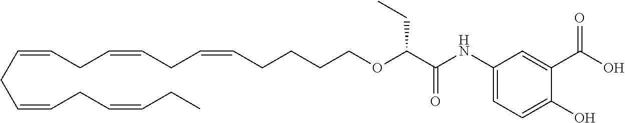 [SMILES: CC/C=C\C/C=C\C/C=C\C/C=C\C/C=C\CCCCO[C@H](CC)C(=O)Nc1ccc(O)c(C(=O)O)c1]

2-hydroxy-5-((R)-2-((5Z,8Z,11Z,14Z,17Z)-icosa-5,8,11,14,17-pentaen-1-yloxy)butanamido)benzoic acid;

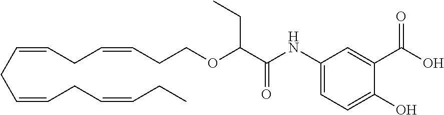 [SMILES: CC/C=C\C/C=C\C/C=C\C/C=C\CCOC(CC)C(=O)Nc1ccc(O)c(C(=O)O)c1]

2-hydroxy-5-(2-((3Z,6Z,9Z,12Z)-pentadeca-3,6,9,12-tetraen-1-yloxy)butanamido)benzoic acid;

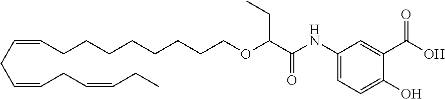 [SMILES: CC/C=C\C/C=C\C/C=C\CCCCCCCCOC(CC)C(=O)Nc1ccc(O)c(C(=O)O)c1]

2-hydroxy-5-(2-((9Z,12Z,15Z)-octadeca-9,12,15-trien-1-yloxy)butanamido)benzoic acid;

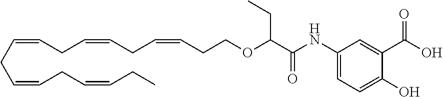 [SMILES: CC/C=C\C/C=C\C/C=C\C/C=C\C/C=C\CCOC(CC)C(=O)Nc1ccc(O)c(C(=O)O)c1]

2-hydroxy-5-(2-((3Z,6Z,9Z,12Z,15Z)-octadeca-3,6,9,12,15-pentaen-1-yloxy)butanamido)benzoic acid;

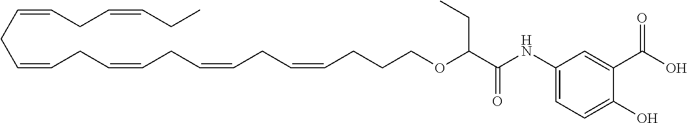 [SMILES: CC/C=C\C/C=C\C/C=C\C/C=C\C/C=C\C/C=C\CCCOC(CC)C(=O)Nc1ccc(O)c(C(=O)O)c1]

5-(2-((4Z,7Z,10Z,13Z,16Z,19Z)-docosa-4,7,10,13,16,19-hexaen-1-yloxy)butanamido)-2-hydroxybenzoic acid;

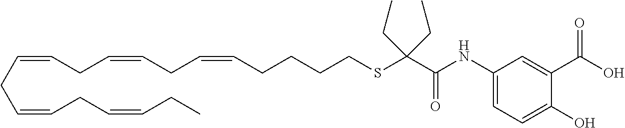 [SMILES: CC/C=C\C/C=C\C/C=C\C/C=C\C/C=C\CCCCSC(CC)(CC)C(=O)Nc1ccc(O)c(C(=O)O)c1]

5-(2-ethyl-2-((5Z,8Z,11Z,14Z,17Z)-icosa-5,8,11,14,17-pentaen-1-ylthio)butanamido)-2-hydroxybenzoic acid;

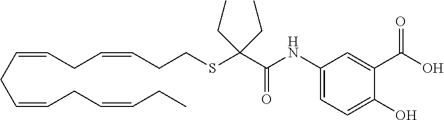 [SMILES: CC/C=C\C/C=C\C/C=C\C/C=C\CCSC(CC)(CC)C(=O)Nc1ccc(O)c(C(=O)O)c1]

5-(2-ethyl-2-((3Z,6Z,9Z,12Z)-pentadeca-3,6,9,12-tetraen-1-ylthio)butanamido)-2-hydroxybenzoic acid;

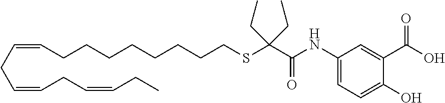 [SMILES: CC/C=C\C/C=C\C/C=C\CCCCCCCCSC(CC)(CC)C(=O)Nc1ccc(O)c(C(=O)O)c1]

5-(2-ethyl-2-((9Z,12Z,15Z)-octadeca-9,12,15-trien-1-ylthio)butanamido)-2-hydroxybenzoic acid;

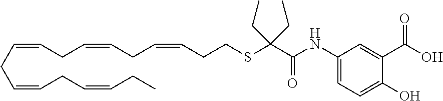 [SMILES: CC/C=C\C/C=C\C/C=C\C/C=C\C/C=C\CCSC(CC)(CC)C(=O)Nc1ccc(O)c(C(=O)O)c1]

5-(2-ethyl-2-((3Z,6Z,9Z,12Z,15Z)-octadeca-3,6,9,12,15-pentaen-1-ylthio)butanamido)-2-hydroxybenzoic acid;

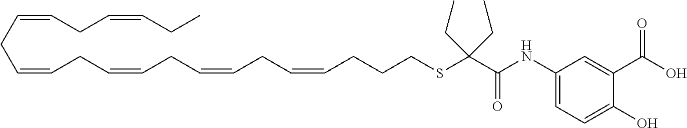 [SMILES: CC/C=C\C/C=C\C/C=C\C/C=C\C/C=C\C/C=C\CCCSC(CC)(CC)C(=O)Nc1ccc(O)c(C(=O)O)c1]

5-(2-((4Z,7Z,10Z,13Z,16Z,19Z)-docosa-4,7,10,13,16,19-hexaen-1-ylthio)-2-ethylbutanamido)-2-hydroxybenzoic acid;

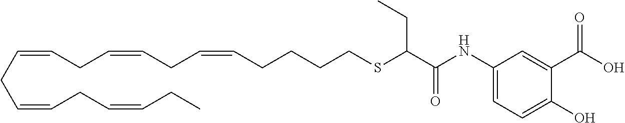 [SMILES: CC/C=C\C/C=C\C/C=C\C/C=C\C/C=C\CCCCSC(CC)C(=O)Nc1ccc(O)c(C(=O)O)c1]

2-hydroxy-5-(2-((5Z,8Z,11Z,14Z,17Z)-icosa-5,8,11,14,17-pentaen-1-ylthio)butanamido)benzoic acid;

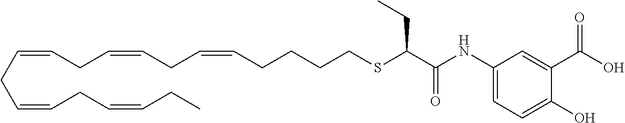 [SMILES: CC/C=C\C/C=C\C/C=C\C/C=C\C/C=C\CCCCS[C@@H](CC)C(=O)Nc1ccc(O)c(C(=O)O)c1]

2-hydroxy-5-((S)-2-((5Z,8Z,11Z,14Z,17Z)-icosa-5,8,11,14,17-pentaen-1-ylthio)butanamido)benzoic acid;

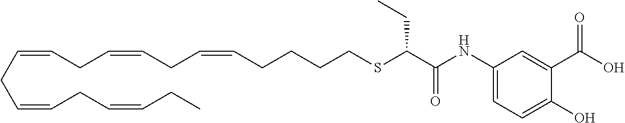 [SMILES: CC/C=C\C/C=C\C/C=C\C/C=C\C/C=C\CCCCS[C@H](CC)C(=O)Nc1ccc(O)c(C(=O)O)c1]

2-hydroxy-5-((R)-2-((5Z,8Z,11Z,14Z,17Z)-icosa-5,8,11,14,17-pentaen-1-ylthio)butanamido)benzoic acid;

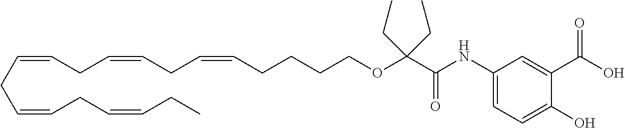 [SMILES: CC/C=C\C/C=C\C/C=C\C/C=C\C/C=C\CCCCOC(CC)(CC)C(=O)Nc1ccc(O)c(C(=O)O)c1]

5-(2-ethyl-2-((5Z,8Z,11Z,14Z,17Z)-icosa-5,8,11,14,17-pentaen-1-yloxy)butanamido)-2-hydroxybenzoic acid;

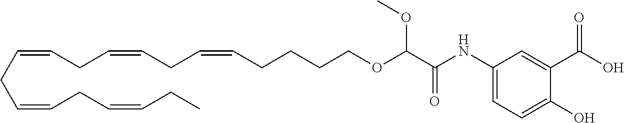 [SMILES: CC/C=C\C/C=C\C/C=C\C/C=C\C/C=C\CCCCOC(OC)C(=O)Nc1ccc(O)c(C(=O)O)c1]

2-hydroxy-5-(2-((5Z,8Z,11Z,14Z,17Z)-icosa-5,8,11,14,17-pentaen-1-yloxy)-2-methoxyacetamido)benzoic acid;

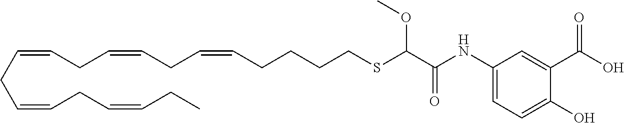 [SMILES: CC/C=C\C/C=C\C/C=C\C/C=C\C/C=C\CCCCSC(OC)C(=O)Nc1ccc(O)c(C(=O)O)c1]

2-hydroxy-5-(2-((5Z,8Z,11Z,14Z,17Z)-icosa-5,8,11,14,17-pentaen-1-ylthio)-2-methoxyacetamido)benzoic acid;

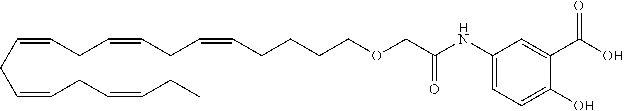 [SMILES: CC/C=C\C/C=C\C/C=C\C/C=C\C/C=C\CCCCOCC(=O)Nc1ccc(O)c(C(=O)O)c1]

2-hydroxy-5-(2-((5Z,8Z,11Z,14Z,17Z)-icosa-5,8,11,14,17-pentaen-1-yloxy)acetamido)benzoic acid; and

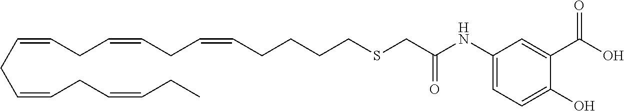 [SMILES: CC/C=C\C/C=C\C/C=C\C/C=C\C/C=C\CCCCSCC(=O)Nc1ccc(O)c(C(=O)O)c1]

2-hydroxy-5-(2-((5Z,8Z,11Z,14Z,17Z)-icosa-5,8,11,14,17-pentaen-1-ylthio)acetamido)benzoic acid.

Category G:

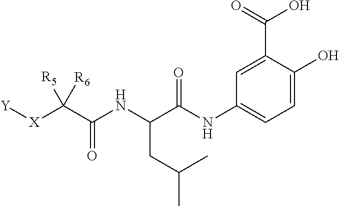

Examples:

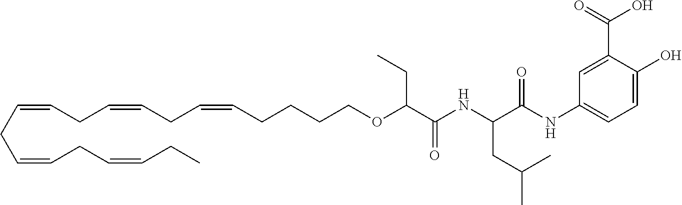 [SMILES: CC/C=C\C/C=C\C/C=C\C/C=C\C/C=C\CCCCOC(CC)C(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-yloxy)butanamido)-4-methylpentanamido)benzoic acid;

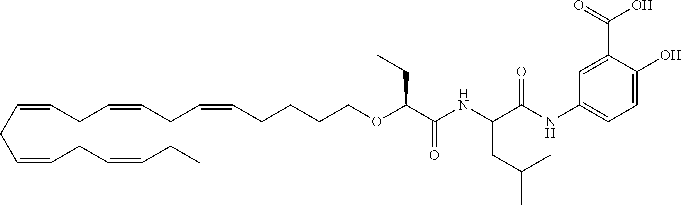 [SMILES: CC/C=C\C/C=C\C/C=C\C/C=C\C/C=C\CCCCO[C@@H](CC)C(=O)NC(CC(C)C)C(=O)Nc1ccc(O)c(C(=O)O)c1]

2-hydroxy-5-(2-((S)-2-((5Z,8Z,11Z,14Z,17Z)-icosa-5,8,11,14,17-pentaen-1-yloxy)butanamido)-4-methylpentanamido)benzoic acid;

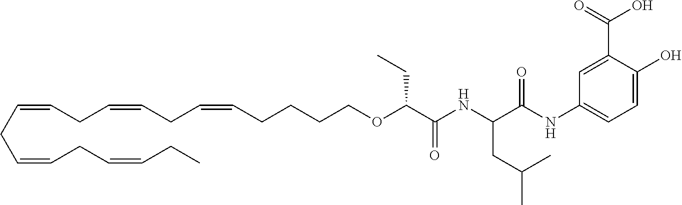 [SMILES: CC/C=C\C/C=C\C/C=C\C/C=C\C/C=C\CCCCO[C@H](CC)C(=O)NC(CC(C)C)C(=O)Nc1ccc(O)c(C(=O)O)c1]

2-hydroxy-5-(2-((R)-2-((5Z,8Z,11Z,14Z,17Z)-icosa-5,8,11,14,17-pentaen-1-yloxy)butanamido)-4-methylpentanamido)benzoic acid;

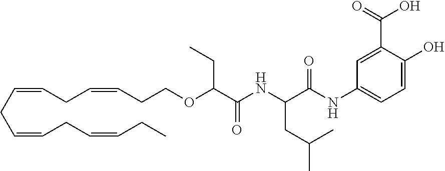 [SMILES: CC/C=C\C/C=C\C/C=C\C/C=C\CCOC(CC)C(=O)NC(CC(C)C)C(=O)Nc1ccc(O)c(C(=O)O)c1]

2-hydroxy-5-(4-methyl-2-(2-((3Z,6Z,9Z,12Z)-pentadeca-3,6,9,12-tetraen-1-yloxy)butanamido)pentanamido)benzoic acid;

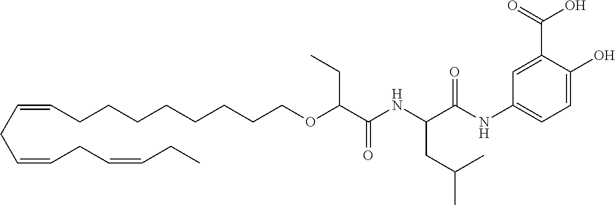 [SMILES: CC/C=C\C/C=C\C/C=C\CCCCCCCCOC(CC)C(=O)NC(CC(C)C)C(=O)Nc1ccc(O)c(C(=O)O)c1]

2-hydroxy-5-(4-methyl-2-(2-((9Z,12Z,15Z)-octadeca-9,12,15-trien-1-yloxy)butanamido)pentanamido)benzoic acid;

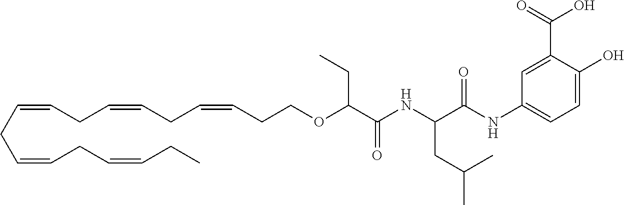 [SMILES: CC/C=C\C/C=C\C/C=C\C/C=C\C/C=C\CCOC(CC)C(=O)NC(CC(C)C)C(=O)Nc1ccc(O)c(C(=O)O)c1]

2-hydroxy-5-(4-methyl-2-(2-((3Z,6Z,9Z,12Z,15Z)-octadeca-3,6,9,12,15-pentaen-1-yloxy)butanamido)pentanamido)benzoic acid;

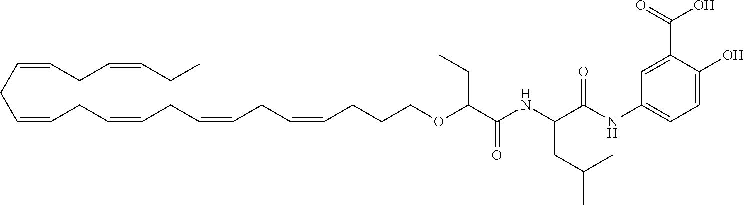 [SMILES: CC/C=C\C/C=C\C/C=C\C/C=C\C/C=C\C/C=C\CCCOC(CC)C(=O)NC(CC(C)C)C(=O)Nc1ccc(O)c(C(=O)O)c1]

5-(2-(2-((4Z,7Z,10Z,13Z,16Z,19Z)-docosa-4,7,10,13,16,19-hexaen-1-yloxy)butanamido)-4-methylpentanamido)-2-hydroxybenzoic acid;

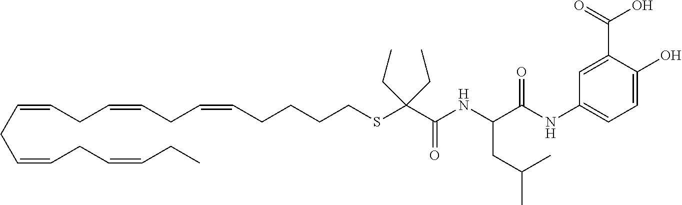 [SMILES: CC/C=C\C/C=C\C/C=C\C/C=C\C/C=C\CCCCSC(CC)(CC)C(=O)NC(CC(C)C)C(=O)Nc1ccc(O)c(C(=O)O)c1]

5-(2-(2-ethyl-2-((5Z,8Z,11Z,14Z,17Z)-icosa-5,8,11,14,17-pentaen-1-ylthio)butanamido)-4-methylpentanamido)-2-hydroxybenzoic acid;

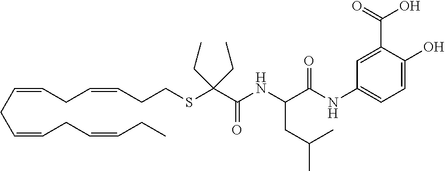 [SMILES: CC/C=C\C/C=C\C/C=C\C/C=C\CCSC(CC)(CC)C(=O)NC(CC(C)C)C(=O)Nc1ccc(O)c(C(=O)O)c1]

5-(2-(2-ethyl-2-((3Z,6Z,9Z,12Z)-pentadeca-3,6,9,12-tetraen-1-ylthio)butanamido)-4-methylpentanamido)-2-hydroxybenzoic acid;

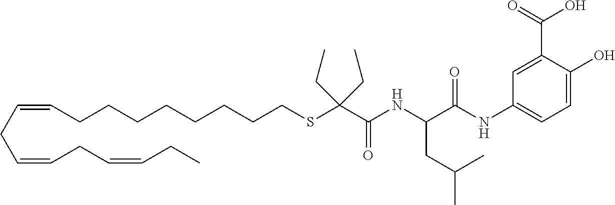 [SMILES: CC/C=C\C/C=C\C/C=C\CCCCCCCCSC(CC)(CC)C(=O)NC(CC(C)C)C(=O)Nc1ccc(O)c(C(=O)O)c1]

5-(2-(2-ethyl-2-((9Z,12Z,15Z)-octadeca-9,12,15-trien-1-ylthio)butanamido)-4-methylpentanamido)-2-hydroxybenzoic acid;

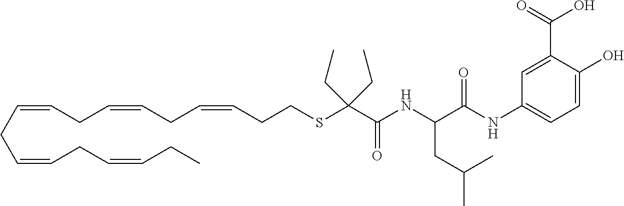 [SMILES: CC/C=C\C/C=C\C/C=C\C/C=C\C/C=C\CCSC(CC)(CC)C(=O)NC(CC(C)C)C(=O)Nc1ccc(O)c(C(=O)O)c1]

5-(2-(2-ethyl-2-((3Z,6Z,9Z,12Z,15Z)-octadeca-3,6,9,12,15-pentaen-1-ylthio)butanamido)-4-methylpentanamido)-2-hydroxybenzoic acid;

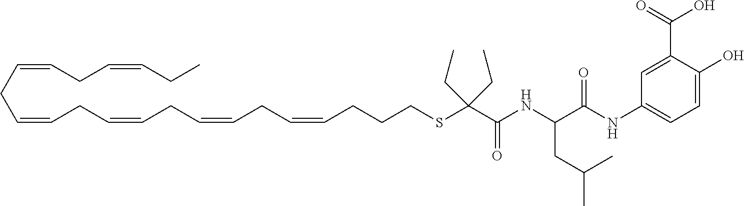 [SMILES: CC/C=C\C/C=C\C/C=C\C/C=C\C/C=C\C/C=C\CCCSC(CC)(CC)C(=O)NC(CC(C)C)C(=O)Nc1ccc(O)c(C(=O)O)c1]

5-(2-(2-((4Z,7Z,10Z,13Z,16Z,19Z)-docosa-4,7,10,13,16,19-hexaen-1-ylthio)-2-ethylbutanamido)-4-methylpentanamido)-2-hydroxybenzoic acid;

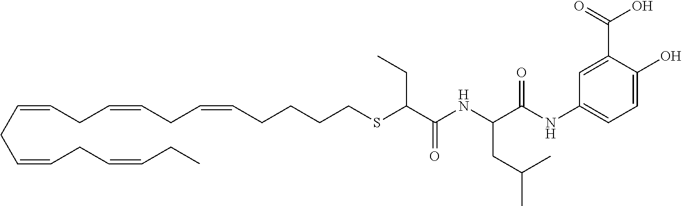 [SMILES: CC/C=C\C/C=C\C/C=C\C/C=C\C/C=C\CCCCSC(CC)C(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-ylthio)butanamido)-4-methylpentanamido)benzoic acid;

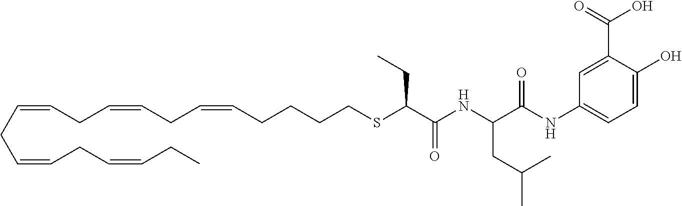 [SMILES: CC/C=C\C/C=C\C/C=C\C/C=C\C/C=C\CCCCS[C@@H](CC)C(=O)NC(CC(C)C)C(=O)Nc1ccc(O)c(C(=O)O)c1]

2-hydroxy-5-(2-((S)-2-((5Z,8Z,11Z,14Z,17Z)-icosa-5,8,11,14,17-pentaen-1-ylthio)butanamido)-4-methylpentanamido)benzoic acid;

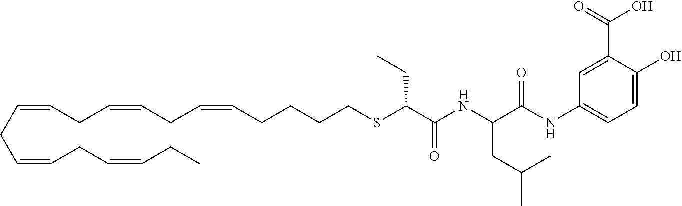 [SMILES: CC/C=C\C/C=C\C/C=C\C/C=C\C/C=C\CCCCS[C@H](CC)C(=O)NC(CC(C)C)C(=O)Nc1ccc(O)c(C(=O)O)c1]

2-hydroxy-5-(2-((R)-2-((5Z,8Z,11Z,14Z,17Z)-icosa-5,8,11,14,17-pentaen-1-ylthio)butanamido)-4-methylpentanamido)benzoic acid;

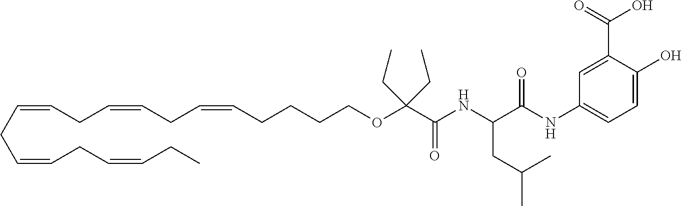 [SMILES: CC/C=C\C/C=C\C/C=C\C/C=C\C/C=C\CCCCOC(CC)(CC)C(=O)NC(CC(C)C)C(=O)Nc1ccc(O)c(C(=O)O)c1]

5-(2-(2-ethyl-2-((5Z,8Z,11Z,14Z,17Z)-icosa-5,8,11,14,17-pentaen-1-yloxy)butanamido)-4-methylpentanamido)-2-hydroxybenzoic acid;

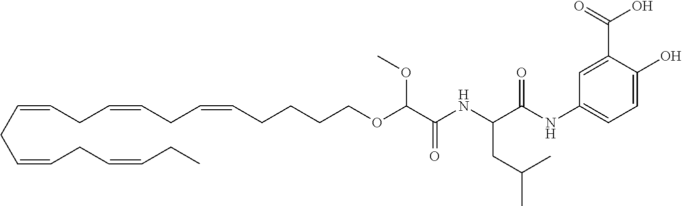 [SMILES: CC/C=C\C/C=C\C/C=C\C/C=C\C/C=C\CCCCOC(OC)C(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-yloxy)-2-methoxyacetamido)-4-methylpentanamido)benzoic acid;

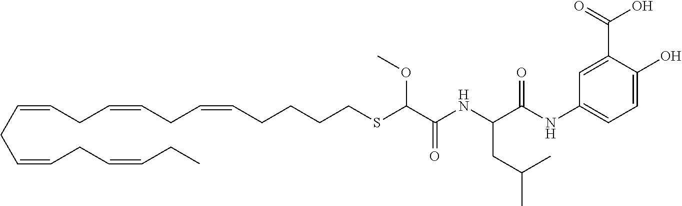 [SMILES: CC/C=C\C/C=C\C/C=C\C/C=C\C/C=C\CCCCSC(OC)C(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-ylthio)-2-methoxyacetamido)-4-methylpentanamido)benzoic acid;

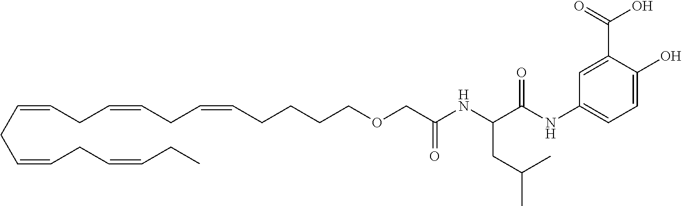 [SMILES: CC/C=C\C/C=C\C/C=C\C/C=C\C/C=C\CCCCOCC(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-yloxy)acetamido)-4-methylpentanamido)benzoic acid; and

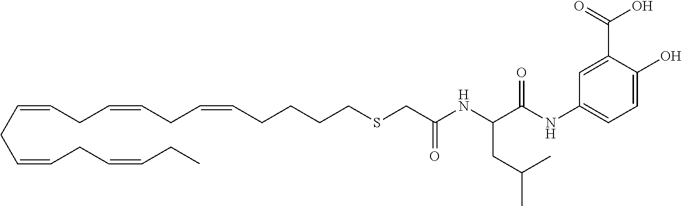 [SMILES: CC/C=C\C/C=C\C/C=C\C/C=C\C/C=C\CCCCSCC(=O)NC(CC(C)C)C(=O)Nc1ccc(O)c(C(=O)O)c1]

2-hydroxy-5-(2-(2-((5Z,8Z,11Z,14Z,17Z)-icosa-5,8,11,14,17-pentaen-1-ylthio)acetamido)-4-methylpentanamido)benzoic acid.

Category H:

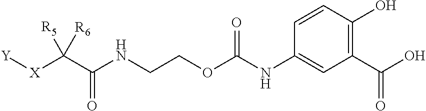

Examples:

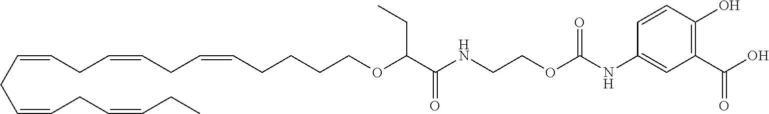 [SMILES: CC/C=C\C/C=C\C/C=C\C/C=C\C/C=C\CCCCOC(CC)C(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-yloxy)butanamido)ethoxy)carbonyl)amino)benzoic acid;

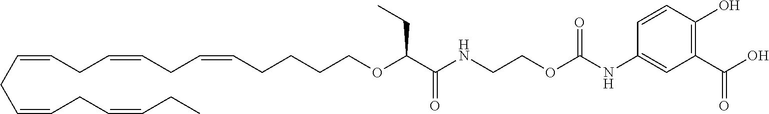 [SMILES: CC/C=C\C/C=C\C/C=C\C/C=C\C/C=C\CCCCO[C@@H](CC)C(=O)NCCOC(=O)Nc1ccc(O)c(C(=O)O)c1]

2-hydroxy-5-(((2-((S)-2-((5Z,8Z,11Z,14Z,17Z)-icosa-5,8,11,14,17-pentaen-1-yloxy)butanamido)ethoxy)carbonyl)amino)benzoic acid;

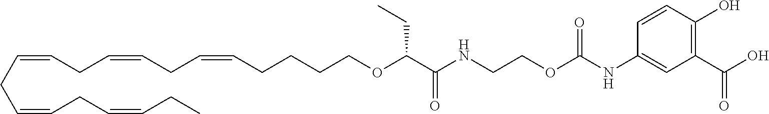 [SMILES: CC/C=C\C/C=C\C/C=C\C/C=C\C/C=C\CCCCO[C@H](CC)C(=O)NCCOC(=O)Nc1ccc(O)c(C(=O)O)c1]

2-hydroxy-5-(((2-((R)-2-((5Z,8Z,11Z,14Z,17Z)-icosa-5,8,11,14,17-pentaen-1-yloxy)butanamido)ethoxy)carbonyl)amino)benzoic acid;

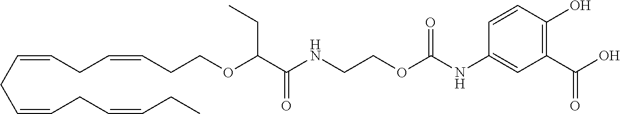 [SMILES: CC/C=C\C/C=C\C/C=C\C/C=C\CCOC(CC)C(=O)NCCOC(=O)Nc1ccc(O)c(C(=O)O)c1]

2-hydroxy-5-(((2-(2-((3Z,6Z,9Z,12Z)-pentadeca-3,6,9,12-tetraen-1-yloxy)butanamido)ethoxy)carbonyl)amino)benzoic acid;

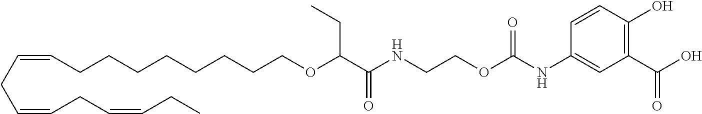 [SMILES: CC/C=C\C/C=C\C/C=C\CCCCCCCCOC(CC)C(=O)NCCOC(=O)Nc1ccc(O)c(C(=O)O)c1]

2-hydroxy-5-(((2-(2-((9Z,12Z,15Z)-octadeca-9,12,15-trien-1-yloxy)butanamido)ethoxy)carbonyl)amino)benzoic acid;

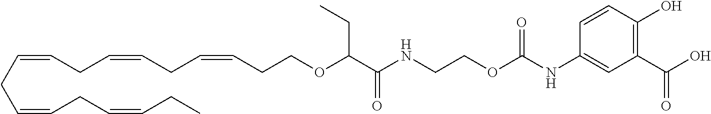 [SMILES: CC/C=C\C/C=C\C/C=C\C/C=C\C/C=C\CCOC(CC)C(=O)NCCOC(=O)Nc1ccc(O)c(C(=O)O)c1]

2-hydroxy-5-(((2-(2-((3Z,6Z,9Z,12Z,15Z)-octadeca-3,6,9,12,15-pentaen-1-yloxy)butanamido)ethoxy)carbonyl)amino)benzoic acid;

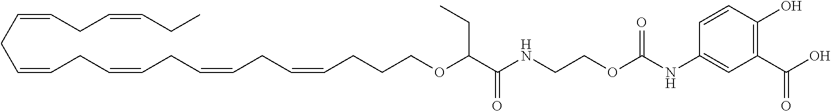 [SMILES: CC/C=C\C/C=C\C/C=C\C/C=C\C/C=C\C/C=C\CCCOC(CC)C(=O)NCCOC(=O)Nc1ccc(O)c(C(=O)O)c1]

5-(((2-(2-((4Z,7Z,10Z,13Z,16Z,19Z)-docosa-4,7,10,13,16,19-hexaen-1-yloxy)butanamido)ethoxy)carbonyl)amino)-2-hydroxybenzoic acid;

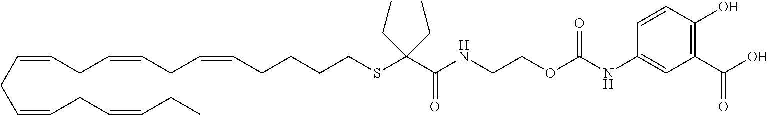 [SMILES: CC/C=C\C/C=C\C/C=C\C/C=C\C/C=C\CCCCSC(CC)(CC)C(=O)NCCOC(=O)Nc1ccc(O)c(C(=O)O)c1]

5-(((2-(2-ethyl-2-((5Z,8Z,11Z,14Z,17Z)-icosa-5,8,11,14,17-pentaen-1-ylthio)butanamido)ethoxy)carbonyl)amino)-2-hydroxybenzoic acid;

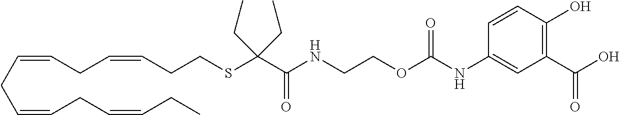 [SMILES: CC/C=C\C/C=C\C/C=C\C/C=C\CCSC(CC)(CC)C(=O)NCCOC(=O)Nc1ccc(O)c(C(=O)O)c1]

5-(((2-(2-ethyl-2-((3Z,6Z,9Z,12Z)-pentadeca-3,6,9,12-tetraen-1-ylthio)butanamido)ethoxy)carbonyl)amino)-2-hydroxybenzoic acid;

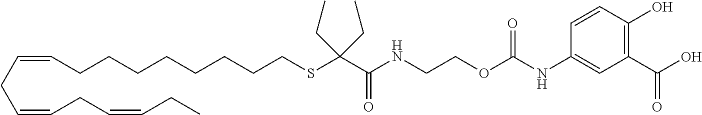 [SMILES: CC/C=C\C/C=C\C/C=C\CCCCCCCCSC(CC)(CC)C(=O)NCCOC(=O)Nc1ccc(O)c(C(=O)O)c1]

5-(((2-(2-ethyl-2-((9Z,12Z,15Z)-octadeca-9,12,15-trien-1-ylthio)butanamido)ethoxy)carbonyl)amino)-2-hydroxybenzoic acid;

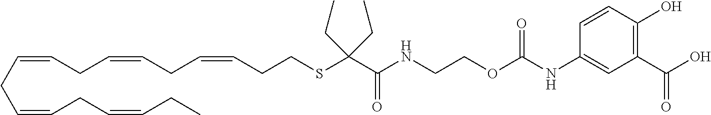 [SMILES: CC/C=C\C/C=C\C/C=C\C/C=C\C/C=C\CCSC(CC)(CC)C(=O)NCCOC(=O)Nc1ccc(O)c(C(=O)O)c1]

5-(((2-(2-ethyl-2-((3Z,6Z,9Z,12Z,15Z)-octadeca-3,6,9,12,15-pentaen-1-ylthio)butanamido)ethoxy)carbonyl)amino)-2-hydroxybenzoic acid;

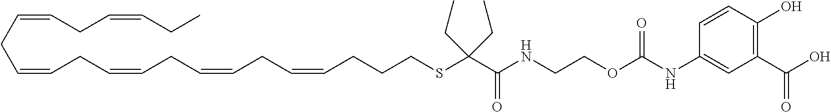 [SMILES: CC/C=C\C/C=C\C/C=C\C/C=C\C/C=C\C/C=C\CCCSC(CC)(CC)C(=O)NCCOC(=O)Nc1ccc(O)c(C(=O)O)c1]

5-(((2-(2-((4Z,7Z,10Z,13Z,16Z,19Z)-docosa-4,7,10,13,16,19-hexaen-1-ylthio)-2-ethylbutanamido)ethoxy)carbonyl)amino)-2-hydroxybenzoic acid;

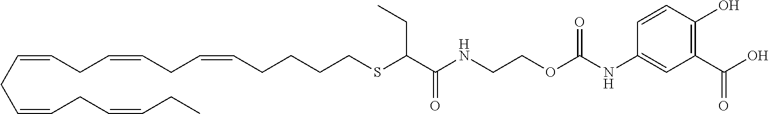 [SMILES: CC/C=C\C/C=C\C/C=C\C/C=C\C/C=C\CCCCSC(CC)C(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-ylthio)butanamido)ethoxy)carbonyl)amino)benzoic acid;

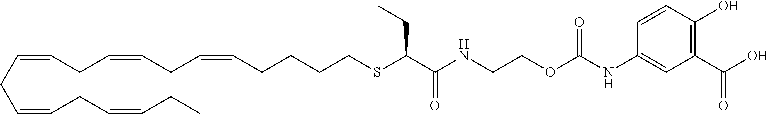 [SMILES: CC/C=C\C/C=C\C/C=C\C/C=C\C/C=C\CCCCS[C@@H](CC)C(=O)NCCOC(=O)Nc1ccc(O)c(C(=O)O)c1]

2-hydroxy-5-(((2-((S)-2-((5Z,8Z,11Z,14Z,17Z)-icosa-5,8,11,14,17-pentaen-1-ylthio)butanamido)ethoxy)carbonyl)amino)benzoic acid;

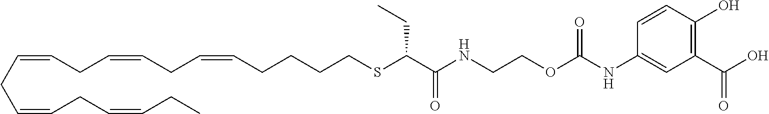 [SMILES: CC/C=C\C/C=C\C/C=C\C/C=C\C/C=C\CCCCS[C@H](CC)C(=O)NCCOC(=O)Nc1ccc(O)c(C(=O)O)c1]

2-hydroxy-5-(((2-((R)-2-((5Z,8Z,11Z,14Z,17Z)-icosa-5,8,11,14,17-pentaen-1-ylthio)butanamido)ethoxy)carbonyl)amino)benzoic acid:

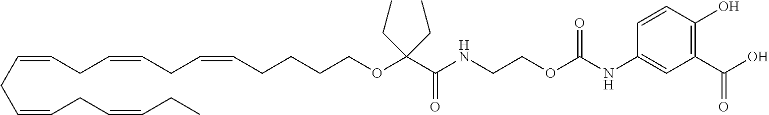 [SMILES: CC/C=C\C/C=C\C/C=C\C/C=C\C/C=C\CCCCOC(CC)(CC)C(=O)NCCOC(=O)Nc1ccc(O)c(C(=O)O)c1]

5-(((2-(2-ethyl-2-((5Z,8Z,11Z,14Z,17Z)-icosa-5,8,11,14,17-pentaen-1-yloxy)butanamido)ethoxy)carbonyl)amino)-2-hydroxybenzoic acid;

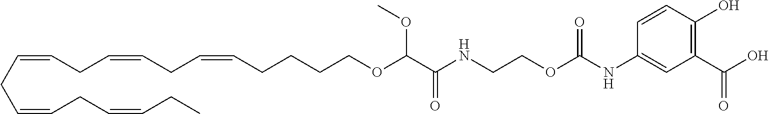 [SMILES: CC/C=C\C/C=C\C/C=C\C/C=C\C/C=C\CCCCOC(OC)C(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-yloxy)-2-methoxyacetamido)ethoxy)carbonyl)amino)benzoic acid;

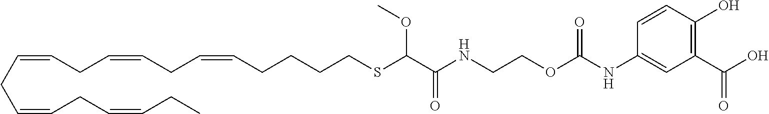 [SMILES: CC/C=C\C/C=C\C/C=C\C/C=C\C/C=C\CCCCSC(OC)C(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-ylthio)-2-methoxyacetamido)ethoxy)carbonyl)amino)benzoic acid;

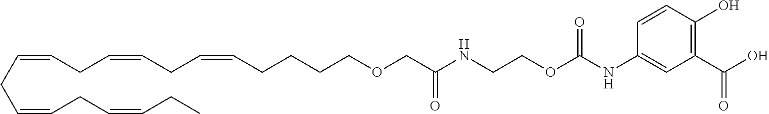 [SMILES: CC/C=C\C/C=C\C/C=C\C/C=C\C/C=C\CCCCOCC(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-yloxy)acetamido)ethoxy)carbonyl)amino)benzoic acid;

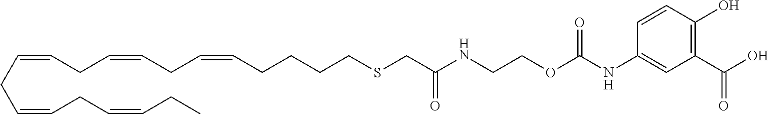 [SMILES: CC/C=C\C/C=C\C/C=C\C/C=C\C/C=C\CCCCSCC(=O)NCCOC(=O)Nc1ccc(O)c(C(=O)O)c1]

2-hydroxy-5-(((2-(2-((5Z,8Z,11Z,14Z,17Z)-icosa-5,8,11,14,17-pentaen-1-ylthio)acetamido)ethoxy)carbonyl)amino)benzoic acid;

Category I:

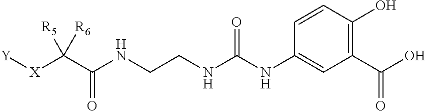

Examples:

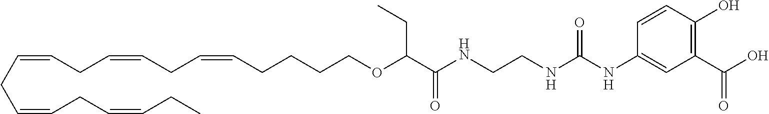 [SMILES: CC/C=C\C/C=C\C/C=C\C/C=C\C/C=C\CCCCOC(CC)C(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-yloxy)butanamido)ethyl)ureido)benzoic acid;

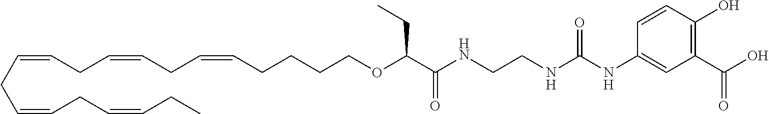 [SMILES: CC/C=C\C/C=C\C/C=C\C/C=C\C/C=C\CCCCO[C@@H](CC)C(=O)NCCNC(=O)Nc1ccc(O)c(C(=O)O)c1]

2-hydroxy-5-(3-(2-((S)-2-((5Z,8Z,11Z,14Z,17Z)-icosa-5,8,11,14,17-pentaen-1-yloxy)butanamido)ethyl)ureido)benzoic acid;

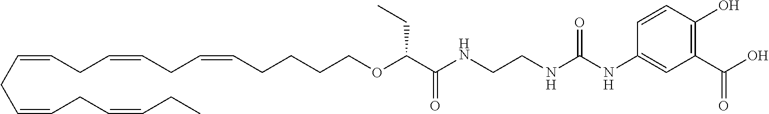 [SMILES: CC/C=C\C/C=C\C/C=C\C/C=C\C/C=C\CCCCO[C@H](CC)C(=O)NCCNC(=O)Nc1ccc(O)c(C(=O)O)c1]

2-hydroxy-5-(3-(2-((R)-2-((5Z,8Z,11Z,14Z,17Z)-icosa-5,8,11,14,17-pentaen-1-yloxy)butanamido)ethyl)ureido)benzoic acid;

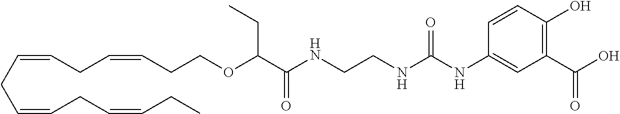 [SMILES: CC/C=C\C/C=C\C/C=C\C/C=C\CCOC(CC)C(=O)NCCNC(=O)Nc1ccc(O)c(C(=O)O)c1]

2-hydroxy-5-(3-(2-(2-((3Z,6Z,9Z,12Z)-pentadeca-3,6,9,12-tetraen-1-yloxy)butanamido)ethyl)ureido)benzoic acid;

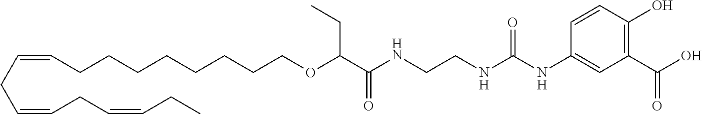 [SMILES: CC/C=C\C/C=C\C/C=C\CCCCCCCCOC(CC)C(=O)NCCNC(=O)Nc1ccc(O)c(C(=O)O)c1]

2-hydroxy-5-(3-(2-(2-((9Z,12Z,15Z)-octadeca-9,12,15-trien-1-yloxy)butanamido)ethyl)ureido)benzoic acid;

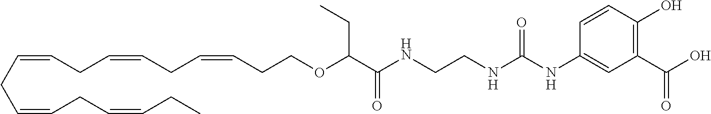 [SMILES: CC/C=C\C/C=C\C/C=C\C/C=C\C/C=C\CCOC(CC)C(=O)NCCNC(=O)Nc1ccc(O)c(C(=O)O)c1]

2-hydroxy-5-(3-(2-(2-((3Z,6Z,9Z,12Z,15Z)-octadeca-3,6,9,12,15-pentaen-1-yloxy)butanamido)ethyl)ureido)benzoic acid;

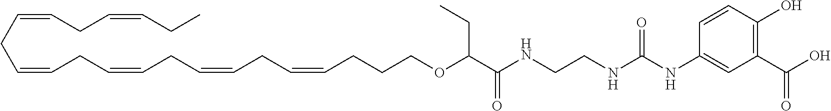 [SMILES: CC/C=C\C/C=C\C/C=C\C/C=C\C/C=C\C/C=C\CCCOC(CC)C(=O)NCCNC(=O)Nc1ccc(O)c(C(=O)O)c1]

5-(3-(2-(2-((4Z,7Z,10Z,13Z,16Z,19Z)-docosa-4,7,10,13,16,19-hexaen-1-yloxy)butanamido)ethyl)ureido)-2-hydroxybenzoic acid;

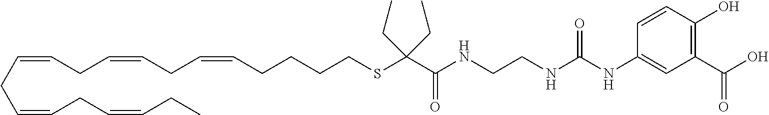 [SMILES: CC/C=C\C/C=C\C/C=C\C/C=C\C/C=C\CCCCSC(CC)(CC)C(=O)NCCNC(=O)Nc1ccc(O)c(C(=O)O)c1]

5-(3-(2-(2-ethyl-2-((5Z,8Z,11Z,14Z,17Z)-icosa-5,8,11,14,17-pentaen-1-ylthio)butanamido)ethyl)ureido)-2-hydroxybenzoic acid;

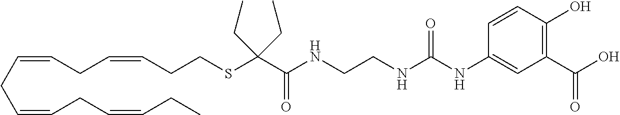 [SMILES: CC/C=C\C/C=C\C/C=C\C/C=C\CCSC(CC)(CC)C(=O)NCCNC(=O)Nc1ccc(O)c(C(=O)O)c1]

5-(3-(2-(2-ethyl-2-((3Z,6Z,9Z,12Z)-pentadeca-3,6,9,12-tetraen-1-ylthio)butanamido)ethyl)ureido)-2-hydroxybenzoic acid;

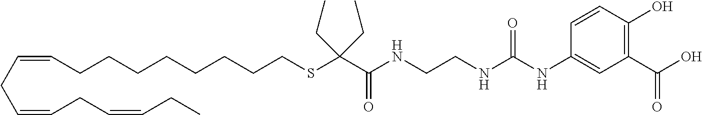 [SMILES: CC/C=C\C/C=C\C/C=C\CCCCCCCCSC(CC)(CC)C(=O)NCCNC(=O)Nc1ccc(O)c(C(=O)O)c1]

5-(3-(2-(2-ethyl-2-((9Z,12Z,15Z)-octadeca-9,12,15-trien-1-ylthio)butanamido)ethyl)ureido)-2-hydroxybenzoic acid;

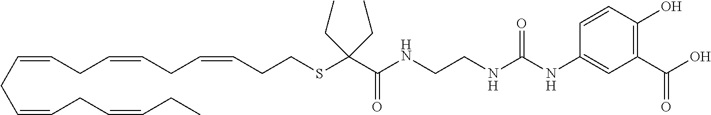 [SMILES: CC/C=C\C/C=C\C/C=C\C/C=C\C/C=C\CCSC(CC)(CC)C(=O)NCCNC(=O)Nc1ccc(O)c(C(=O)O)c1]

5-(3-(2-(2-ethyl-2-((3Z,6Z,9Z,12Z,15Z)-octadeca-3,6,9,12,15-pentaen-1-ylthio)butanamido)ethyl)ureido)-2-hydroxybenzoic acid;

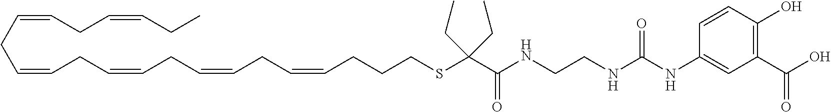 [SMILES: CC/C=C\C/C=C\C/C=C\C/C=C\C/C=C\C/C=C\CCCSC(CC)(CC)C(=O)NCCNC(=O)Nc1ccc(O)c(C(=O)O)c1]

5-(3-(2-(2-((4Z,7Z,10Z,13Z,16Z,19Z)-docosa-4,7,10,13,16,19-hexaen-1-ylthio)-2-ethylbutanamido)ethyl)ureido)-2-hydroxybenzoic acid;

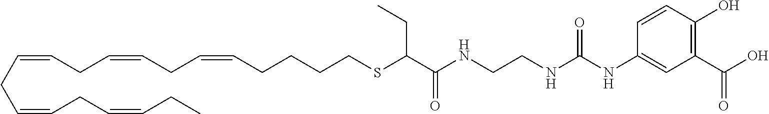 [SMILES: CC/C=C\C/C=C\C/C=C\C/C=C\C/C=C\CCCCSC(CC)C(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-ylthio)butanamido)ethyl)ureido)benzoic acid;

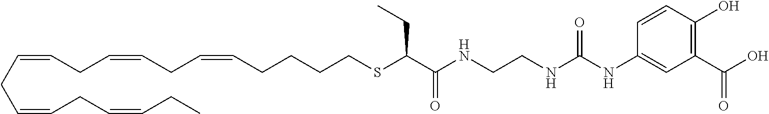 [SMILES: CC/C=C\C/C=C\C/C=C\C/C=C\C/C=C\CCCCS[C@@H](CC)C(=O)NCCNC(=O)Nc1ccc(O)c(C(=O)O)c1]

2-hydroxy-5-(3-(2-((S)-2-((5Z,8Z,11Z,14Z,17Z)-icosa-5,8,11,14,17-pentaen-1-ylthio)butanamido)ethyl)ureido)benzoic acid;

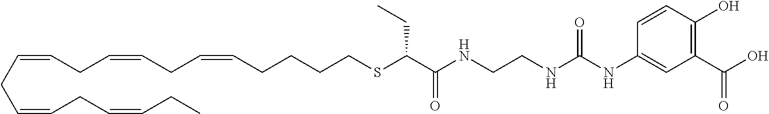 [SMILES: CC/C=C\C/C=C\C/C=C\C/C=C\C/C=C\CCCCS[C@H](CC)C(=O)NCCNC(=O)Nc1ccc(O)c(C(=O)O)c1]

2-hydroxy-5-(3-(2-((R)-2-((5Z,8Z,11Z,14Z,17Z)-icosa-5,8,11,14,17-pentaen-1-ylthio)butanamido)ethyl)ureido)benzoic acid;

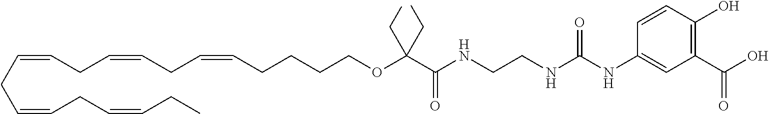 [SMILES: CC/C=C\C/C=C\C/C=C\C/C=C\C/C=C\CCCCOC(CC)(CC)C(=O)NCCNC(=O)Nc1ccc(O)c(C(=O)O)c1]

5-(3-(2-(2-ethyl-2-((5Z,8Z,11Z,14Z,17Z)-icosa-5,8,11,14,17-pentaen-1-yloxy)butanamido)ethyl)ureido)-2-hydroxybenzoic acid;

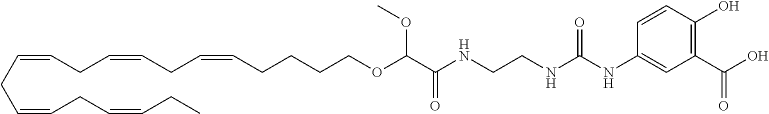 [SMILES: CC/C=C\C/C=C\C/C=C\C/C=C\C/C=C\CCCCOC(OC)C(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-yloxy)-2-methoxyacetamido)ethyl)ureido)benzoic acid;

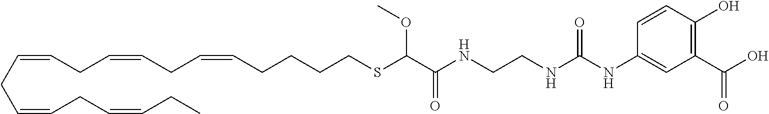 [SMILES: CC/C=C\C/C=C\C/C=C\C/C=C\C/C=C\CCCCSC(OC)C(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-ylthio)-2-methoxyacetamido)ethyl)ureido)benzoic acid;

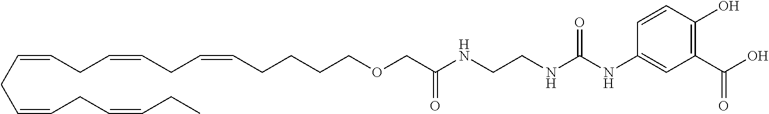 [SMILES: CC/C=C\C/C=C\C/C=C\C/C=C\C/C=C\CCCCOCC(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-yloxy)acetamido)ethyl)ureido)benzoic acid; and

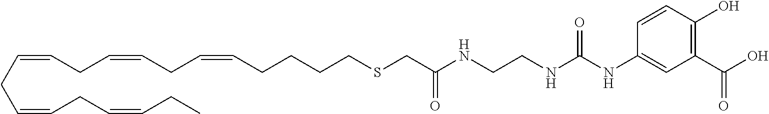 [SMILES: CC/C=C\C/C=C\C/C=C\C/C=C\C/C=C\CCCCSCC(=O)NCCNC(=O)Nc1ccc(O)c(C(=O)O)c1]

2-hydroxy-5-(3-(2-(2-((5Z,8Z,11Z,14Z,17Z)-icosa-5,8,11,14,17-pentaen-1-ylthio)acetamido)ethyl)ureido)benzoic acid.

In at least one embodiment Z is

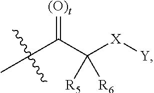

wherein

R₅ is hydrogen and R₆ is an alkyl group, X is CH₂, O, or S, and Y is chosen from a C₁₀-C₂₄ alkenyl having 1-6 double bonds, such as a C₂₀ alkenyl having 5 double bonds, such as a C₂₂ alkenyl having 6 double bonds.

In at least one embodiment Z is

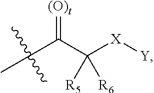

wherein

R₅ is hydrogen and R₆ is an alkyl group, X is CH₂, O, or S, and Y is chosen from a C₁₀-C₂₄ alkenyl having 1-6 double bonds, such as a C₁₋₆ alkenyl having 5 double bonds, such as a C₁₋₉ alkenyl having 6 double bonds.

Exemplary compounds include:

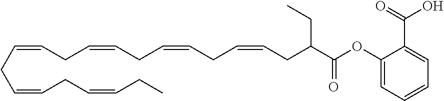 [SMILES: CC/C=C\C/C=C\C/C=C\C/C=C\C/C=C\C/C=C\CC(CC)C(=O)Oc1ccccc1C(=O)O]

2-(((4Z,7Z,10Z,13Z,16Z,19Z)-2-ethyldocosa-4,7,10,13,16,19-hexaenoyl)oxy)benzoic acid;

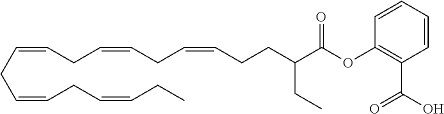 [SMILES: CC/C=C\C/C=C\C/C=C\C/C=C\C/C=C\CCC(CC)C(=O)Oc1ccccc1C(=O)O]

2-(((5Z,8Z,11Z,14Z,17Z)-2-ethylicosa-5,8,11,14,17-pentaenoyl)oxy)benzoic acid;

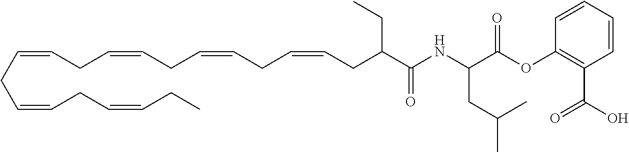 [SMILES: CC/C=C\C/C=C\C/C=C\C/C=C\C/C=C\C/C=C\CC(CC)C(=O)NC(CC(C)C)C(=O)Oc1ccccc1C(=O)O]

2-((2-((4Z,7Z,10Z,13Z,16Z,19Z)-2-ethyldocosa-4,7,10,13,16,19-hexaenamido)-4-methylpentanoyl)oxy)benzoic acid;

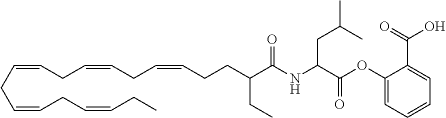 [SMILES: CC/C=C\C/C=C\C/C=C\C/C=C\C/C=C\CCC(CC)C(=O)NC(CC(C)C)C(=O)Oc1ccccc1C(=O)O]

2-((2-((5Z,8Z,11Z,14Z,17Z)-2-ethylicosa-5,8,11,14,17-pentaenamido)-4-methylpentanoyl)oxy)benzoic acid;

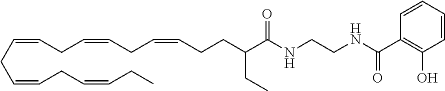 [SMILES: CC/C=C\C/C=C\C/C=C\C/C=C\C/C=C\CCC(CC)C(=O)NCCNC(=O)c1ccccc1O]

N-(2-((5Z,8Z,11Z,14Z,17Z)-2-ethylicosa-5,8,11,14,17-pentaenamido)ethyl)-2-hydroxybenzamide;

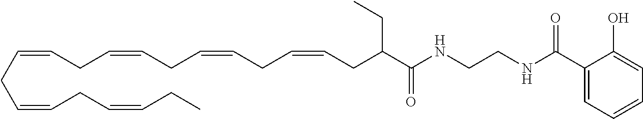 [SMILES: CC/C=C\C/C=C\C/C=C\C/C=C\C/C=C\C/C=C\CC(CC)C(=O)NCCNC(=O)c1ccccc1O]

N-(2-((4Z,7Z,10Z,13Z,16Z,19Z)-2-ethyldocosa-4,7,10,13,16,19-hexaenamido)ethyl)-2-hydroxybenzamide;

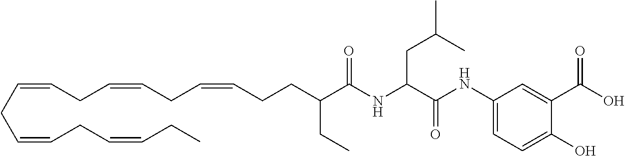 [SMILES: CC/C=C\C/C=C\C/C=C\C/C=C\C/C=C\CCC(CC)C(=O)NC(CC(C)C)C(=O)Nc1ccc(O)c(C(=O)O)c1]

5-(2-((5Z,8Z,11Z,14Z,17Z)-2-ethylicosa-5,8,11,14,17-pentaenamido)-4-methylpentanamido)-2-hydroxybenzoic acid;

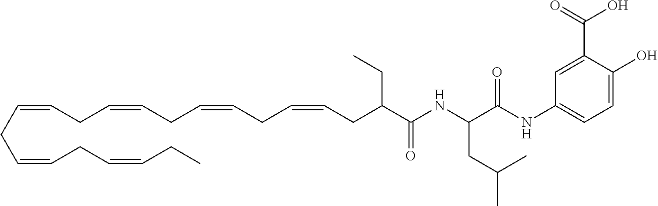 [SMILES: CC/C=C\C/C=C\C/C=C\C/C=C\C/C=C\C/C=C\CC(CC)C(=O)NC(CC(C)C)C(=O)Nc1ccc(O)c(C(=O)O)c1]

5-(2-((4Z,7Z,10Z,13Z,16Z,19Z)-2-ethyldocosa-4,7,10,13,16,19-hexaenamido)-4-methylpentanamido)-2-hydroxybenzoic acid;

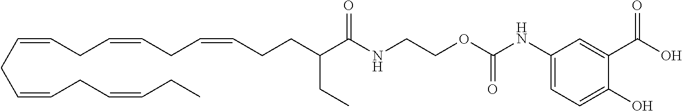 [SMILES: CC/C=C\C/C=C\C/C=C\C/C=C\C/C=C\CCC(CC)C(=O)NCCOC(=O)Nc1ccc(O)c(C(=O)O)c1]

5-(((2-((5Z,8Z,11Z,14Z,17Z)-2-ethylicosa-5,8,11,14,17-pentaenamido)ethoxy)carbonyl)amino)-2-hydroxybenzoic acid;

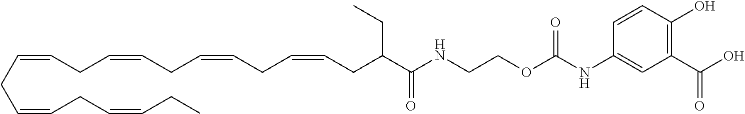 [SMILES: CC/C=C\C/C=C\C/C=C\C/C=C\C/C=C\C/C=C\CC(CC)C(=O)NCCOC(=O)Nc1ccc(O)c(C(=O)O)c1]

5-(((2-((4Z,7Z,10Z,13Z,16Z,19Z)-2-ethyldocosa-4,7,10,13,16,19-hexaenamido)ethoxy)carbonyl)amino)-2-hydroxybenzoic acid;

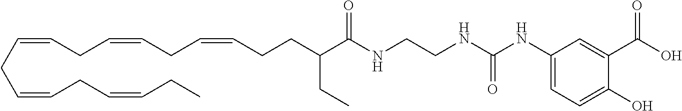 [SMILES: CC/C=C\C/C=C\C/C=C\C/C=C\C/C=C\CCC(CC)C(=O)NCCNC(=O)Nc1ccc(O)c(C(=O)O)c1]

5-(3-(2-((5Z,8Z,11Z,14Z,17Z)-2-ethylicosa-5,8,11,14,17-pentaenamido)ethyl)ureido)-2-hydroxybenzoic acid;

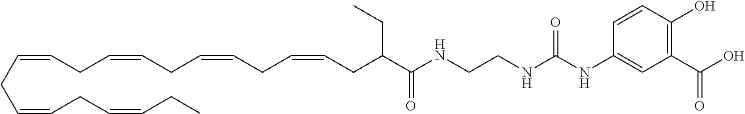 [SMILES: CC/C=C\C/C=C\C/C=C\C/C=C\C/C=C\C/C=C\CC(CC)C(=O)NCCNC(=O)Nc1ccc(O)c(C(=O)O)c1]

5-(3-(2-((4Z,7Z,10Z,13Z,16Z,19Z)-2-ethyldocosa-4,7,10,13,16,19-hexaenamido)ethyl)ureido)-2-hydroxybenzoic acid;

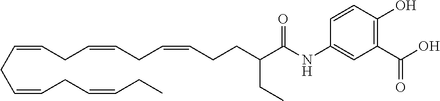 [SMILES: CC/C=C\C/C=C\C/C=C\C/C=C\C/C=C\CCC(CC)C(=O)Nc1ccc(O)c(C(=O)O)c1]

5-((5Z,8Z,11Z,14Z,17Z)-2-ethylicosa-5,8,11,14,17-pentaenamido)-2-hydroxybenzoic acid;

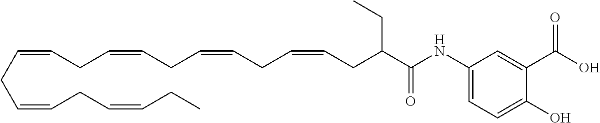 [SMILES: CC/C=C\C/C=C\C/C=C\C/C=C\C/C=C\C/C=C\CC(CC)C(=O)Nc1ccc(O)c(C(=O)O)c1]

5-((4Z,7Z,10Z,13Z,16Z,19Z)-2-ethyldocosa-4,7,10,13,16,19-hexaenamido)-2-hydroxybenzoic acid

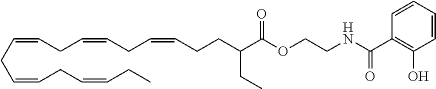 [SMILES: CC/C=C\C/C=C\C/C=C\C/C=C\C/C=C\CCC(CC)C(=O)OCCNC(=O)c1ccccc1O]

(5Z,8Z,11Z,14Z,17Z)-2-(2-hydroxybenzamido)ethyl 2-ethylicosa-5,8,11,14,17-pentaenoate;

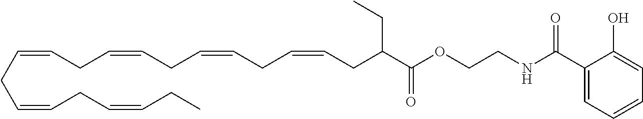 [SMILES: CC/C=C\C/C=C\C/C=C\C/C=C\C/C=C\C/C=C\CC(CC)C(=O)OCCNC(=O)c1ccccc1O]

(4Z,7Z,10Z,13Z,16Z,19Z)-2-(2-hydroxybenzamido)ethyl 2-ethyldocosa-4,7,10,13,16,19-hexaenoate;

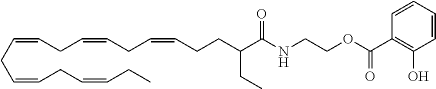 [SMILES: CC/C=C\C/C=C\C/C=C\C/C=C\C/C=C\CCC(CC)C(=O)NCCOC(=O)c1ccccc1O]

2-((5Z,8Z,11Z,14Z,17Z)-2-ethylicosa-5,8,11,14,17-pentaenamido)ethyl 2-hydroxybenzoate; and

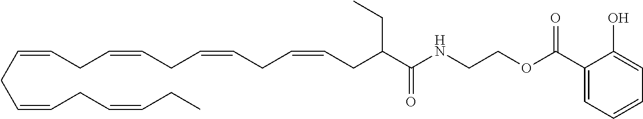 [SMILES: CC/C=C\C/C=C\C/C=C\C/C=C\C/C=C\C/C=C\CC(CC)C(=O)NCCOC(=O)c1ccccc1O]

2-((4Z,7Z,10Z,13Z,16Z,19Z)-2-ethyldocosa-4,7,10,13,16,19-hexaenamido)ethyl 2-hydroxybenzoate.

In at least one embodiment Z is

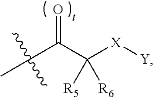

wherein

R₅ and R₆ are hydrogen, X is CH₂, O, or S, and Y is chosen from a C₁₀-C₂₄ alkenyl having 1-6 double bonds, such as a C₁₋₄ alkenyl having 1 double bond, or a C₁₀-C₂₂ alkynyl having 1 triple bond, such as C₁₋₄ alkynyl having 1 triple bond.

Exemplary compounds include:

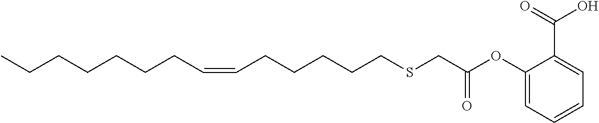 [SMILES: CCCCCCC/C=C\CCCCCSCC(=O)Oc1ccccc1C(=O)O]

(Z)-2-(2-(tetradec-6-en-1-ylthio)acetoxy)benzoic acid;

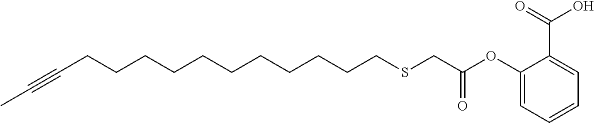 [SMILES: CC#CCCCCCCCCCCCSCC(=O)Oc1ccccc1C(=O)O]

2-(2-(tetradec-12-yn-1-ylthio)acetoxy)benzoic acid;

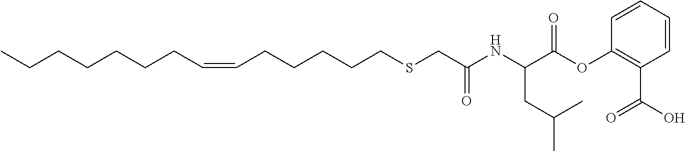 [SMILES: CCCCCCC/C=C\CCCCCSCC(=O)NC(CC(C)C)C(=O)Oc1ccccc1C(=O)O]

(Z)-2-((4-methyl-2-(2-(tetradec-6-en-1-ylthio)acetamido)pentanoyl)oxy)benzoic acid;

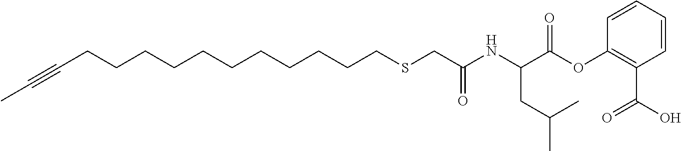 [SMILES: CC#CCCCCCCCCCCCSCC(=O)NC(CC(C)C)C(=O)Oc1ccccc1C(=O)O]

2-((4-methyl-2-(2-(tetradec-12-yn-1-ylthio)acetamido)pentanoyl)oxy)benzoic acid;

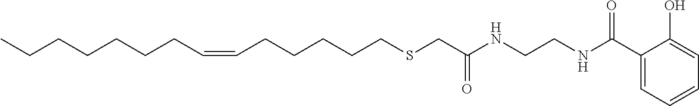 [SMILES: CCCCCCC/C=C\CCCCCSCC(=O)NCCNC(=O)c1ccccc1O]

(Z)-2-hydroxy-N-(2-(2-(tetradec-6-en-1-ylthio)acetamido)ethyl)benzamide;

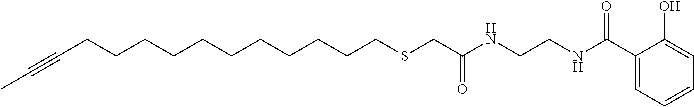 [SMILES: CC#CCCCCCCCCCCCSCC(=O)NCCNC(=O)c1ccccc1O]

2-hydroxy-N-(2-(2-(tetradec-12-yn-1-ylthio)acetamido)ethyl)benzamide;

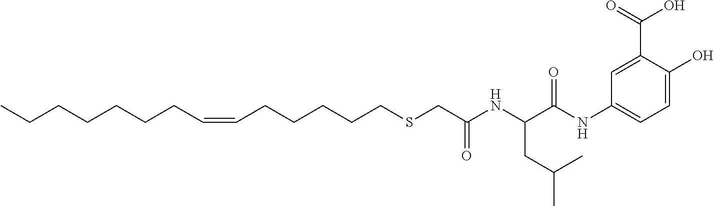 [SMILES: CCCCCCC/C=C\CCCCCSCC(=O)NC(CC(C)C)C(=O)Nc1ccc(O)c(C(=O)O)c1]

(Z)-2-hydroxy-5-(4-methyl-2-(2-(tetradec-6-en-1-ylthio)acetamido)pentanamido)benzoic acid;

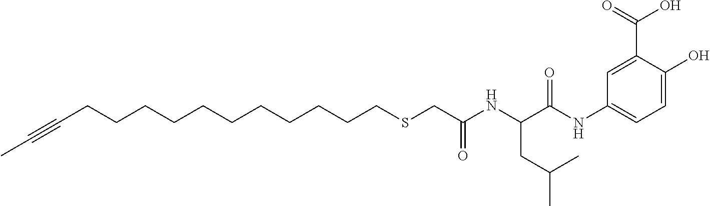 [SMILES: CC#CCCCCCCCCCCCSCC(=O)NC(CC(C)C)C(=O)Nc1ccc(O)c(C(=O)O)c1]

2-hydroxy-5-(4-methyl-2-(2-(tetradec-12-yn-1-ylthio)acetamido)pentanamido)benzoic acid;

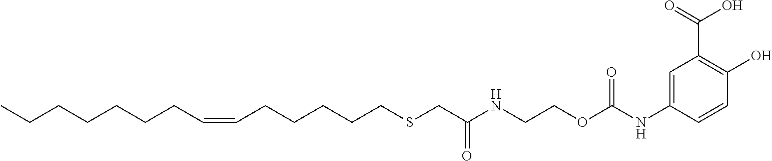 [SMILES: CCCCCCC/C=C\CCCCCSCC(=O)NCCOC(=O)Nc1ccc(O)c(C(=O)O)c1]

(Z)-2-hydroxy-5-(((2-(2-(tetradec-6-en-1-ylthio)acetamido)ethoxy)carbonyl)amino)benzoic acid;

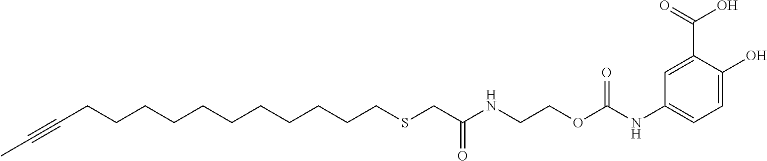 [SMILES: CC#CCCCCCCCCCCCSCC(=O)NCCOC(=O)Nc1ccc(O)c(C(=O)O)c1]

2-hydroxy-5-(((2-(2-(tetradec-12-yn-1-ylthio)acetamido)ethoxy)carbonyl)amino)benzoic acid;

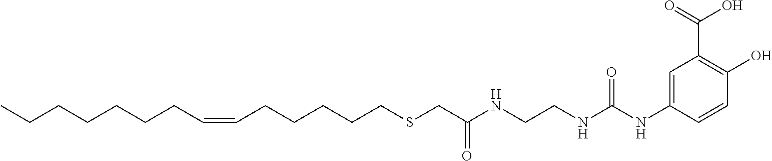 [SMILES: CCCCCCC/C=C\CCCCCSCC(=O)NCCNC(=O)Nc1ccc(O)c(C(=O)O)c1]

(Z)-2-hydroxy-5-(3-(2-(2-(tetradec-6-en-1-ylthio)acetamido)ethyl)ureido)benzoic acid;

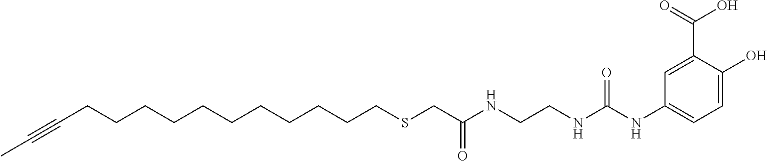 [SMILES: CC#CCCCCCCCCCCCSCC(=O)NCCNC(=O)Nc1ccc(O)c(C(=O)O)c1]

2-hydroxy-5-(3-(2-(2-(tetradec-12-yn-1-ylthio)acetamido)ethyl)ureido)benzoic acid;

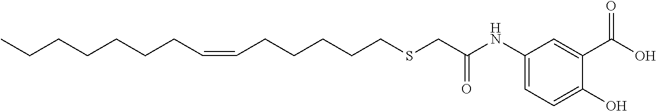 [SMILES: CCCCCCC/C=C\CCCCCSCC(=O)Nc1ccc(O)c(C(=O)O)c1]

(Z)-2-hydroxy-5-(2-(tetradec-6-en-1-ylthio)acetamido)benzoic acid;

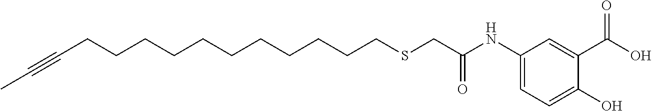 [SMILES: CC#CCCCCCCCCCCCSCC(=O)Nc1ccc(O)c(C(=O)O)c1]

2-hydroxy-5-(2-(tetradec-12-yn-1-ylthio)acetamido)benzoic acid;

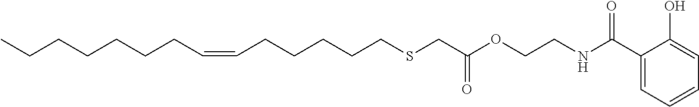 [SMILES: CCCCCCC/C=C\CCCCCSCC(=O)OCCNC(=O)c1ccccc1O]

(Z)-2-(2-hydroxybenzamido)ethyl 2-(tetradec-6-en-1-ylthio)acetate;

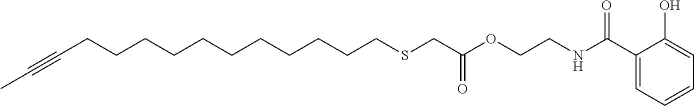 [SMILES: CC#CCCCCCCCCCCCSCC(=O)OCCNC(=O)c1ccccc1O]

2-(2-hydroxybenzamido)ethyl 2-(tetradec-12-yn-1-ylthio)acetate;

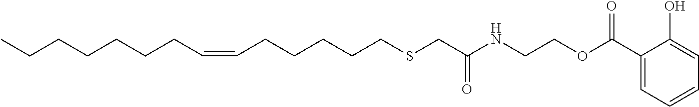 [SMILES: CCCCCCC/C=C\CCCCCSCC(=O)NCCOC(=O)c1ccccc1O]

(Z)-2-(2-(tetradec-6-en-1-ylthio)acetamido)ethyl 2-hydroxybenzoate; and

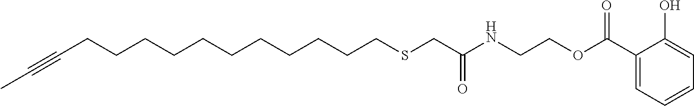 [SMILES: CC#CCCCCCCCCCCCSCC(=O)NCCOC(=O)c1ccccc1O]

2-(2-(tetradec-12-yn-1-ylthio)acetamido)ethyl 2-hydroxybenzoate.

The present disclosure also includes methods for upregulating an anti-inflammatory pathway and downregulating a pro-inflammatory pathway in a cell.

In one embodiment, the method comprises contacting a cell with at least one compound of the present disclosure in an amount sufficient to upregulate an anti-inflammatory pathway and down regulate a pro-inflammatory pathway in the cell. In general, any cell having, or capable of having, inflammatory activity or capable of expressing NFKB can be used. The cell can be provided in any form. For example, the cell can be provided in vitro, ex vivo, or in vivo. Inflammatory activity can be measured using any method known in the art, e.g., methods as disclosed in Tran P. O., et al, Diabetes, 51; 1772-8, 2002. Illustrative examples of cells capable of inflammatory activity include, but are not limited to, immune cells including monocytes, macrophages, T-cell, Th-I, Th-2, Th-17, Treg, lymphocytes, spleen cells, muscle, adipose or fat, vascular cells such as endothelial or pericyte, bone, gum, nerve, brain, glial, astrocytes, nerve, liver, kidney, pancreas including islet cells such as beta cells, lung, heart, breast, bladder, stomach, colon, rectal, small intestine, skin, esophageal, eye, larynx, uterine, ovarian, prostate, tendon, bone marrow, blood, lymph, testicular, vaginal and neoplastic cells.

Also provided in the present disclosure is a method for inhibiting, preventing, or treating inflammation or an inflammatory disease in a subject. The inflammation can be associated with an inflammatory disease or a disease where inflammation contributes to the disease. Inflammatory diseases can arise where there is an inflammation of the body tissue. These include local inflammatory responses and systemic inflammation. Examples of such diseases include, but are not limited to: organ transplant rejection; reoxygenation injury resulting from organ transplantation (see Grupp et al, J. Mol. Cell Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung diseases such as asthma, adult respiratory distress syndrome, chronic obstructive airway disease, and cystic fibrosis; inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis. Metabolic disease such as type 2 diabetes mellitus; the prevention of type 1 diabetes; dyslipedemia; hypertriglyceridemia; diabetic complications, including, but not limited to glaucoma, retinopathy, macula edema, nephropathy, such as microalbuminuria and progressive diabetic nephropathy, polyneuropathy, diabetic neuropathy, atherosclerotic coronary arterial disease, peripheral arterial disease, nonketotic hyperglycemichyperosmolar coma, mononeuropathies, autonomic neuropathy, joint problems, and a skin or mucous membrane complication, such as an infection, a shin spot, a candidal infection or necrobiosis lipoidica diabeticorum; immune-complex vasculitis, systemic lupus erythematosus; inflammatory diseases of the heart such as cardiomyopathy, ischemic heart disease hypercholesterolemia, and atherosclerosis; as well as various other diseases that can have significant inflammatory components, including preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, arrhythmia, prevention of sudden death, muscle wasting diseases such as Duchenne's Muscular Dystrophy, inflammatory myopathies such as dermatomositis, inclusion body myositis, and polymyositis, and cancer cachexia. Also inflammation that results from surgery and trauma can be treated with at least one compound of the present disclosure.

Also provided for by the present disclosure is a method for preventing or treating peripheral insulin resistance comprising administering at least one compound of the present disclosure to a subject in need thereof.

In at least one embodiment is provided for a method of lowering cholesterol, such as non-HDL cholesterol, such as LDL cholesterol and VLDL cholesterol, comprising administering at least one compound of the present disclosure to a subject in need thereof.

In some embodiments, the subject is administered an effective amount of at least one compound of the present disclosure.

The compounds of the present disclosure can each be administered in amounts that are sufficient to treat or prevent an inflammatory disease or a reperfusion disease and/or prevent the development thereof in subjects. The compounds of the present disclosure can each be administered in amounts that are sufficient to treat or prevent any one of the other conditions disclosed herein.

Administration of the compounds of the present disclosure can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Compositions can be prepared according to conventional mixing, granulating or coating methods, respectively, and the present pharmaceutical compositions can contain from about 0.1% to about 99%, from about 5% to about 90%, or from about 1% to about 20% of at least one compound of the present disclosure by weight or volume.

The dosage regimen utilizing the at least one compound of the present disclosure is selected in accordance with a variety of factors including type, species, age, weight, sex and medical condition of the patient; the severity of the condition to be treated; the route of administration; the renal or hepatic function of the patient; and the particular compound of the present disclosure employed. A physician or veterinarian of ordinary skill in the art can readily determine and prescribe the effective amount of the drug required to prevent, counter or arrest the progress of the condition.

Effective dosage amounts of the compounds of the present disclosure, when used for the indicated effects, range from about 20 mg to about 5000 mg of at least one compound of the present disclosure per day. Compositions for in vivo or in vitro use can contain about 20 mg, 50 mg, 75 mg, 100 mg, 150 mg, 250 mg, 500 mg, 750 mg, 1000 mg, 1250 mg, 2500 mg, or 3500 mg of at least one compound of the present disclosure. In a preferred embodiment, the range is from 20 mg to about 1000 mg. In one embodiment, the compositions are in the form of a tablet that can be scored. Effective plasma levels of the compound of the present disclosure can range from about 0.002 mg to about 100 mg per kg of body weight per day. Appropriate dosages of the compounds of the present disclosure can be determined as set forth in L. S. Goodman, et al., The Pharmacological Basis of Therapeutics, 201-26 (5th ed. 1975).

Compounds of the present disclosure can be administered in a single daily dose, or the total daily dosage can be administered in divided doses of two, three or four times daily. Furthermore, compounds of the present disclosure can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of at least one compound of the present disclosure ranges from about 0.1% to about 15%, w/w or w/v.

General Methods for the Synthesis of Compounds Described Herein.

The compounds of general formulas I and II can be prepared by the following general procedures:

Compounds of formula A, E, J, K, P, U, Y and AF are commercially available, or they are known in the literature, or they can be prepared by standard processes known in the art.

Compounds of formula B can be prepared according to method XII, XIII, XIV, XV and XVI.

One of the reactions mentioned herein is the formation of peptide (amide) bonds. These can be formed by combining a carboxylic acid and an amine in the presence of a suitable coupling reagent (e.g., EDC, DCC, CDI or TBTU), optionally in the presence of an amine base (e.g., triethylamine or NMM) and/or a catalyst (e.g., DMAP) in a suitable solvent system. Alternatively, the amine can react with an activated acid halide derivative of the carboxylic acid, for example an acid chloride, in the presence of an amine base (such as those mentioned above) and/or a catalyst (e.g., DMAP) in a suitable solvent system.

One of the reactions mentioned herein is the formation of ester bonds. These can be formed by combining a carboxylic acid and an alcohol in the presence of a suitable coupling reagent (e.g., EDC, DCC, CDI or TBTU), optionally in the presence of an amine base (e.g., triethylamine or NMM) and/or a catalyst (e.g., DMAP) in a suitable solvent system. Alternatively, the alcohol can react with an activated acid halide derivative of the carboxylic acid, for example an acid chloride, in the presence of an amine base (such as those mentioned above) and/or a catalyst (e.g., DMAP) in a suitable solvent system. Ester bonds can also be formed by coupling together a carboxylic acid and an alcohol under classic or non-classic Mitsunobu conditions, familiar to persons skilled in the art.

In some of the reactions mentioned herein it is necessary, or desirable, to protect certain functional groups in order to avoid unwanted side reactions. In the instances where protection is necessary, or desirable, conventional protecting groups may be used. Compounds containing such protecting groups may be commercially available, or they may be prepared by standard processes known in the art from commercially available starting materials (for illustrations see Greene, et al. Protecting Groups in Organic Chemistry, 4^{th} ed. John Wiley & Sons, 2007).

Suitable protecting groups for a hydroxy group include acyl groups (e.g., an alkanoyl group such as acetyl), aroyl groups (e.g., benzoyl) or aryl methyl groups (e.g., benzyl). A suitable protecting group for a carboxyl group is an esterifying group (e.g., a methyl, ethyl, tert-butyl or a benzyl group). Suitable protecting groups for an arylamino or alkylamino group include alkoxycarbonyl groups (e.g., a tert-butoxycarbonyl (t-BOC) group) or arylmethoxycarbonyl groups (e.g., a 9-fluorenylmethyloxycarbonyl (Fmoc) or a carboxybenzyl (Cbz) group).

The deprotection conditions for the above mentioned protecting groups vary with the choice of protecting group and the nature of the compound where they are present. A selection of methods can be found in Greene, et al. Protecting Groups in Organic Chemistry, 4^{th} ed. John Wiley & Sons, 2007. The deprotection of a hydroxy group protected as an acyl group, for example an alkanoyl group or an aroyl group, may be performed by hydrolysis with a suitable base such as an alkali metal hydroxide (e.g., LiOH, NaOH or KOH) in an appropriate solvent system. An arylmethyl group, such as a benzyl group, may be removed, for example, by hydrogenation over a catalyst such as palladium-on-carbon, in an appropriate solvent system. An esterifying group, such as a methyl or an ethyl group, may be cleaved from a carboxy group, for example, by alkaline hydrolysis using a base such as an alkali metal hydroxide, (e.g., LiOH, NaOH or KOH) or by using an organic base (e.g., Et₃N) together with an inorganic salt (e.g., LiCl) in an appropriate solvent system. A tert-butyl group may be removed by treatment, for example, with an acid (e.g., organic acid such as trifluoroacetic acid or formic acid) in an appropriate solvent system. An arylmethyl group, such as a benzyl group, may be removed, for example, by hydrogenation over a catalyst such as palladium-on-carbon, in an appropriate solvent system. The deprotection of an arylamino or alkylamino group, protected as an alkoxycarbonyl group, for example a tort-butoxycarbonyl (t-BOC) group, may be performed by treatment with a suitable acid, such as HCl or trifluoroacetic acid, in an appropriate solvent system. An arylmethoxycarbonyl group, such as a carboxybenzyl (Cbz) group, may be removed by hydrogenation over a catalyst such as palladium-on-carbon, in an appropriate solvent system. 9-Fluorenylmethyloxycarbonyl (Fmoc) may be cleaved, for example, by treatment with a suitable base, such as piperidine or morpholine, in an appropriate solvent system.

The preparation of compounds according to these methods may result in mixtures of stereoisomers. If required, these isomers may be separated, for example, by means of chiral resolving agents and/or by chiral column chromatography, through methods known to the person skilled in the art.

Method I:

The compounds described in category A can be prepared according to the following general procedure:

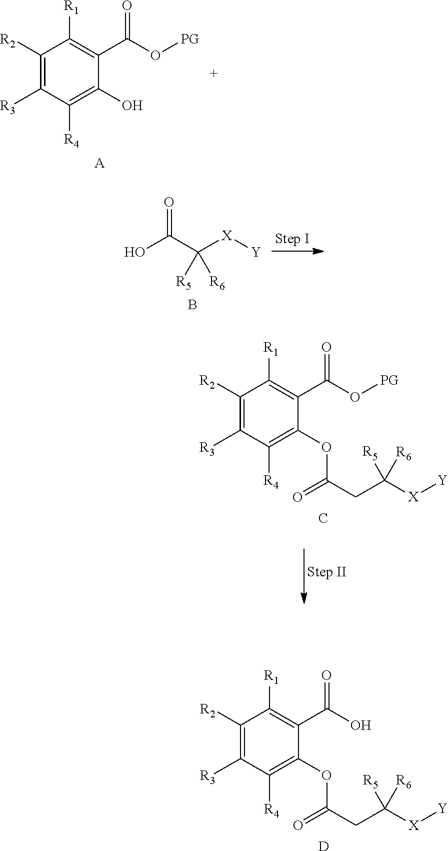

Using method I, compounds of formula A and B can be coupled together forming an ester bond to form compounds of formula C. A suitable protecting group for the carboxyl group present in compounds of formula A may be a methyl group. Cleavage of the protecting group present in compounds of formula C yields compounds of formula D.

Method II:

The compounds described in category B can be prepared according to the following general procedure:

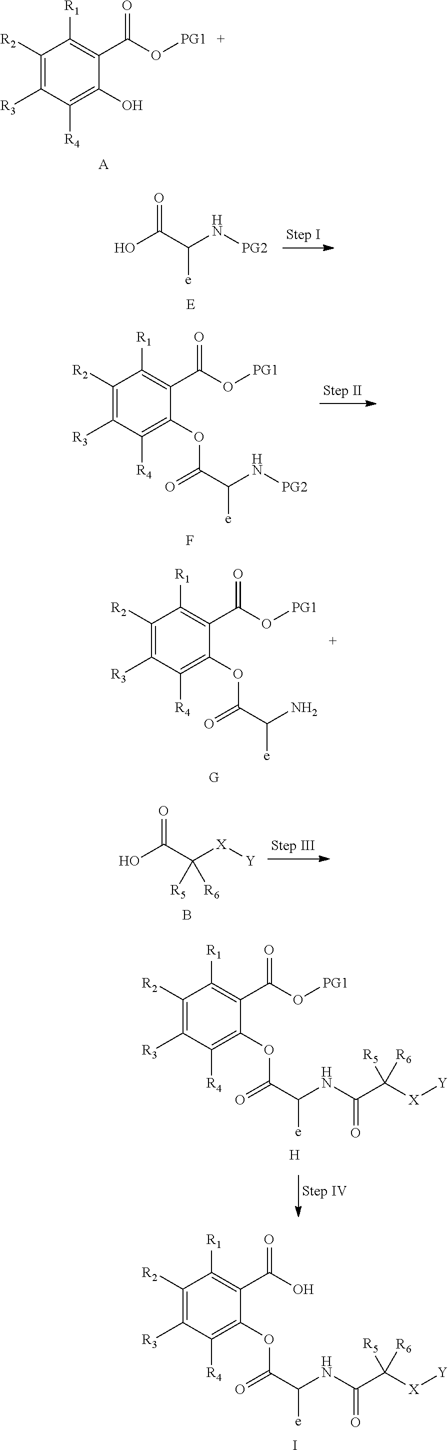

Using method II, compounds of formula A and E can be coupled together forming an ester bond to form compounds of formula F. A suitable protecting group for the carboxyl group present in compounds of formula A (PG1) may be a methyl group. A suitable protecting group for the amino group present in compounds of formula E (PG2) may be a t-BOC group. Removal of PG2 present in compounds of formula F yields compounds of formula G. Compounds of formula G and B can be coupled together forming an amide bond to form compounds of formula H. Cleavage of PG1 present in compounds of formula H yields compounds of formula I.

Alternatively, analogue compounds to those described with the general formula E, where the carboxylic acid is protected with a suitable protecting group (e.g., a methyl group) and where the amine group is unprotected, can react with compounds of formula B forming an amide bond. After cleavage of the protecting group, the obtained compound can be coupled with compounds of formula A forming an ester bond to form compounds of formula H. A suitable protecting group for the carboxyl group present in compounds of formula A may be a methyl group. Cleavage of the protecting group in the obtained product of formula H yields compounds of formula I.

Method III:

The compounds described in category C can be prepared according to the following general procedure:

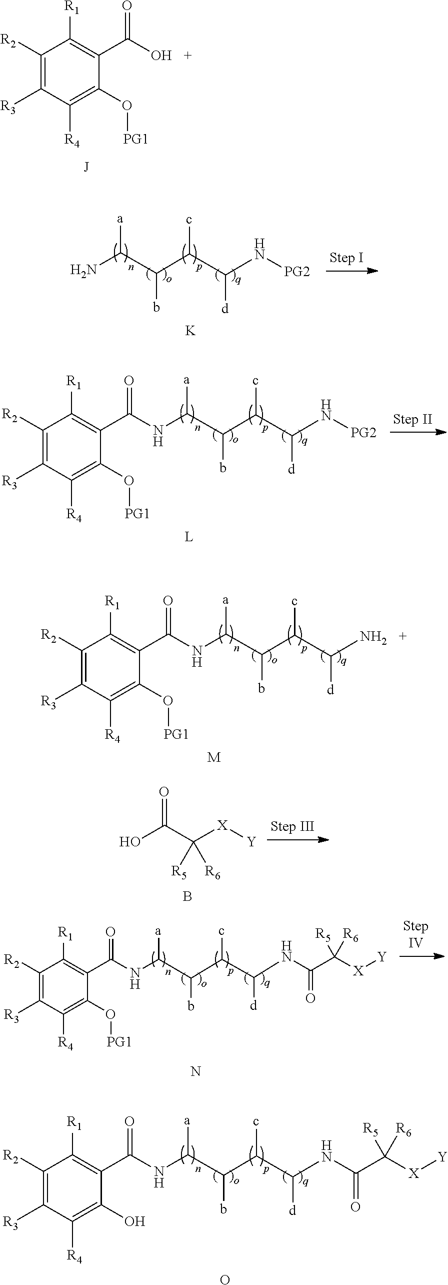

Using method III, compounds of formula J and K can be coupled together forming an amide bond to form compounds of formula L. A suitable protecting group for the hydroxy group present in compounds of formula J (PG1) may be an acetyl group. A suitable protecting group for the amino group present in compounds of formula K (PG2) may be a t-BOC group. Removal of PG2 present in compounds of formula L yields compounds of formula M. Compounds of formula M and B can be coupled together forming an amide bond to form compounds of formula N. Cleavage of PG1 present in compounds of formula N yields compounds of formula O.

Alternatively, compounds of formula K can react with compounds of formula B forming an amide bond. A suitable protecting group for the amino group present in compounds of formula K (PG2) may be a t-BOC group. After cleavage of the protecting group, the obtained amine can be coupled with compounds of formula J forming an amide bond to form compounds of formula N. A suitable protecting group for the carboxyl group present in compounds of formula J may be an acetyl group. Cleavage of the protecting group in the obtained product of formula N yields compounds of formula O.

Method IV:

The compounds described in category D can be prepared according to the following general procedure:

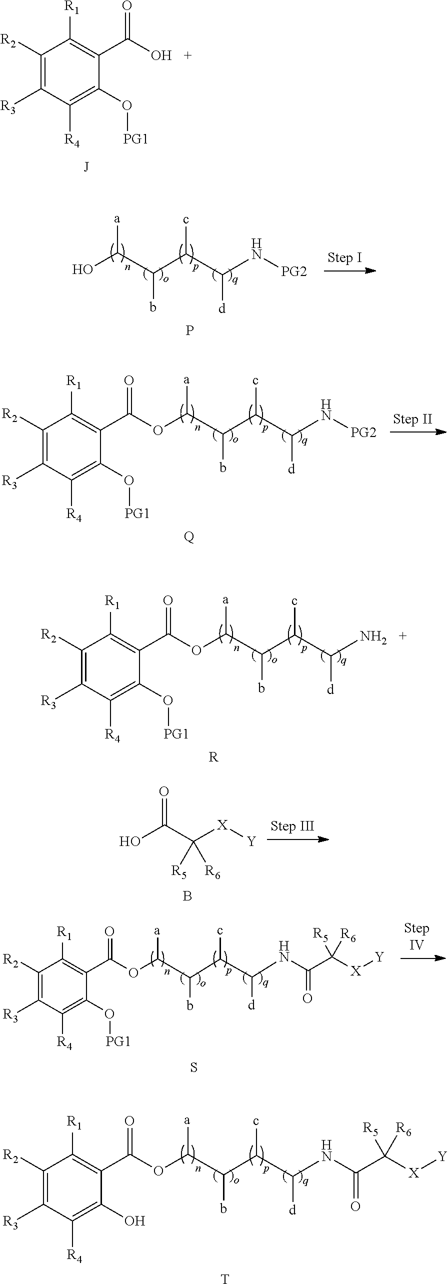

Using method IV, compounds of formula J and P can be coupled together forming an ester bond to form compounds of formula Q. A suitable protecting group for the hydroxyl group, present in compounds of formula J (PG1), may be an acetyl group. A suitable protecting group for the amino group, present in compounds of formula P (PG2), may be a t-BOC group. Removal of PG2, present in compounds of formula Q, yields compounds of formula R. Compounds of formula R and B can be coupled together forming an amide bond to form compounds of formula S. Cleavage of PG1 present in compounds of formula S yields compounds of formula T.

Alternatively, analogue compounds to those described with the general formula P, where the hydroxyl group is protected with a suitable protecting group (e.g., an acetyl group) and where the amine group is unprotected, can react with compounds of formula B forming an amide bond. After cleavage of the protecting group, the obtained compound can be coupled with compounds of formula J forming an ester bond to form compounds of formula S. A suitable protecting group for the carboxyl group present in compounds of formula J may be an acetyl group. Cleavage of the protecting group in the obtained product of formula S yields compounds of formula T.

Method V:

The compounds described in category E can be prepared according to the following general procedure:

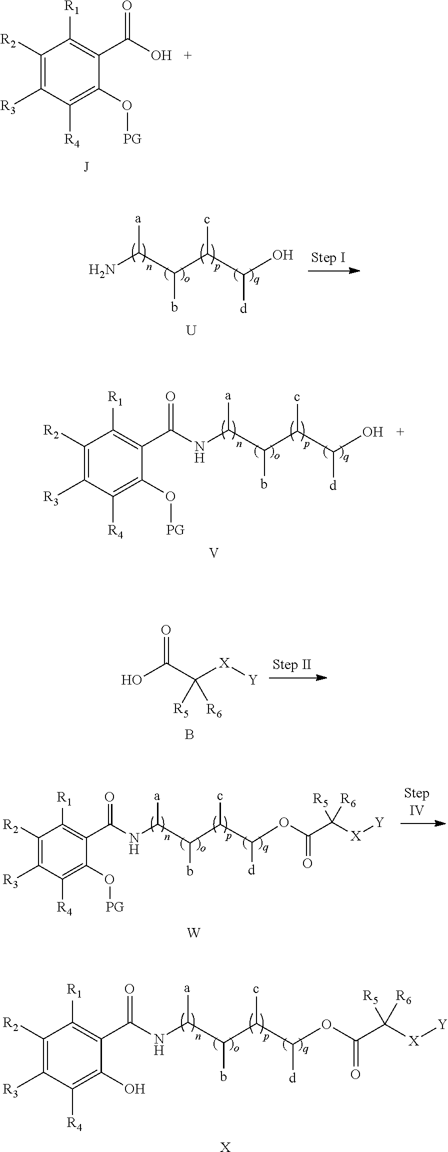

Using method V, compounds of formula J and U can be coupled together forming an amide bond to form compounds of formula V. A suitable protecting group for the hydroxyl group, present in compound J (PG), may be an acetyl group. Compounds of formula V and B can be coupled together forming an ester bond to form compounds of formula W. Cleavage of the protecting group present in compounds of formula W yields compounds of formula X.

Method VI:

The compounds described in category F can be prepared according to the following general procedure:

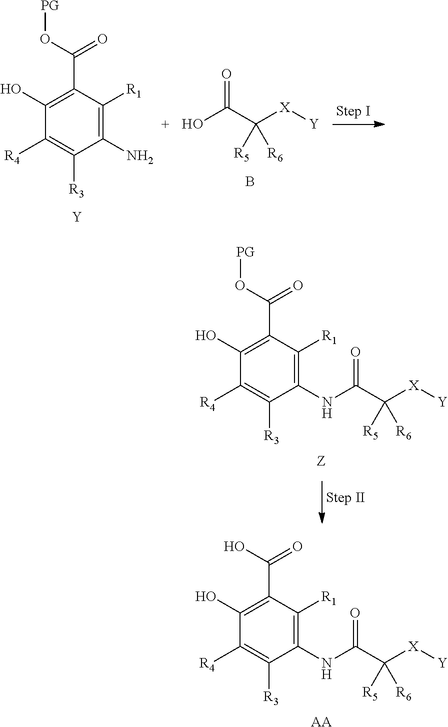

Using method V, compounds of formula Y and B can be coupled together forming an amide bond to form compounds of formula Z. A suitable protecting group for the carboxyl group present in compounds of formula Y may be a methyl group. Cleavage of the protecting group present in compounds of formula Z yields compounds of formula AA.

Method VII:

The compounds described in category G can be prepared according to the following general procedure:

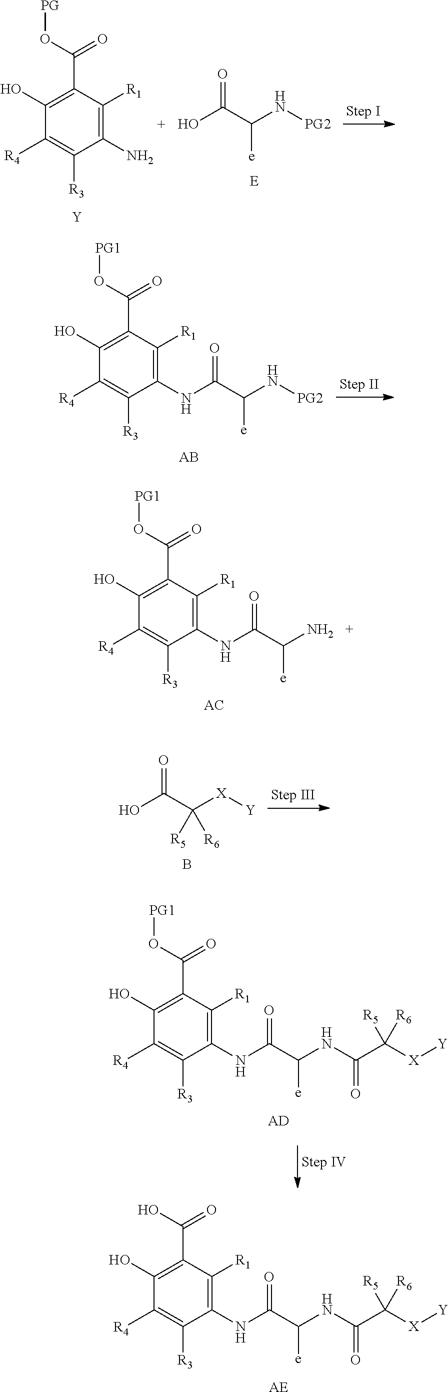

Using method VII, compounds of formula Y and E can be coupled together forming an amide bond to form compounds of formula AB. A suitable protecting group for the carboxyl group present in compounds of formula Y (PG1) may be a methyl group. A suitable protecting group for the amino group present in compounds of formula E (PG2) may be a t-BOC group. Removal of PG2 present in compounds of formula AB yields compounds of formula AC. Compounds of formula AC and B can be coupled together forming an amide bond to form compounds of formula AD. Cleavage of PG1 present in compounds of formula AD yields compounds of formula AE.

Alternatively, analogue compounds to those described with the general formula E, where the carboxylic acid is protected with a suitable protecting group (e.g., a methyl group) and where the amine group is unprotected, can react with compounds of formula B forming an amide bond. After cleavage of the protecting group, the obtained compound can be coupled with compounds of formula Y forming an amide bond to form compounds of formula AD. A suitable protecting group for the carboxyl group present in compounds of formula Y may be a methyl group. Hydrolysis of the protecting group in the obtained product of formula AD yields compounds of formula AE.

Method VIII:

The compounds described in category H can be prepared according to the following general procedure:

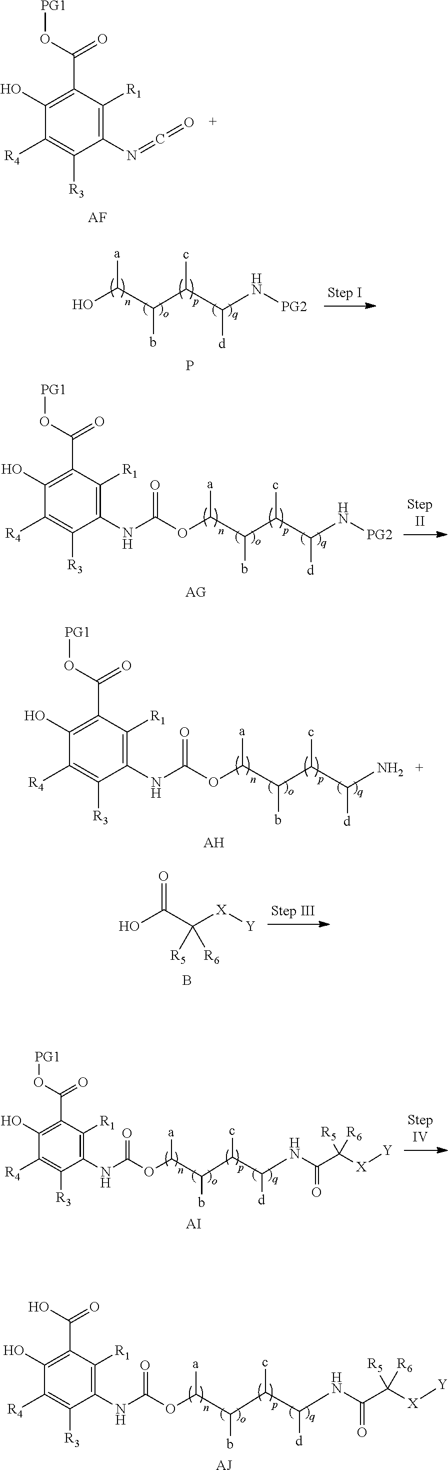

Using method VIII, addition reactions between compounds of formula AF and P, under conditions known to the person skilled in the art, give compounds of formula AG. A suitable protecting group for the carboxyl group present in compounds of formula AF (PG1) may be a methyl group. A suitable protecting group for the amino group present in compounds of formula P (PG2) may be a t-BOC group. Removal of PG2 present in compounds of formula AG yields compounds of formula AH. Compounds of formula AH and B can be coupled together forming an amide bond to form compounds of formula AI. Cleavage of PG1 present in compounds of formula AI yields compounds of formula AJ.

Alternatively, analogue compounds to those described with the general formula P, where the hydroxyl group is protected with a suitable protecting group, for example an acetyl group, and where the amine group is unprotected, can react with compounds of formula B forming an amide bond. After cleavage of the protecting group, the obtained alcohol can be added to compounds of formula AF, under conditions known to the skilled person in the art, to form compounds of formula AI. A suitable protecting group for the carboxyl group present in compounds of formula AF may be a methyl group. Cleavage of the protecting group in the obtained product of formula AI yields compounds of formula AJ.

Method IX:

The compounds described in category H can be prepared according to the following general procedure:

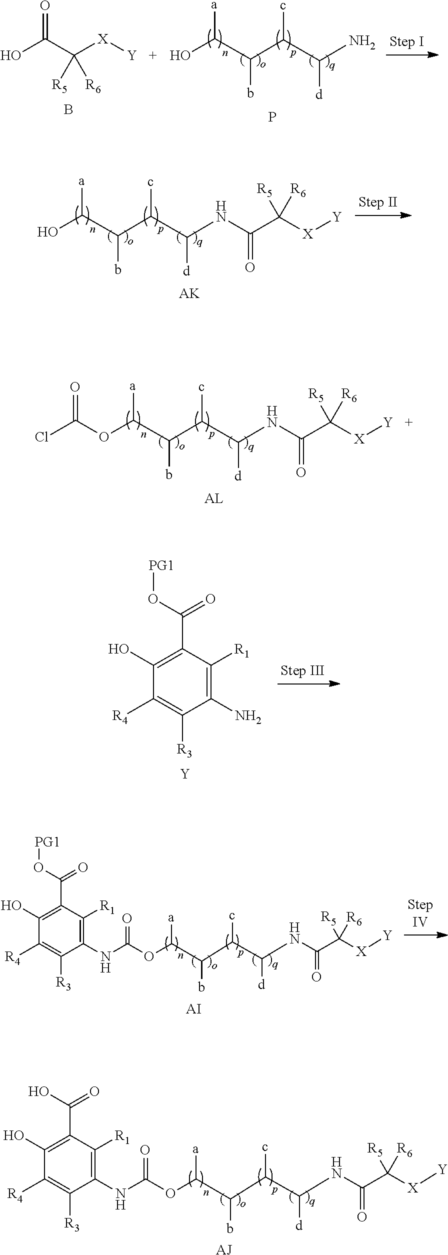

Using method IX, compounds of formula B and P can be coupled together forming an amide bond to form compounds of formula AK. Compounds of formula AK can be converted into compounds of formula AL, for example, by means of a nucleophilic acyl substitution between AK and for example trichloromethyl chloroformate, in the presence of a suitable base, for example an amine base (e.g., N,N-diisopropylethylamine) in a suitable solvent system. A nucleophilic acyl substitution between AL and Y under similar conditions as described above, gives compounds of formula AI. A suitable protecting group for the carboxyl group present in compounds of formula Y (PG1) may be a methyl group. Cleavage of PG1 present in compounds of formula AI yields compounds of formula AJ.

Method X:

The compounds described in category I can be prepared according to the following general procedure:

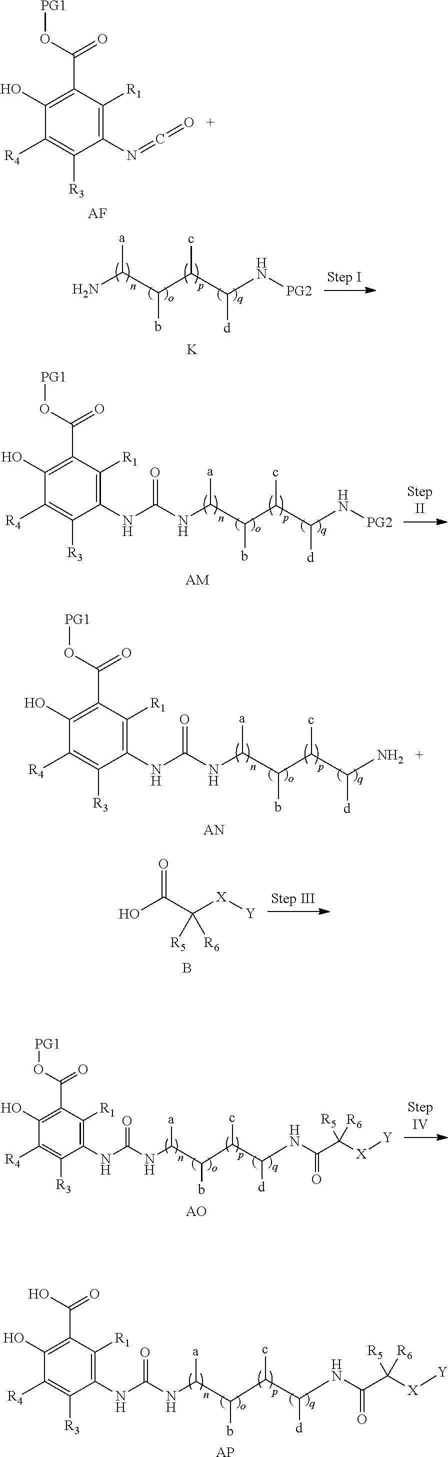

Using method X, addition reactions between compounds of formula AF and K, under conditions known to the person skilled in the art, give compounds of formula AM. A suitable protecting group for the carboxyl group, present in compounds of formula AF (PG1), may be a methyl group. A suitable protecting group for the amino group present in compounds of formula K (PG2) may be a t-BOC group. Removal of PG2 present in compounds of formula AM yields compounds of formula AN. Compounds of formula AN and B can be coupled together forming an amide bond to form compounds of formula AO. Removal of PG1 present in compounds of formula AO yields compounds of formula AP.

Alternatively, compounds of formula K can react with compounds of formula B forming an amide bond. A suitable protecting group for the amino group, present in compounds of formula K (PG2), may be a t-BOC group. After cleavage of the protecting group, the obtained amine can be added to compounds of formula AF, under conditions known to the skilled person in the art, to form compounds of formula AO. A suitable protecting group for the carboxyl group, present in compounds of formula AF, may be a methyl group. Cleavage of the protecting group in compounds of formula AO yields compounds of formula AP.

Method XI:

The compounds described in category I can be prepared according to the following general procedure:

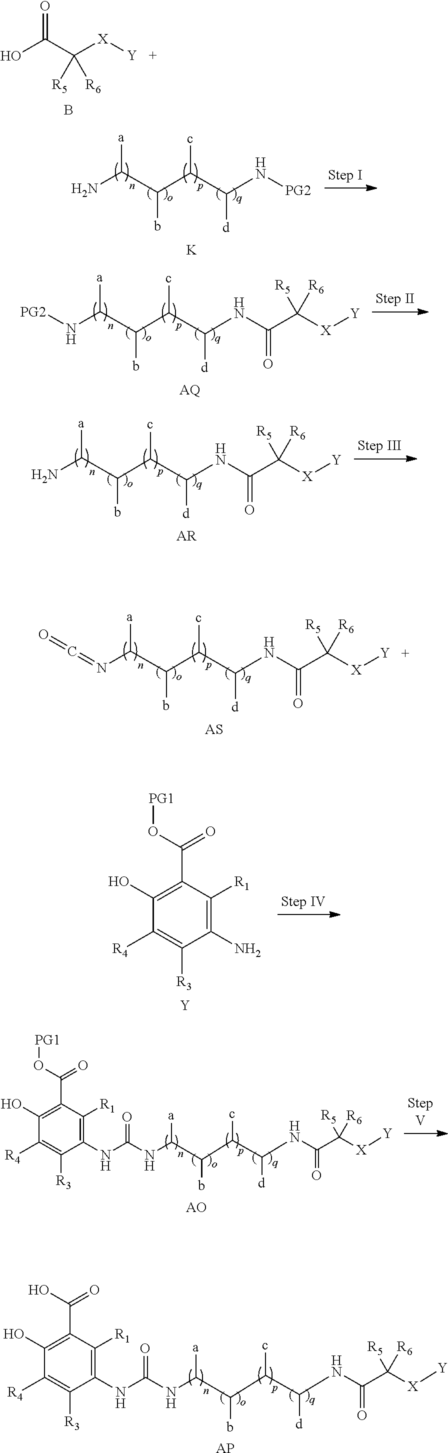

Using method XI, compounds of formula B and K can be coupled together forming an amide bond to form compounds of formula AQ. A suitable protecting group for the amino group present in compounds of formula K (PG2) may be a t-BOC group. Removal of PG2 present in compounds of formula AQ yields compounds of formula AR. Compounds of formula AR can be converted, using functional group interconversion, into isocyanates (compounds of formula AS), by for example reacting them with trichloromethyl chloroformate, in the presence of a suitable base, for example an amine base (e.g., 1,8-bis(dimethylamino)-naphthalene) in a suitable solvent system. An addition reaction between compounds of formula AS and Y, under conditions known to the person skilled in the art, gives compounds of formula AO. A suitable protecting group for the carboxyl group present in compounds of formula Y (PG1) may be a methyl group. Removal of PG1 present in compounds of formula AO yields compounds of formula AP.

The compounds of general formula B can be prepared by the following general procedures:

Compounds of formula AT, AU, AX and AY are commercially available, or they are known in the literature, or they are prepared by standard processes known in the art.

The A-group represents a carboxylic acid or a derivative thereof, such as a carboxylic ester. If the acid derivatives used are carboxylic esters, hydrolysis can be performed to obtain the free fatty acids.

The leaving group (LG) present in compounds of formula AU and AW, may, for example, be mesylate, tosylate or a suitable halogen (e.g., bromine or iodine).

Methods XII, XIII and XIV can be applied in order to obtain compounds of formula B where X is O (named AV).

Methods XV and XVI can be applied in order to obtain compounds of formula B where X is S (named AZ) or, SO or SO₂ (named AAA).

Method XII:

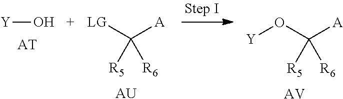

Using method XII, alcohols of formula AT can react in a substitution reaction with compounds of formula AU, in the presence of base, such as an alkali metal hydroxide (e.g., NaOH) in an appropriate solvent system, to form compounds of formula AV. Appropriate solvent systems include two phase mixtures such as toluene and water, which may require the use of a phase transfer catalyst, such as a quaternary ammonium salts (e.g., tetrabutylammonium chloride).

Method XIII:

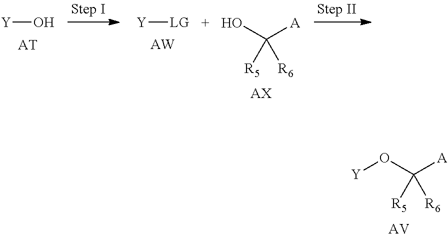

Using method XIII, alcohols of formula AT can be converted using functional group interconversion, by methods familiar to persons skilled in the art, into compounds where the terminal hydroxy group have been transformed into a suitable leaving group (LG). A bromide, for example can be formed by treating the alcohols with carbon tetrabromide and triphenylphosphine in an appropriate solvent system. These compounds can be reacted further (step II), in a substitution reaction with an appropriately substituted hydroxy acetic acid derivative (compounds of formula AX), in the presence of base, such as an alkali metal hydroxide (e.g., NaOH) in an appropriate solvent system, to form compounds of formula AV. Appropriate solvent systems include two phase mixtures such as toluene and water, which may require the use of a phase transfer catalyst, such as a quaternary ammonium salts (e.g., tetrabutylammonium chloride).

Method XIV:

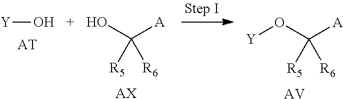

Using method XIV, alcohols of formula AT can react with the appropriately substituted hydroxy acetic acid derivatives (compounds of formula AX), under classic or non-classic Mitsunobu conditions, using methods familiar to persons skilled in the art, to yield compounds of formula AV.

Method XV:

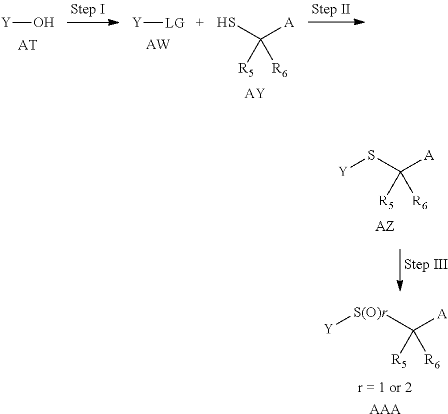

Using method XV, alcohols of formula AT can be converted, using functional group interconversion, by methods familiar to persons skilled in the art (step I), into compounds where the terminal hydroxy group have been transformed into a suitable leaving group (LG). These compounds can be reacted further (step II), in a substitution reaction with the appropriately substituted thiol acetic acid derivatives (compounds of formula AY), in the presence of base (e.g., sodium ethoxide), in a suitable solvent system to yield compounds of formula AZ.

The corresponding sulfoxides and sulfones can be prepared by oxidation of the thioethers (compounds of formula AZ) with a suitable oxidising agent (step III). Examples of oxidising agents are m-chloro-perbenzoic acid (MCPBA), hydrogen peroxide (H₂O₂) and oxone (potassium peroxymonosulfate). By using 1 equivalent or less of the oxidising agent, the main product will be the sulfoxide. By using an excess of the oxidising agent (e.g., 2 equivalents) the main product will be the sulfone.

Method XVI:

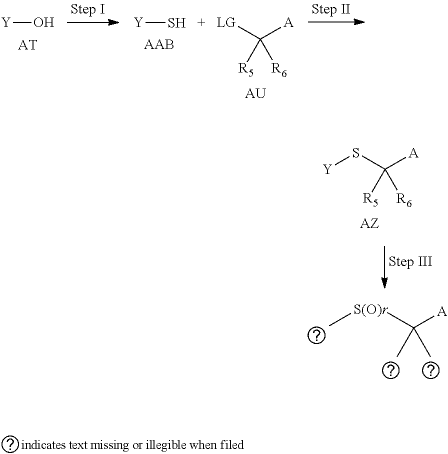

Using method XVI, alcohols of formula AT can be converted to the corresponding thiols (compounds of formula AAB). These can be formed, for example, by first reacting the alcohols with ethanethioic S-acid, for example under Mitsunobu conditions, to form thioesters. The thioesters can then be converted to the thiols by for example hydrolysis, by means of a suitable base (e.g., K₂CO₃ or an alkali metal hydroxide such as LiOH, NaOH or KOH), or by reduction, by means of a reducing agent (e.g., LiAlH₄), in an appropriate solvent system. The thiols can then be reacted further (step II), in a substitution reaction with compounds of formula AU, in the presence of base (such as sodium ethoxide), in an appropriate solvent system to give compounds of formula AZ.

The corresponding sulfoxides and sulfones can be prepared by oxidation of the thioethers (compounds of formula AZ) with a suitable oxidising agent (step III) as described in method XV.

### EXAMPLES

The present disclosure may be further described by the following non-limiting examples, in which standard techniques known to the skilled chemist and techniques analogous to those described in these examples may be used where appropriate. It is understood that the skilled artisan will envision additional embodiments consistent with the disclosure provided herein.

Unless otherwise stated:

All reactions were carried out at room temperature (RT), typically in the range between 18-25° C., with solvents of HPLC grade, under anhydrous conditions and stirred using a stirring bar.

All reactions and handling and storage of the compounds described herein, were performed under inert atmosphere where possible.

Evaporations were carried out by rotary evaporation in vacuo.

Column chromatography was performed by the flash procedure on silica gel, or by preparative High-performance Liquid Chromatography (HPLC) on a C18 column, using either a La Prep HPLC with spectral photometer or an Agilent, LC system 1100 series with a diode array detector.

Nuclear magnetic resonance (NMR) shift values were recorded on a Bruker Avance DPX 200 or 300 instrument with peak multiplicities described as follows: s, singlet; d, doublet; dd, double doublet; t, triplet; q, quartet; p, pentet; m, multiplett; br, broad.

Mass spectra were recorded with a G1956A mass spectrometer or a Waters Qtof II mass spectrometer, both with electrospray ionization (ESI).

HR-MS (ESI) was run on a Micromas Q-TOF-2.

Reported yields are illustrative and do not necessarily represent the maximum yield attainable.

### Example 1

### Preparation of tert-butyl 2-((5Z,8Z,11Z,14Z,17Z)-icosa-5,8,11,14,17-pentaen-1-yloxy)butanoate

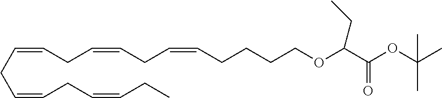 [SMILES: CC/C=C\C/C=C\C/C=C\C/C=C\C/C=C\CCCCOC(CC)C(=O)OC(C)(C)C]

Tetrabutylammonium chloride (0.55 g, 1.98 mmol) was added to a solution of (5Z,8Z,11Z,14Z,17Z)-icosa-5,8,11,14,17-pentaen-1-ol, (3.50 g, 12.1 mmol) in toluene (35 mL). An aqueous solution of NaOH (50% (w/w), 11.7 mL) was added under vigorous stirring at room temperature, followed by t-butyl 2-bromobutyrate (5.41 g, 24.3 mmol). The resulting mixture was heated to 50° C. and additional t-butyl 2-bromobutyrate was added after 1.5 hours (2.70 g, 12.1 mmol), 3.5 hours (2.70 g, 12.1 mmol) and 4.5 hours (2.70 g, 12.1 mmol) and stirred for 12 hours in total. After cooling to room temperature, ice water (25 mL) was added and the resulting two phases were separated. The organic phase was washed with 5% NaOH (aq) and brine, dried (MgSO₄), filtered and concentrated. The residue was purified by flash chromatography using a gradient of 0-5% EtOAc in heptane as eluent. Concentration of the appropriate fractions afforded 1.87 g (36% yield) of the title compound. ¹H NMR (300 MHz, CDCl₃): δ 0.85-1.10 (m, 6H), 1.35-1.54 (m, 11H), 1.53-1.87 (m, 4H), 1.96-2.26 (m, 4H), 2.70-3.02 (m, 8H), 3.31 (dt, 1H), 3.51-3.67 (m, 2H), 5.10-5.58 (m, 10H).

### Example 2

### Preparation of 2-((5Z,8Z,11Z,14Z,17Z)-icosa-5,8,11,14,17-pentaen-1-yloxy)butanoic acid

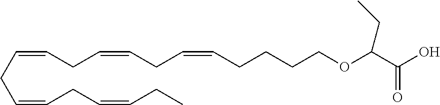 [SMILES: CC/C=C\C/C=C\C/C=C\C/C=C\C/C=C\CCCCOC(CC)C(=O)O]

tert-Butyl 2-((5Z,8Z,11Z,14Z,17Z)-icosa-5,8,11,14,17-pentaen-1-yloxy)butanoate (19.6 g, 45.5 mmol) was dissolved in dichloromethane (DCM) (200 mL) and placed under nitrogen. Trifluoroacetic acid (TFA) (50 mL) was added and the reaction mixture was stirred for one hour. Water was added and the aqueous phase was extracted twice with DCM. The combined organic extract was washed with brine, dried (Na₂SO₄), filtered and concentrated. The residue was subjected to flash chromatography using a gradient of 10-20% EtOAc (containing 1% formic acid (FA) in heptane (also containing 1% FA) as eluent. Concentration of the appropriate fractions afforded 12.1 g (71% yield) of the title compound. ¹H-NMR (300 MHz, CDCl₃): δ 0.90-1.00 (m, 6H), 1.50 (m, 2H), 1.70 (m, 2H), 1.80 (m, 2H), 2.10 (m, 4H), 2.80-2.90 (m, 8H), 3.50 (m, 1H), 3.60 (m, 1H), 3.75 (t, 1H), 5.30-5.50 (m, 10H). MS (ESI): 373.2 [M−H]⁻.

### Example 3

### Preparation of tert-butyl 2-((2-((5Z,8Z,11Z,14Z,17Z)-icosa-5,8,11,14,17-pentaen-1-yloxy)butanoyl)oxy)benzoate

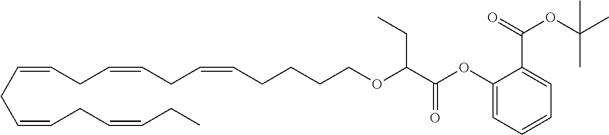 [SMILES: CC/C=C\C/C=C\C/C=C\C/C=C\C/C=C\CCCCOC(CC)C(=O)Oc1ccccc1C(=O)OC(C)(C)C]

1-Ethyl-3-(3-dimethylaminopropyl) carbodiimide (EDC) (316 mg, 1.65 mmol) and 4-dimethylaminopyridine (DMAP) (20 mg, 0.15 mmol) were added to a solution of 2-((5Z,8Z,11Z,14Z,17Z)-icosa-5,8,11,14,17-pentaen-1-yloxy)butanoic acid (561 mg, 1.5 mmol) in DCM (10 mL) and the reaction mixture was stirred for 10 minutes. tert-Butyl 2-hydroxybenzoate (291 mg, 1.5 mmol) was added and the mixture was stirred for 3 hours. Brine was added and the resulting two phases were separated. The aqueous phase was extracted with DCM, and the combined organic phases were dried (Na₂SO₄), filtered and concentrated in vacuo. The residue was purified by flash chromatography using a mixture of 5% EtOAc in heptane as eluent. Concentration of the appropriate fractions afforded 500 mg (61% yield) of the title compound. ¹H NMR (300 MHz, CDCl₃): δ 0.98 (t, 3H), 1.11 (t, 3H), 1.13 (t, 3H), 1.45-1.75 (m, 4H), 1.56 (s, 9H), 1.90-2.20 (m, 6H), 2.80-2.90 (m, 8H), 3.45-3.60 (m, 1H), 3.80-3.90 (m, 1H), 4.05-4.15 (m, 1H), 5.25-5.50 (m, 10H), 7.06 (m, 1H), 7.28 (m, 1H), 7.52 (m, 1H), 7.89 (m, 1H). MS (ESI): 573 [M+Na]⁺.

### Example 4

### Preparation of 2-((2-((5Z,8Z,11Z,14Z,17Z)-icosa-5,8,11,14,17-pentaen-1-yloxy)butanoyl)oxy)benzoic acid

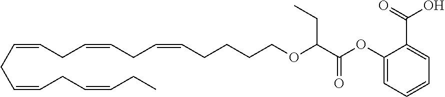 [SMILES: CC/C=C\C/C=C\C/C=C\C/C=C\C/C=C\CCCCOC(CC)C(=O)Oc1ccccc1C(=O)O]

A solution of tert-butyl 2-((2-((5Z,8Z,11Z,14Z,17Z)-icosa-5,8,11,14,17-pentaen-1-yloxy)butanoyl)oxy)benzoate (500 mg, 0.9 mmol) in FA (10 mL) was stirred for 2 days. The solvent was removed under reduced pressure and the residue was subjected to flash chromatography using a gradient of 1-5% EtOAc (containing 5% FA) in heptane (also containing 5% FA) as eluent. The appropriate fractions were pooled and concentrated and the residue was purified further by a second flash chromatography using a gradient of 1-10% EtOAc in heptane as eluent. Concentration of the appropriate fractions afforded 75 mg (17% yield) of the title compound. ¹H NMR (300 MHz, CDCl₃): δ 0.99 (t, 3H), 1.13 (t, 3H), 1.45-1.60 (m, 2H), 1.65-1.70 (m, 2H), 1.90-2.15 (m, 6H), 2.80-2.90 (m, 8H), 3.45-3.55 (m, 1H), 3.80-3.90 (m, 1H), 4.05-4.15 (m, 1H), 5.30-5.45 (m, 10H), 7.14 (d, 1H), 7.35-7.45 (m, 1H), 7.60-7.70 (m, 1H); 8.10-8.15 (m, 1H). MS (ESI): 517 [M+Na]⁺.

### Example 5

### Preparation of (2S)-ethyl 2-(2-((5Z,8Z,11Z,14Z,17Z)-icosa-5,8,11,14,17-pentaen-1-yloxy)butanamido)-4-methylpentanoate

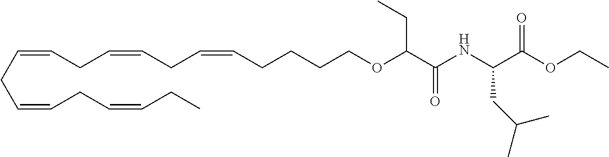 [SMILES: CC/C=C\C/C=C\C/C=C\C/C=C\C/C=C\CCCCOC(CC)C(=O)N[C@@H](CC(C)C)C(=O)OCC]

N,N′-Dicyclohexylcarbodiimide (DCC) (1.13 g, 5.5 mmol), hydroxybenzotriazole (HOBt) (0.74 g, 5.5 mmol) and triethylamine (TEA) (1.58 mL, 11.4 mmol) were added to a solution of 2-((5Z,8Z,11Z,14Z,17Z)-icosa-5,8,11,14,17-pentaen-1-yloxy)butanoic acid (1.87 g, 5.0 mmol) in tetrahydrofuran (THF) (20 mL) and the mixture was stirred for 10 minutes. L-Leucine ethyl ester hydrochloride (0.89 g, 4.6 mmol) was added and the resulting mixture was stirred for 2 hours. The mixture was concentrated in vacuo, dissolved in Et₂O (100 mL) and washed with 1M HCl (aq), saturated NaHCO₃ (aq) and brine. The organic phase was dried (Na₂SO₄), filtered and concentrated in vacuo. The residue was purified by flash chromatography using a gradient of 10-15% EtOAc in heptane as eluent. Concentration of the appropriate fractions afforded 1.88 g (80% yield) of the title compound. ¹H NMR (300 MHz, CDCl₃): δ 0.90-1.05 (m, 12H), 1.25-1.35 (m, 3H), 1.45-1.85 (m, 9H), 2.05-2.20 (m, 4H), 2.80-2.90 (m, 8H), 3.40-3.70 (m, 3H), 4.18 (q, 2H), 4.55-4.70 (m, 1H), 5.30-5.45 (m, 10H), 6.80-6.95 (m, 1H). MS (ESI): 538 [M+Na]⁺.

### Example 6

### Preparation of (2S)-2-(2-((5Z,8Z,11Z,14Z,17Z)-icosa-5,8,11,14,17-pentaen-1-yloxy)butanamido)-4-methylpentanoic acid

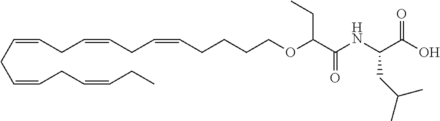 [SMILES: CC/C=C\C/C=C\C/C=C\C/C=C\C/C=C\CCCCOC(CC)C(=O)N[C@@H](CC(C)C)C(=O)O]

A solution of LiOH (700 mg, 29 mmol) in water (10 mL) was added to a solution of (2S)-ethyl 2-(2-((5Z,8Z,11Z,14Z,17Z)-icosa-5,8,11,14,17-pentaen-1-yloxy)butanamido)-4-methylpentanoate (1.88 g, 3.65 mmol) in EtOH (20 mL) and the reaction mixture was stirred at 40° C. for 1 hour. The mixture was cooled to ambient temperature and added 3M HCl (aq) until pH-2. The resulting mixture was extracted twice with Et₂O and the combined organic phases were dried (NaSO₄), filtered and concentrated under reduced pressure to afford 1.55 g (87% yield) of the title compound. ¹H NMR (300 MHz, CDCl₃): δ 0.90-1.05 (m, 12H), 1.45-1.55 (m, 2H), 1.65-1.85 (m, 7H), 2.05-2.20 (m, 4H), 2.80-2.90 (m, 8H), 3.45-3.65 (m, 2H), 3.70-3.80 (m, 1H), 4.55-4.70 (m, 1H), 5.30-5.45 (m, 10H), 6.85-7.00 (m, 1H), 10.30 (br s, 1H). MS (ESI); 510 [M+Na]⁺.

### Example 7

### Preparation of tert-butyl 2-(((2S)-2-(2-((5Z,8Z,11Z,14Z,17Z)-icosa-5,8,11,14,17-pentaen-1-yloxy)butanamido)-4-methylpentanoyl)oxy)benzoate

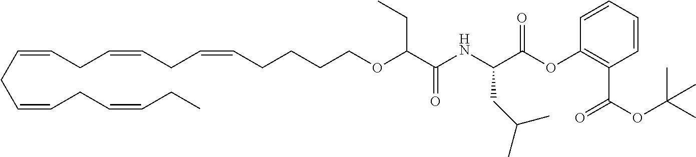 [SMILES: CC/C=C\C/C=C\C/C=C\C/C=C\C/C=C\CCCCOC(CC)C(=O)N[C@@H](CC(C)C)C(=O)Oc1ccccc1C(=O)OC(C)(C)C]

2-Hydroxy-benzoic acid tert-butyl ester (291 mg, 1.5 mmol), TEA (0.46 mL, 3.3 mmol) and O-(benzotriazol-1-yl)-N,N,N′,N′-tetramethyluronium hexafluorophosphate (TBTU) (500 mg, 1.65 mmol) were added to a solution of (2S)-2-(2-((5Z,8Z,11Z,14Z,17Z)-icosa-5,8,11,14,17-pentaen-1-yloxy)butanamido)-4-methylpentanoic acid (730 mg, 1.5 mmol) in dimethylformamide (DMF) (10 mL). The reaction mixture was heated for 2 hours at 65° C., cooled to RT and added Et₂O (100 mL). The resulting two phases were separated and the organic phase was washed with 10% NH₄Cl (aq) and brine, dried (Na₂SO₄), filtered and concentrated in vacuo. The residue was purified by flash chromatography using 10% EtOAc in heptane as eluent. Concentration of the appropriate fractions afforded 404 mg (41% yield) of the title compound. ¹H NMR (300 MHz, CDCl₃): δ 0.90-1.05 (m, 12H), 1.40-1.55 (m, 2H), 1.57 (s, 9H), 1.60-1.85 (m, 6H), 2.00-2.20 (m, 5H), 2.80-2.90 (m, 8H), 3.40-3.60 (m, 2H), 3.70-3.80 (m, 1H), 4.90-5.05 (m, 1H), 5.30-5.45 (m, 10H), 7.10 (d, 2H), 7.25-7.35 (m, 1H), 7.50-7.55 (m, 1H), 7.85-7.95 (m, 1H). MS (ESI): 686 [M+Na]⁺.

### Example 8

### Preparation of 2-(((2S)-2-(2-((5Z,8Z,11Z,14Z,17Z)-icosa-5,8,11,14,17-pentaen-1-yloxy)butanamido)-4-methylpentanoyl)oxy)benzoic acid

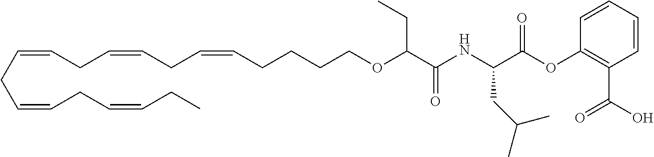 [SMILES: CC/C=C\C/C=C\C/C=C\C/C=C\C/C=C\CCCCOC(CC)C(=O)N[C@@H](CC(C)C)C(=O)Oc1ccccc1C(=O)O]

TFA (1 mL) was added to a solution of tert-butyl 2-(((2S)-2-(2-((5Z,8Z,11Z,14Z,17Z)-icosa-5,8,11,14,17-pentaen-1-yloxy)butanamido)-4-methylpentanoyl)oxy)benzoate (150 mg, 0.23 mmol) in DCM (4 mL) at 0° C. and the reaction mixture was stirred for 40 minutes. Toluene (10 mL) was added and the mixture was concentrated in vacuo. The residue was purified by flash chromatography using 13% EtOAc (containing 0.1% FA) in heptane (also containing 0.1% FA) as eluent. The appropriate fractions were concentrated and the residue (100 mg) was dissolved in Et₂O (50 mL). The solution was washed with saturated NaHCO₃ (aq), dried (Na₂SO₄), filtered and concentrated in vacuo to afford 60 mg of the desired product. ¹H NMR (300 MHz, CDCl₃): δ 0.85-1.05 (m, 12H), 1.35-2.15 (m, 13H), 2.75-2.90 (m, 8H), 3.40-3.60 (m, 2H), 3.70-3.80 (m, 1H), 4.85-5.05 (m, 1H), 5.30-5.45 (m, 10H), 7.0-7.20 (m, 2H), 7.25-7.40 (m, 1H), 7.50-7.65 (m, 1H), 8.00-8.15 (m, 1H). MS (ESI); 630 [M+Na]⁺. HR-MS (ESI): calc for C₃₇H₅₃NO₆+Na: 630.3770. found: 630.3786.

### Example 9

### Preparation of tert-butyl (2-(2-((5Z,8Z,11Z,14Z,17Z)-icosa-5,8,11,14,17-pentaen-1-yloxy)butanamido)ethyl)carbamate

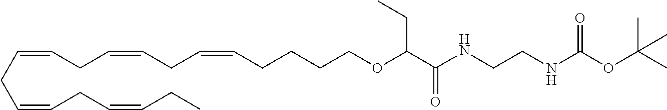 [SMILES: CC/C=C\C/C=C\C/C=C\C/C=C\C/C=C\CCCCOC(CC)C(=O)NCCNC(=O)OC(C)(C)C]

DCC (1.73 g, 8.4 mmol) and HOBt (1.14 g, 8.4 mmol) were added to a solution of 2-((5Z,8Z,11Z,14Z,17Z)-icosa-5,8,11,14,17-pentaen-1-yloxy)butanoic acid (3.14 g, 8.4 mmol) in THF (25 mL) at 0° C. and the mixture was stirred for 20 minutes. A solution of N-Boc-ethylenediamine in THF (1 mL) was added dropwise and the resulting mixture was stirred at room temperature for 1.5 hours. Et₂O (200 mL) was added and the mixture was washed with water, 1M HCl (aq), saturated NaHCO₃ (aq) and brine. The organic phase was dried (Na₂SO₄), filtered and concentrated in vacuo. The residue was purified by flash chromatography using 25% EtOAc (containing 0.5% FA) in heptane (also containing 0.5% FA) as eluent. Concentration of the appropriate fractions afforded 3.0 g (83% yield) of the title compound. ¹H NMR (300 MHz, CDCl₃): δ 0.90 (t, 3H), 0.96 (t, 3H), 1.43 (s, 9H), 1.40-1.80 (m, 6H), 2.05-2.20 (m, 4H), 2.75-2.90 (m, 8H), 3.20-3.60 (m, 6H), 3.65-3.75 (m, 1H), 5.02 (br s, 1H), 5.30-5.45 (m, 10H), 7.01 (br s, 1H). MS (ESI): 539 [M+Na]⁺.

### Example 10

### Preparation of 2-hydroxy-N-(2-(2-((5Z,8Z,11Z,14Z,17Z)-icosa-5,8,11,14,17-pentaen-1-yloxy)butanamido)ethyl)benzamide

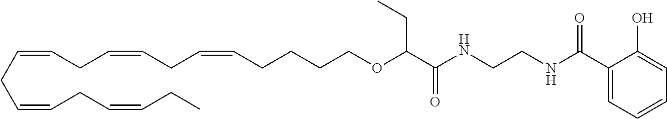 [SMILES: CC/C=C\C/C=C\C/C=C\C/C=C\C/C=C\CCCCOC(CC)C(=O)NCCNC(=O)c1ccccc1O]

TFA (2 mL) was added to a solution of tert-butyl (2-(2-((5Z,8Z,11Z,14Z,17Z)-icosa-5,8,11,14,17-pentaen-1-yloxy)butanamido)ethyl)carbamate (668 mg, 1.3 mmol) in DCM (8 mL) and the resulting mixture was stirred for 1.5 hours. The mixture was concentrated in vacuo to give the TFA salt of N-(2-aminoethyl)-2-((5Z,8Z,11Z,14Z,17Z)-icosa-5,8,11,14,17-pentaen-1-yloxy)butanamide as a crude product (800 mg, MS (ESI): 417 [M+H]⁺, 439 [M+Na]⁺). The residue was dissolved in DCM (10 mL) and added DCC (320 mg, 1.55 mmol), TEA (0.36 mL, 2.6 mmol) and HOBt (210 mg, 1.55 mmol). After 15 minutes, a solution of salicylic acid (214 mg, 1.55 mmol) in DCM (1 mL) was added dropwise and the resulting mixture was stirred over night. The mixture was concentrated and the residue was dissolved in Et₂O (100 mL), washed with water, 1M HCl (aq), saturated NaHCO₃ (aq) and brine. The organic phase was dried (Na₂SO₄), filtered and concentrated in vacuo. The crude oil was purified by flash chromatography using a gradient of 4-15% EtOAc (containing 5% FA) in heptane (also containing 5% FA) as eluent. Concentration of the appropriate fractions afforded 110 mg (16% yield over two steps) of the title compound. ¹H NMR (300 MHz, CDCl₃): δ 0.89 (t, 3H), 0.99 (t, 3H), 1.35-1.50 (m, 2H), 1.60-1.90 (m, 4H), 2.05-2.20 (m, 4H), 2.8-2.9 (m, 8H), 3.47 (t, 2H), 3.50-3.70 (m, 4H), 3.70-3.80 (m, 1H), 5.30-5.45 (m, 10H), 6.85-6.95 (m, 1H), 6.97 (dd, 1H), 7.14 (br s, 1H), 7.35-7.45 (m, 1H), 7.48 (dd, 1H), 7.95 (br s, 1H), 12.51 (br s, 1H). MS (ESI): 537 [M+H]⁺, 559 [M+Na]⁺.

### Example 11

### Preparation of 2-((tert-butoxycarbonyl)amino)ethyl 2-((5Z,8Z,11Z,14Z,17Z)-icosa-5,8,11,14,17-pentaen-1-yloxy)butanoate

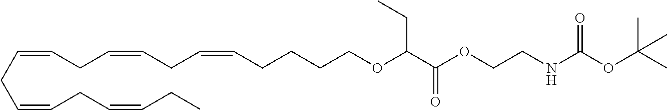 [SMILES: CC/C=C\C/C=C\C/C=C\C/C=C\C/C=C\CCCCOC(CC)C(=O)OCCNC(=O)OC(C)(C)C]

O-(7-Azabenzotriazol-1-yl)-N,N,N′,N′-tetramethyluronium hexafluorophosphate (HATU) (800 mg, 2.1 mmol) and TEA (0.56 mL, 4 mmol) were added to a solution of 2-((5Z,8Z,11Z,14Z,17Z)-icosa-5,8,11,14,17-pentaen-1-yloxy)butanoic acid (748 mg, 2 mmol) in DCM (10 mL) and the reaction mixture was stirred for 20 minutes. A solution of tert-butyl N-(2-hydroxyethyl)carbamate (340 mg, 2.1 mmol) in DCM (1 mL) was added and the resulting mixture was stirred at room temperature over night. Et₂O (100 mL) was added and the mixture was washed with water, 1M HCl (aq), saturated NaHCO₃ (aq) and brine, dried (Na₂SO₄), filtered and concentrated in vacuo. The residue was purified by flash chromatography using a gradient of 10-20% EtOAc in heptane as eluent. Concentration of the appropriate fractions afforded 780 mg (76% yield) of the title compound. ¹H NMR (300 MHz, CDCl₃): δ 0.98 (t, 6H), 1.40-1.55 (m, 2H), 1.45 (s, 9H), 1.60-1.85 (m, 4H), 2.05-2.20 (m, 4H), 2.75-2.90 (m, 8H), 3.30-3.45 (m, 3H), 3.55-3.65 (m, 1H), 3.75-3.80 (m, 1H), 4.20-4.25 (m, 2H), 4.76 (br s, 1H), 5.30-5.45 (m, 10H). MS (ESI): 540 [M+Na]⁺.

### Example 12

### Preparation of 2-(2-acetoxybenzamido)ethyl 2-((5Z,8Z,11Z,14Z,17Z)-icosa-5,8,11,14,17-pentaen-1-yloxy)butanoate

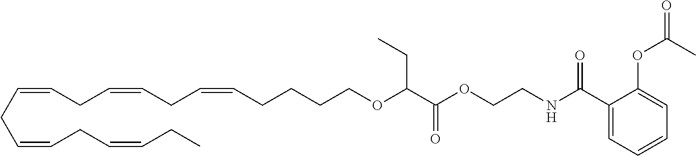 [SMILES: CC/C=C\C/C=C\C/C=C\C/C=C\C/C=C\CCCCOC(CC)C(=O)OCCNC(=O)c1ccccc1OC(C)=O]

Acetyl chloride (1 mL) was added to a solution of 2-((tert-butoxycarbonyl)amino)ethyl 2-((5Z,8Z,11Z,14Z,17Z)-icosa-5,8,11,14,17-pentaen-1-yloxy)butanoate (300 mg, 0.58 mmol) in MeOH (5 mL) at 0° C. and the reaction mixture was stirred at room temperature for 1 hour. The mixture was concentrated in vacuo to afford the HCl salt of 2-aminoethyl 2-((5Z,8Z,11Z,14Z,17Z)-icosa-5,8,11,14,17-pentaen-1-yloxy)butanoate (286 mg) as a crude product. TEA (109 μL, 0.78 mmol) was added to a solution of acetyl salicylic acid (137 mg, 0.76 mmol) in DCM (10 mL) and the mixture was cooled to 0° C. Ethyl chloroformate (75 μL, 0.78 mmol) was added drop wise and the reaction mixture was stirred for 2 hours. This solution was then added to a solution of the crude product of the HCl salt of 2-aminoethyl 2-((5Z,8Z,11Z,14Z,17Z)-icosa-5,8,11,14,17-pentaen-1-yloxy)butanoate in a mixture of DCM (10 mL) and TEA (2 mL). The resulting mixture was stirred at room temperature for 3.5 hours and then added water. The resulting two phases were separated and the aqueous phase was extracted twice with DCM. The combined organic phases were washed with 1M HCl (aq), saturated NaHCO₃ (aq) and water, dried (Na₂SO₄), filtered and concentrated in vacuo. The residue was purified by flash chromatography using 20% EtOAc in heptane as eluent. Concentration of the appropriate fractions afforded 90 mg (23% yield) of the title compound. ¹H NMR (300 MHz, CDCl₃): δ 0.95-1.05 (m, 6H), 1.35-1.45 (m, 2H), 1.55-1.70 (m, 2H), 1.70-1.85 (m, 2H), 2.05-2.15 (m, 4H), 2.35 (s, 3H), 2.80-2.90 (m, 8H), 3.30-3.40 (m, 1H), 3.55-3.65 (m, 1H), 3.70-3.85 (m, 3H), 4.33 (t, 2H), 5.30-5.45 (m, 10H), 6.61 (br s, 1H), 7.10-7.15 (m, 1H), 7.25-7.35 (m, 1H), 7.45-7.50 (m, 1H), 7.70-7.75 (m, 1H). MS (ESI); 602 [M+Na]⁺.

### Example 13

### Preparation of 2-(2-hydroxybenzamido)ethyl 2-((5Z,8Z,11Z,14Z,17Z)-icosa-5,8,11,14,17-pentaen-1-yloxy)butanoate

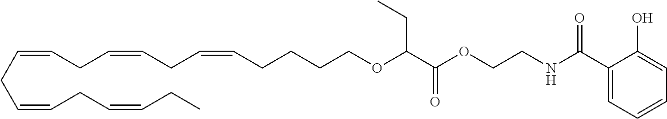 [SMILES: CC/C=C\C/C=C\C/C=C\C/C=C\C/C=C\CCCCOC(CC)C(=O)OCCNC(=O)c1ccccc1O]

Ammonia (aq, 28%, 20 drops) was added dropwise to a solution of 2-(2-acetoxybenzamido)ethyl 2-((5Z,8Z,11Z,14Z,17Z)-icosa-5,8,11,14,17-pentaen-1-yloxy)butanoate (99 mg, 0.17 mmol) in 2-propanol (9 mL) and water (3 mL) and the reaction mixture was stirred for 10 minutes. Water was added and the resulting mixture was extracted twice with Et₂O. The combined organic phases were washed with brine, dried (Na₂SO₄), filtered and concentrated in vacuo. The residue was purified by flash chromatography using a gradient of 0-40% EtOAc in heptane as eluent. Concentration of the appropriate fractions afforded 33 mg (36% yield) of the title compound. ¹H NMR (300 MHz, CDCl₃): δ 0.95-1.05 (m, 6H), 1.35-1.45 (m, 2H), 1.60-1.70 (m, 2H), 1.70-1.85 (m, 2H), 2.05-2.20 (m, 4H), 2.80-2.90 (m, 8H), 3.35-3.45 (m, 1H), 3.50-3.60 (m, 1H), 3.75-3.85 (m, 3H), 4.40-4.50 (m, 2H), 5.30-5.45 (m, 10H), 6.80-6.90 (m, 2H), 6.95-7.05 (m, 1H), 7.35-7.45 (m, 1H), 12.22 (s, 1H). MS (ESI): 560 [M+Na]⁺.

### Example 14

### Preparation of N-(2-hydroxyethyl)-2-((5Z,8Z,11Z,14Z,17Z)-icosa-5,8,11,14,17-pentaen-1-yloxy)butanamide

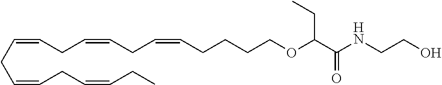 [SMILES: CC/C=C\C/C=C\C/C=C\C/C=C\C/C=C\CCCCOC(CC)C(=O)NCCO]

Oxalyl chloride (8.4 mL, 100 mmol) followed by 2 drops of DMF were added to a solution of 2-((5Z,8Z,11Z,14Z,17Z)-icosa-5,8,11,14,17-pentaen-1-yloxy)butanoic acid (4.5 g, 12 mmol) in DCM (100 mL). The mixture was stirred for 30 minutes and then concentrated under reduced pressure. The residue was dissolved in DCM (100 mL) and added TEA (3.34 mL, 24 mmol) and ethanolamine (1.08 mL, 18 mmol). After 2 hours, water (300 mL) was added and the resulting mixture was extracted twice with DCM. The combined organic phases were dried (Na₂SO₄), filtered and concentrated in vacuo. The residue was purified by flash chromatography using a gradient of 20-50% EtOAc in heptane as eluent. Concentration of the appropriate fractions afforded 4.6 g (92% yield) of the title compound. ¹H NMR (300 MHz, CDCl₃): δ 0.90-1.05 (m, 6H), 1.40-1.55 (m, 2H), 1.60-1.90 (m, 4H), 2.05-2.20 (m, 4H), 2.75-2.90 (m, 8H), 3.05 (br s, 1H), 3.40-3.55 (m, 4H), 3.70-3.80 (m, 3H), 5.30-5.45 (m, 10H), 7.04 (br s, 1H). MS (ESI); 440 [M+Na]⁺.

### Example 15

### Preparation of 2-(2-((5Z,8Z,11Z,14Z,17Z)-icosa-5,8,11,14,17-pentaen-1-yloxy)butanamido)ethyl 2-acetoxybenzoate

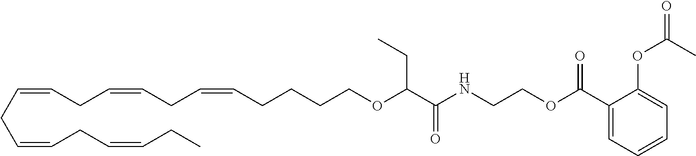 [SMILES: CC/C=C\C/C=C\C/C=C\C/C=C\C/C=C\CCCCOC(CC)C(=O)NCCOC(=O)c1ccccc1OC(C)=O]

TBTU (0.85 g, 2.64 mmol) and TEA (0.8 mL, 5.3 mmol) were added to a solution of acetylsalicylic acid (0.4 g, 2.4 mmol) in DCM (20 mL) and the mixture was stirred for 10 minutes. A solution of N-(2-hydroxyethyl)-2-((5Z,8Z,11Z,14Z,17Z)-icosa-5,8,11,14,17-pentaen-1-yloxy)butanamide (1.0 g, 1.4 mmol) in DCM (10 mL) was added and the reaction mixture was stirred at room temperature for 1 hour and then refluxed overnight. Water was added and the resulting mixture was extracted twice with DCM. The combined organic phases were dried (Na₂SO₄), filtered and concentrated in vacuo. The residue was purified by flash chromatography using a gradient of 0-20% EtOAc in heptane as eluent. Concentration of the appropriate fractions afforded 900 mg (65% yield) of the title compound. ¹H NMR (300 MHz, CDCl₃): δ 0.85-1.00 (m, 6H), 1.35-1.50 (m, 2H), 1.50-1.85 (m, 4H), 2.00-2.20 (m, 4H), 2.37 (s, 3H), 2.75-2.90 (m, 8H), 3.40-3.55 (m, 2H), 3.55-3.75 (m, 3H), 4.15-4.45 (m, 2H), 5.30-5.50 (m, 10H), 6.80-95 (m, 1H), 7.10-7.15 (m, 1H), 7.30-7.40 (m, 1H), 7.55-7.65 (m, 1H), 8.00-8.05 (m, 1H). MS (ESI): 602 [M+Na]⁺.

### Example 16

### Preparation of 2-(2-((5Z,8Z,11Z,14Z,17Z)-icosa-5,8,11,14,17-pentaen-1-yloxy)butanamido)ethyl 2-hydroxybenzoate

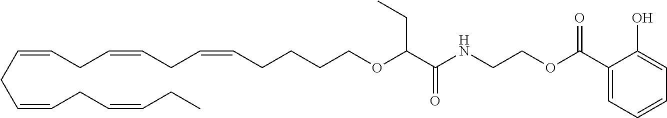 [SMILES: CC/C=C\C/C=C\C/C=C\C/C=C\C/C=C\CCCCOC(CC)C(=O)NCCOC(=O)c1ccccc1O]

Ammonia (aq, 28%, 3 mL) was added dropwise to a solution of 2-(2-((5Z,8Z,11Z,14Z,17Z)-icosa-5,8,11,14,17-pentaen-1-yloxy)butanamido)ethyl 2-acetoxybenzoate (390 mg, 0.67 mmol) in 2-propanol (27 mL) and water (9 mL) and the mixture was stirred for 10 minutes. Water was added and the resulting mixture was extracted twice with Et₂O. The combined organic phases were washed with brine, dried (Na₂SO₄), filtered and concentrated in vacuo. The residue was purified by flash chromatography using a gradient of 0-20% EtOAc in heptane as eluent. Concentration of the appropriate fractions afforded 63 mg (18% yield) of the title compound. ¹H NMR (300 MHz, CDCl₃): δ 0.91 (t, 3H), 0.97 (t, 3H), 1.35-1.45 (m, 2H), 1.55-1.90 (m, 4H), 2.00-2.15 (m, 4H), 2.80-2.90 (m, 8H), 3.44 (t, 2H), 3.65-3.80 (m, 3H), 4.40-4.50 (m, 2H), 5.30-5.45 (m, 10H), 6.85-6.95 (m, 2H), 6.95-7.05 (m, 1H), 7.40-7.50 (m, 1H), 7.80-7.85 (m, 1H), 10.63 (s, 1H). MS (ESI): 560 [M+Na]⁺.

### Example 17

### Preparation of methyl 2-hydroxy-5-(2-((5Z,8Z,11Z,14Z,17Z)-icosa-5,8,11,14,17-pentaen-1-yloxy)butanamido)benzoate

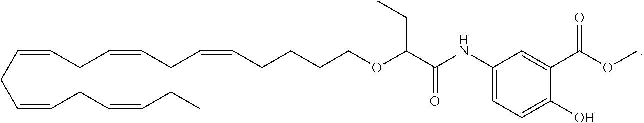 [SMILES: CC/C=C\C/C=C\C/C=C\C/C=C\C/C=C\CCCCOC(CC)C(=O)Nc1ccc(O)c(C(=O)OC)c1]

2-((5Z,8Z,11Z,14Z,17Z)-Icosa-5,8,11,14,17-pentaen-1-yloxy)butanoic acid (300 mg, 0.80 mmol), methyl 5-aminosalicylate (140 mg, 0.83 mmol) and TEA (0.22 mL, 1.60 mmol) was dissolved in MeCN (3 mL). HATU (320 mg, 0.83 mmol) was added and the reaction mixture was stirred over night. The mixture was then concentrated under reduced pressure and the residue was partitioned between Et₂O (30 mL) and brine (20 mL). The aqueous phase was extracted with Et₂O (20 mL) and the combined organic phases were washed with 2M HCl (aq, 15 mL), saturated NaHCO₃ (aq, 15 mL) and brine (15 mL), dried (Na₂SO₄), filtered and concentrated in vacuo. The residue was purified by flash chromatography using 5% EtOAc in heptane as eluent. Concentration of the appropriate fractions afforded 320 mg (77% yield) of the title compound. MS (ESI): 546 [M+Na]⁺.

### Example 18

### Preparation of 2-hydroxy-5-(2-((5Z,8Z,11Z,14Z,17Z)-icosa-5,8,11,14,17-pentaen-1-yloxy)butanamido)benzoic acid

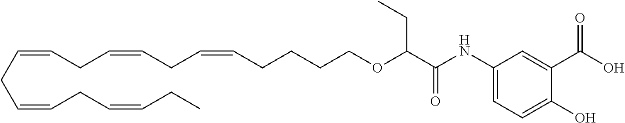 [SMILES: CC/C=C\C/C=C\C/C=C\C/C=C\C/C=C\CCCCOC(CC)C(=O)Nc1ccc(O)c(C(=O)O)c1]

2M NaOH (aq, 6 mL) was added to a solution of methyl 2-hydroxy-5-(2-((5Z,8Z,11Z,14Z,17Z)-icosa-5,8,11,14,17-pentaen-1-yloxy)butanamido)benzoate (320 mg, 0.61 mmol) in MeOH (3 mL) and the reaction mixture was heated at 50° C. over night. The mixture was cooled to ambient temperature and acidified to pH-2 with 5M HCl (aq). The resulting mixture was extracted with EtOAc and the organic phase was dried (Na₂SO₄), filtered and concentrated in vacuo. The residue was purified by flash chromatography using a gradient of 1-2% MeOH in EtOAc as eluent. Concentration of the appropriate fractions afforded 85 mg (27% yield) of the title compound. ¹H NMR (300 MHz, CDCl₃): δ 0.90-1.05 (t, 3H), 1.20-1.30 (m, 1H), 1.45-1.60 (m, 2H), 1.65-1.75 (m, 2H), 1.80-1.95 (m, 2H), 2.05-2.20 (m, 4H), 2.80-2.90 (m, 8H), 3.50-3.65 (m, 2H), 3.85-3.95 (m, 1H), 5.30-5.45 (m, 10H), 6.90-7.00 (m, 1H), 7.58 (br s, 1H), 8.11 (br s, 1H), 8.40 (s, 1H). MS (ESI): 508 [M−H]⁻.

### Example 19

### Preparation of (2S)-ethyl 2-(2-((5Z,8Z,11Z,14Z,17Z)-icosa-5,8,11,14,17-pentaen-1-yloxy)butanamido)-4-methylpentanoate

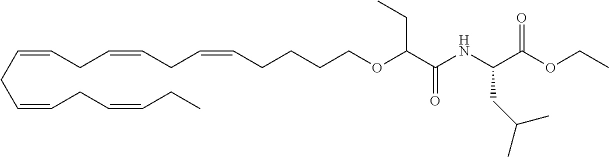 [SMILES: CC/C=C\C/C=C\C/C=C\C/C=C\C/C=C\CCCCOC(CC)C(=O)N[C@@H](CC(C)C)C(=O)OCC]

DCC (1.13 g, 5.5 mmol) and HOBt (0.74 g, 5.5 mmol) followed by TEA (1.58 mL, 11.4 mmol) were added to a solution of 2-((5Z,8Z,11Z,14Z,17Z)-icosa-5,8,11,14,17-pentaen-1-yloxy)butanoic acid (1.87 g, 5.0 mmol) in THF (20 mL) and the mixture was stirred for 10 minutes. L-Leucine ethyl ester hydrochloride (0.89 g, 4.6 mmol) was added and the resulting mixture was stirred for 2 hours. EtOAc (100 mL) was added and the mixture was washed with water, 1M HCl (aq), saturated NaHCO₃ (aq) and brine. The organic phase was dried (Na₂SO₄), filtered and concentrated in vacuo. The residue was purified by flash chromatography using a gradient of 10-15% EtOAc in heptane as eluent. Concentration of the appropriate fractions afforded 1.84 g (79% yield) of the title compound. ¹H NMR (300 MHz, CDCl₃): δ 0.85-1.05 (m, 12H), 1.25-1.35 (m, 3H), 1.40-1.85 (m, 9H), 2.05-2.20 (m, 4H), 2.80-2.90 (m, 8H), 3.45-3.75 (m, 3H), 4.15-4.25 (m, 2H), 4.55-4.75 (m, 1H), 5.30-5.45 (m, 10H), 6.80-6.95 (m, 1H). MS (ESI): 538 [M+Na]⁺.

### Example 20

### Preparation of (2S)-2-(2-((5Z,8Z,11Z,14Z,17Z)-icosa-5,8,11,14,17-pentaen-1-yloxy)butanamido)-4-methylpentanoic acid

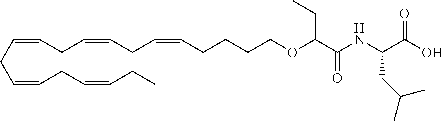 [SMILES: CC/C=C\C/C=C\C/C=C\C/C=C\C/C=C\CCCCOC(CC)C(=O)N[C@@H](CC(C)C)C(=O)O]

1M LiOH (aq, 28 mL) was added to a solution of (2S)-ethyl 2-(2-((5Z,8Z,11Z,14Z,17Z)-icosa-5,8,11,14,17-pentaen-1-yloxy)butanamido)-4-methylpentanoate (1.79 g, 3.5 mmol) in EtOH (50 mL) and the reaction mixture was heated to 50° C. for 2 hours. The mixture was cooled to ambient temperature and added 6M HCl (aq) to pH-2. The resulting mixture was extracted with twice with Et₂O and the combined organic phases were dried (NaSO₄), filtered and concentrated under reduced pressure to afford 1.61 g (95% yield) of the title compound. ¹H NMR (300 MHz, CDCl₃): δ 0.85-1.05 (m, 12H), 1.45-1.55 (m, 2H), 1.60-1.85 (m, 7H), 2.05-2.20 (m, 4H), 2.80-2.90 (m, 8H), 3.45-3.65 (m, 2H), 3.70-3.80 (m, 1H), 4.60-4.75 (m, 1H), 5.30-5.45 (m, 10H), 6.90-7.05 (m, 1H), 10.15 (br s, 1H). MS (ESI): 486 [M−H]⁻.

### Example 21

### Preparation of methyl 2-hydroxy-5-((2S)-2-(2-((5Z,8Z,11Z,14Z,17Z)-icosa-5,8,11,14,17-pentaen-1-yloxy)butanamido)-4-methylpentanamido)benzoate

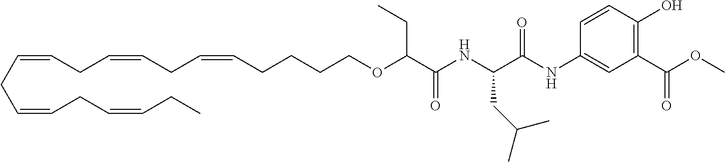 [SMILES: CC/C=C\C/C=C\C/C=C\C/C=C\C/C=C\CCCCOC(CC)C(=O)N[C@@H](CC(C)C)C(=O)Nc1ccc(O)c(C(=O)OC)c1]

DCC (248 mg, 1.2 mmol) and HOBt (163 mg, 1.2 mmol) were added to a solution of (2S)-2-(2-((5Z,8Z,11Z,14Z,17Z)-icosa-5,8,11,14,17-pentaen-1-yloxy)butanamido)-4-methylpentanoic acid (487 mg, 1.0 mmol) in THF (8 mL) at 0° C. A solution of methyl 5-amino salicylate (201 mg, 1.2 mmol) in THF (1 mL) was added dropwise and the reaction mixture was stirred at room temperature for 3 hours. Et₂O (100 mL) was added and the mixture was washed with water, 1M HCl (aq), saturated NaHCO₃ (aq) and brine. The organic phase was dried (Na₂SO₄), filtered and concentrated in vacuo. The residue was purified by flash chromatography using a gradient of 3-15% EtOAc (containing 5% FA) in heptane (also containing 5% FA) as eluent. Concentration of the appropriate fractions afforded 197 mg (31% yield) of the title compound. ¹H NMR (300 MHz, CDCl₃): δ 0.85-1.05 (m, 12H), 1.40-1.50 (m, 2H), 1.60-1.90 (m, 7H), 2.05-2.20 (m, 4H), 2.80-2.90 (m, 8H), 3.45-3.55 (m, 2H), 3.75-3.80 (m, 1H), 3.94 (s, 1H), 4.55-4.65 (m, 1H), 5.30-5.45 (m, 10H), 6.90-7.00 (m, 2H), 7.45-7.50 (m, 1H), 8.00-8.10 (m, 1H), 8.66 (s, 1H), 10.59 (s, 1H). MS (ESI): 659 [M+Na]⁺.

### Example 22

### Preparation of 2-hydroxy-5-((2S)-2-(2-((5Z,8Z,11Z,14Z,17Z)-icosa-5,8,11,14,17-pentaen-1-yloxy)butanamido)-4-methylpentanamido)benzoic acid

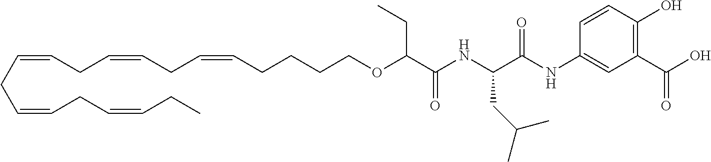 [SMILES: CC/C=C\C/C=C\C/C=C\C/C=C\C/C=C\CCCCOC(CC)C(=O)N[C@@H](CC(C)C)C(=O)Nc1ccc(O)c(C(=O)O)c1]

1M LiOH (aq, 2.5 mL) was added to a solution of methyl 2-hydroxy-5-((2S)-2-(2-((5Z,8Z,11Z,14Z,17Z)-icosa-5,8,11,14,17-pentaen-1-yloxy)butanamido)-4-methylpentanamido)benzoate (190 mg, 0.3 mmol) in MeOH (5 mL). The reaction mixture was heated at 50° C. for 5 hours and then stirred at room temperature for 3 days. The mixture was acidified with 6M HCl (aq) to pH-2 and most of the solvent was removed in vacuo. The residue was extracted twice with Et₂O and the combined organic phases were dried (Na₂SO₄), filtered and concentrated in vacuo. The residue was purified by flash chromatography using a gradient of 5-25% EtOAc (containing 5% FA) in heptane (also containing 5% FA) as eluent. Concentration of the appropriate fractions afforded 100 mg (54% yield) of the title compound. ¹H NMR (300 MHz, CDCl₃): δ 0.90-1.05 (m, 12H), 1.40-1.50 (m, 2H), 1.60-1.90 (m, 7H), 2.05-2.20 (m, 4H), 2.80-2.90 (m, 8H), 3.45-3.60 (m, 2H), 3.80-3.90 (m, 1H), 4.60-4.75 (m, 1H), 5.30-5.45 (m, 10H), 6.89 (d, 1H), 7.24 (d, 1H), 7.70-7.90 (m, 2H), 9.02 (d, 1H), 10.37 (d, 1H). MS (ESI): 623 [M+H]⁺.

### Example 23

### Preparation of methyl 2-hydroxy-5-(((2-(2-((5Z,8Z,11Z,14Z,17Z)-icosa-5,8,11,14,17-pentaen-1-yloxy)butanamido)ethoxy)carbonyl)amino)benzoate

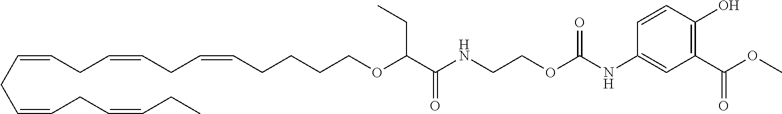 [SMILES: CC/C=C\C/C=C\C/C=C\C/C=C\C/C=C\CCCCOC(CC)C(=O)NCCOC(=O)Nc1ccc(O)c(C(=O)OC)c1]

Diphosgene (0.16 mL, 1.3 mmol) and diisopropylamine (0.16 mL, 0.96 mmol) were added to a solution of N-(2-hydroxyethyl)-2-((5Z,8Z,11Z,14Z,17Z)-icosa-5,8,11,14,17-pentaen-1-yloxy)butanamide (0.4 g, 0.96 mmol) in DCM (15 mL) at 0° C. The reaction mixture was stirred at 0° C. for 1 hour and then at room temperature for 2 hours. The mixture was concentrated under reduced pressure and the residue was suspended in THF (20 mL). The suspension was filtered and the filtrate was concentrated under reduced pressure. The residue was then suspended in Et₂O, filtered through a pad of silica and concentrated in vacuo to afford 0.66 g of 2-(2-((5Z,8Z,11Z,14Z,17Z)-icosa-5,8,11,14,17-pentaen-1-yloxy)butanamido)ethyl carbonochloridate as a crude product. The residue was dissolved in DCM (15 mL) and added methyl 5-aminosalicylate (0.13 g, 0.77 mmol) and TEA (0.2 mL, 1.54 mmol). The reaction mixture was stirred for 2 hours and then added water. The resulting mixture was extracted twice with DCM and the combined organic phases were dried (Na₂SO₄), filtered and concentrated in vacuo. The residue was purified by flash chromatography using a gradient of 30-50% EtOAc in heptane as eluent. Concentration of the appropriate fractions afforded 145 mg (30% yield) of the title compound. ¹H NMR (300 MHz, CDCl₃): δ 0.85-1.00 (m, 6H), 1.35-1.50 (m, 2H), 1.50-1.85 (m, 4H), 2.00-2.20 (m, 4H), 2.75-2.90 (m, 8H), 3.40-3.55 (m, 2H), 3.55-3.75 (m, 3H), 3.95 (s, 3H), 4.25-4.35 (m, 2H), 5.25-5.45 (m, 10H), 6.68 (br s, 1H), 6.85-7.00 (m, 2H), 7.35-7.45 (m, 1H), 7.91 (br s, 1H), 10.57 (s, 1H). MS (ESI): 633 [M+Na]⁺.

### Example 24

### Preparation of 2-hydroxy-5-(((2-(2-((5Z,8Z,11Z,14Z,17Z)-icosa-5,8,11,14,17-pentaen-1-yloxy)butanamido)ethoxy)carbonyl)amino)benzoic acid

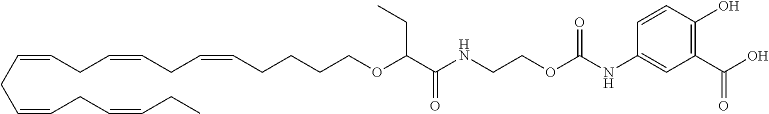 [SMILES: CC/C=C\C/C=C\C/C=C\C/C=C\C/C=C\CCCCOC(CC)C(=O)NCCOC(=O)Nc1ccc(O)c(C(=O)O)c1]

1M LiOH (aq, 1.9 mL) was added to a solution of methyl 2-hydroxy-5-(((2-(2-((5Z,8Z,11Z,14Z,17Z)-icosa-5,8,11,14,17-pentaen-1-yloxy)butanamido)ethoxy)carbonyl)amino)benzoate (145 mg, 0.24 mmol) in MeOH (20 mL) and the mixture was heated at 50° C. over night. The reaction mixture was acidified with 1M HCl (aq) to pH-2 and then extracted twice with Et₂O. The combined organic phases were dried (Na₂SO₄), filtered and concentrated in vacuo. The residue was purified by flash chromatography using 20% EtOAc (containing 5% FA) in heptane (also containing 5% FA) as eluent. The appropriate fractions were pooled and concentrated. The residue (46 mg) was purified further by preparative HPLC using a gradient of 30-95% MeCN in water (containing 5% MeCN and 0.01% TFA) as eluent. The appropriate fractions were pooled and concentrated and the residue was dissolved in toluene. Concentration of the solution afforded 24 mg (17% yield) of the title compound. ¹H NMR (300 MHz, CDCl₃): δ 0.91-1.01 (m, 6H), 1.35-1.50 (m, 2H), 1.55-1.90 (m, 4H), 2.05-2.15 (m, 4H), 2.80-2.90 (m, 8H), 3.45-3.55 (m, 2H), 3.65-3.75 (m, 2H), 3.75-3.85 (m, 1H), 4.25-4.35 (m, 2H), 5.30-5.45 (m, 10H), 6.90-7.00 (m, 2H), 7.00-7.10 (m, 1H), 7.63 (br s, 1H), 7.83 (s, 1H), 10.43 (br s, 1H). MS (ESI): 597 [M+H]⁺.

### Example 25

### Preparation of N-(2-aminoethyl)-2-((5Z,8Z,11Z,14Z,17Z)-icosa-5,8,11,14,17-pentaen-1-yloxy)butanamide

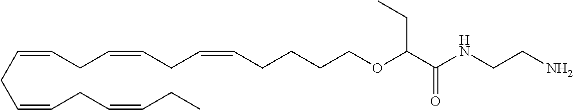 [SMILES: CC/C=C\C/C=C\C/C=C\C/C=C\C/C=C\CCCCOC(CC)C(=O)NCCN]

TFA (2 mL) was added to a solution of the tert-butyl (2-(2-((5Z,8Z,11Z,14Z,17Z)-icosa-5,8,11,14,17-pentaen-1-yloxy)butanamido)ethyl)carbamate (774 mg, 1.5 mmol) in DCM (8 mL). The reaction mixture was stirred for 30 minutes and then concentrated in vacuo. Et₂O (50 mL) and 1M NaOH (aq, 50 mL) was added to the residue and the mixture was stirred vigorously for 30 minutes. The phases were separated and the organic phase was washed with brine, dried (NaSO₄), filtered and concentrated under reduced pressure to afford 560 mg (90% yield) of the title compound. ¹H NMR (300 MHz, CDCl₃): δ 0.85-1.05 (2×t, 6H), 1.35-1.50 (m, 2H), 1.55-1.85 (m, 4H), 2.05-2.20 (m, 4H), 2.75-2.95 (m, 8H), 3.10-3.20 (m, 1H), 3.30-3.80 (m, 6H), 4.05-4.20 (br m, 1H), 4.25-4.75 (br s, 2H), 5.30-5.50 (m, 10H). MS (ESI): 417 [M+H]⁺, 439 [M+Na]⁺.

### Example 26

### Preparation of 2-((5Z,8Z,11Z,14Z,17Z)-icosa-5,8,11,14,17-pentaen-1-yloxy)-N-(2-isocyanatoethyl)butanamide

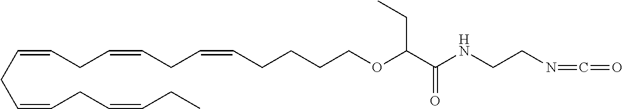 [SMILES: CC/C=C\C/C=C\C/C=C\C/C=C\C/C=C\CCCCOC(CC)C(=O)NCCN=C=O]

1,8-Bis(dimethylamino)naphthalene (577 mg, 2.7 mmol) was added to a solution of N-(2-aminoethyl)-2-(5Z,8Z,11Z,14Z,17Z)-icosa-5,8,11,14,17-pentaen-1-yloxy)butanamide (560 mg, 1.35 mmol) in DCM (10 mL) and the mixture was cooled to 0° C. Trichloromethyl chloroformate (98 μL, 0.81 mmol) was added dropwise before the cooling bath was removed and the mixture was stirred for 15 minutes. 1M HCl (aq, 30 mL) and DCM (30 mL) was added. The resulting two phases were separated and the organic phase was washed 4 times with 1M HCl (aq) and once with 1M NaOH (aq), dried (NaSO₄), filtered and concentrated under reduced pressure to afford 500 mg (84% yield) of the title compound as a crude product. MS (ESI): 465 [M+Na]⁺.

### Example 27

### Preparation of methyl 2-hydroxy-5-(3-(2-(2-((5Z,8Z,11Z,14Z,17Z)-icosa-5,8,11,14,17-pentaen-1-yloxy)butanamido)ethyl)ureido)benzoate

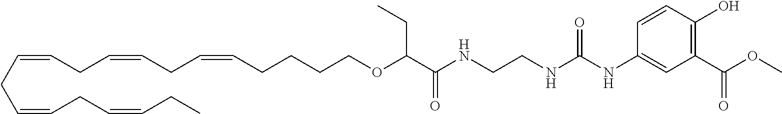 [SMILES: CC/C=C\C/C=C\C/C=C\C/C=C\C/C=C\CCCCOC(CC)C(=O)NCCNC(=O)Nc1ccc(O)c(C(=O)OC)c1]

Methyl 5-aminosalicyate (189 mg, 1.13 mmol) was added to a solution of 2-((5Z,8Z,11Z,14Z,17Z)-icosa-5,8,11,14,17-pentaen-1-yloxy)-N-(2-isocyanatoethyl)butanamide (500 mg, 1.13 mmol) in DCM (5 mL). The reaction mixture was stirred for 2 hours and then concentrated in vacuo. The residue was purified by flash chromatography using a gradient of 40-0% heptane in EtOAc as eluent. Concentration of the appropriate fractions afforded 230 mg (33% yield) of the title compound. ¹H NMR (300 MHz, CDCl₃): δ 0.85-1.05 (2×t, 6H), 1.35-1.50 (m, 2H), 1.55-1.85 (m, 4H), 2.05-2.20 (m, 4H), 2.75-2.95 (m, 8H), 3.35-3.50 (m, 6H), 3.65-3.75 (m, 1H), 3.94 (s, 3H), 5.30-5.50 (m, 10H), 6.95 (d, 1H), 7.10 (br s, 1H), 7.40 (dd, 1H), 7.88 (d, 1H), 10.62 (s, 1H). MS (ESI); 632 [M+Na]⁺.

### Example 28

### Preparation of 2-hydroxy-5-(3-(2-(2-((5Z,8Z,11Z,14Z,17Z)-icosa-5,8,11,14,17-pentaen-1-yloxy)butanamido)ethyl)ureido)benzoic acid

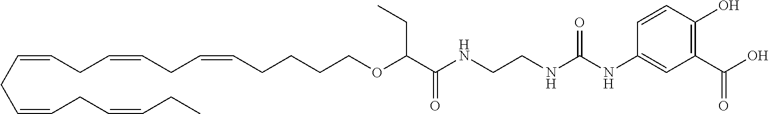 [SMILES: CC/C=C\C/C=C\C/C=C\C/C=C\C/C=C\CCCCOC(CC)C(=O)NCCNC(=O)Nc1ccc(O)c(C(=O)O)c1]

1M LiOH (aq, 2.9 mL) was added dropwise to a solution of methyl 2-hydroxy-5-(3-(2-(2-((5Z,8Z,11Z,14Z,17Z)-icosa-5,8,11,14,17-pentaen-1-yloxy)butanamido)ethyl)ureido)benzoate (220 mg, 0.36 mmol) in MeOH (10 mL) and the mixture was heated at 50° C. over night. The mixture was cooled to ambient temperature and then acidified to pH-2 with 6M HCl (aq). The resulting mixture was extracted twice with EtOAc and the combined organic phases were washed with brine, dried (NaSO₄), filtered and concentrated under reduced pressure. The residue was purified by flash chromatography using a gradient of 5-40% EtOAc (containing 5% FA) in heptane (also containing 5% FA) as eluent. Concentration of the appropriate fractions afforded 92 mg (43% yield) of the title compound. ¹H NMR (300 MHz, CDCl₃): δ 0.91 (t, 3H), 0.98 (t, 3H), 1.40-1.50 (m, 2H), 1.60-1.90 (m, 4H), 2.05-2.20 (m, 4H), 2.80-2.90 (m, 8H), 3.40-3.60 (m, 6H), 3.75-3.80 (m, 1H), 5.30-5.45 (m, 10H), 6.90 (d, 1H), 7.25-7.35 (m, 1H), 7.45-7.55 (m, 1H), 7.80-7.95 (m, 2H), 10.56 (br s, 1H). MS (ESI): 596 [M+H]⁺, 618 [M+Na]⁺.

### Example 29

### Preparation of methyl 5-(2-ethyl-2-((5Z,8Z,11Z,14Z,17Z)-icosa-5,8,11,14,17-pentaen-1-ylthio)butanamido)-2-hydroxybenzoate

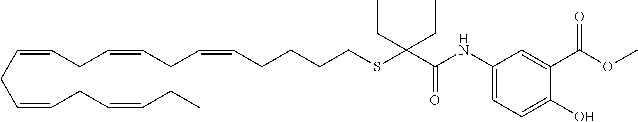 [SMILES: CC/C=C\C/C=C\C/C=C\C/C=C\C/C=C\CCCCSC(CC)(CC)C(=O)Nc1ccc(O)c(C(=O)OC)c1]

N-Methyl morpholine (NMM) (291 mg, 2.88 mmol) and TBTU (644 mg, 2.0 mmol) were added to a solution of 2-ethyl-2-((5Z,8Z,11Z,14Z,17Z)-icosa-5,8,11,14,17-pentaen-1-ylthio)butanoic acid (400 mg, 0.96 mmol) in DCM (5 ml). The reaction mixture was stirred for 15 minutes before methyl-5-aminosalicylate (319 mg, 1.91 mmol) was added. The mixture was stirred for 115 hours and then washed twice with water (2×5 mL). The organic layer was dried over Na₂SO₄, filtered and concentrated under reduced pressure. The residue was purified by preparative HPLC using a gradient of 10-0-10% water (containing 0.1% acetic acid) in MeCN (also containing 0.1% acetic acid) as eluent. The appropriate fractions were pooled and concentrated and the residue was dissolved in EtOAc. The solution was dried (Na₂SO₄) and concentrated to give 164 mg (30% yield) of the title compound. ¹H NMR (300 MHz, CDCl3): δ 10.64 (s, 1H), 8.90 (s, 1H), 8.15 (s, 1H), 7.60-7.47 (m, 1H), 7.06-6.92 (m, 1H), 5.38 (m, 10H), 3.96 (s, 3H), 2.97-2.72 (m, 8H), 2.56-2.36 (m, 2H), 2.16-2.01 (m, 4H), 2.01-1.87 (m, 2H), 1.87-1.70 (m, 2H), 1.69-1.55 (m, 2H), 1.55-1.40 (m, 2H), 1.04-0.92 (m, 9H).

### Example 30

### Preparation of 5-(2-ethyl-2-((5Z,8Z,11Z,14Z,17Z)-icosa-5,8,11,14,17-pentaen-1-ylthio)butanamido)-2-hydroxybenzoic acid

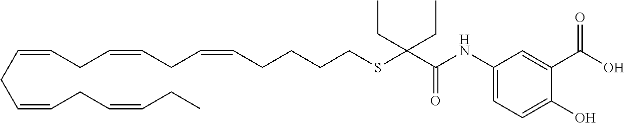 [SMILES: CC/C=C\C/C=C\C/C=C\C/C=C\C/C=C\CCCCSC(CC)(CC)C(=O)Nc1ccc(O)c(C(=O)O)c1]

2M NaOH (aq, 3 ml) was added to a solution of methyl 5-(2-ethyl-2-((5Z,8Z,11Z,14Z,17Z)-icosa-5,8,11,14,17-pentaen-1-ylthio)butanamido)-2-hydroxybenzoate (155 mg, 0.27 mmol) in MeOH (2 mL) and the reaction mixture was stirred at 50° C. for 24 hours. After cooling to room temperature, pH was adjusted to −2 with 5M HCl (aq). The resulting mixture was extracted twice with EtOAc (2×10 mL) and the combined organic extract was washed with brine (1×10 mL), dried (Na₂SO₄), filtered and concentrated. The residue was purified by preparative HPLC using a gradient of 20-0-20% water (containing 0.1% acetic acid) in MeCN (also containing 0.1% acetic acid) as eluent. The appropriate fractions were pooled and concentrated and the residue was dissolved in EtOAc. The solution was dried (Na₂SO₄) and concentrated to give 49 mg (33% yield) of the title compound. ¹H NMR (300 MHz, CDCl₃): δ 10.72-10.34 (m, 1H), 9.00 (s, 1H), 8.27-8.11 (m, 1H), 7.62-7.47 (m, 1H), 7.07-6.94 (m, 1H), 5.38 (m, 10H), 2.96-2.72 (m, 8H), 2.63-2.32 (m, 2H), 2.21-2.06 (m, 4H), 2.06-1.91 (m, 2H), 1.91-1.70 (m, 2H), 1.69-1.55 (m, 2H), 1.55-1.40 (m, 2H), 1.08-0.93 (m, 9H). MS (ESI): 576.4 [M+Na]⁺.

### Example 31

### Preparation of (S)-ethyl 2-(2-ethyl-2-((5Z,8Z,11Z,14Z,17Z)-icosa-5,8,11,14,17-pentaen-1-ylthio)butanamido)-4-methylpentanoate

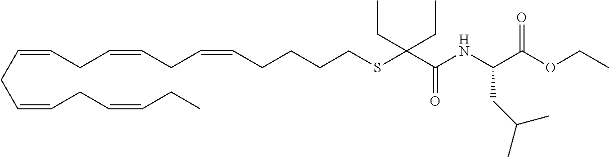 [SMILES: CC/C=C\C/C=C\C/C=C\C/C=C\C/C=C\CCCCSC(CC)(CC)C(=O)N[C@@H](CC(C)C)C(=O)OCC]

NMM (1.45 g, 14.3 mmol) and TBTU (3.1 g, 9.6 mmol) were added to a solution of 2-ethyl-2-((5Z,8Z,11Z,14Z,17Z)-icosa-5,8,11,14,17-pentaen-1-ylthio)butanoic acid (2.00 g, 4.78 mmol) in DCM (20 mL). The reaction mixture was stirred for 15 minutes before L-Leucine ethyl ester hydrochloride (0.94 g, 4.8 mmol) was added. The resulting mixture was stirred for 22 hours and then washed with water (10 mL), 1M HCl (aq, 10 mL), saturated NaHCO₃ (aq, 10 mL) and brine (10 mL). The organic phase was dried (Na₂SO₄), filtrated and concentrated to give 2.14 g (80% yield) of the title compound. ¹H NMR (300 MHz, CDCl3): δ 7.40 (t, 1H), 5.50-5.26 (m, 10H), 4.56 (m, 1H), 4.19 (q, 2H), 2.98-2.69 (m, 10H), 2.61-2.44 (m, 1H), 2.44-2.30 (m, 1H), 2.22-2.01 (m, 4H), 2.01-1.37 (m, 9H), 1.28 (t, 3H), 0.96 (m, 15H).

### Example 32

### Preparation of (S)-2-(2-ethyl-2-((5Z,8Z,11Z,14Z,17Z)-icosa-5,8,11,14,17-pentaen-1-ylthio)butanamido)-4-methylpentanoic acid

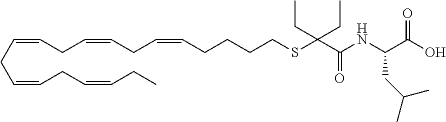 [SMILES: CC/C=C\C/C=C\C/C=C\C/C=C\C/C=C\CCCCSC(CC)(CC)C(=O)N[C@@H](CC(C)C)C(=O)O]

2M NaOH (aq, 15 mL) was added to a solution of (S)-ethyl 2-(2-ethyl-2-((5Z,8Z,11Z,14Z,17Z)-icosa-5,8,11,14,17-pentaen-1-ylthio)butanamido)-4-methylpentanoate (2.10 g, 3.8 mmol) in MeOH (10 mL) and the reaction mixture was stirred at 50° C. for 2 hours. After cooling to room temperature, pH was adjusted to −2 with 5M HCl (aq). The resulting mixture was extracted twice with EtOAc (2×10 mL) and the combined organic extract was dried (Na₂SO₄), filtered and concentrated. This afforded 1.65 g (83% yield) of the title compound. ¹H NMR (300 MHz, CDCl₃): δ 7.40 (d, 1H), 5.55-5.21 (m, 10H), 4.68-4.44 (m, 1H), 2.98-2.72 (m, 10H), 2.60-2.20 (m, 2H), 2.20-2.02 (m, 4H), 2.02-1.36 (m, 9H), 1.10-0.82 (m, 15H).

### Example 33

### Preparation of methyl 5-((S)-2-(2-ethyl-2-((5Z,8Z,11Z,14Z,17Z)-icosa-5,8,11,14,17-pentaen-1-ylthio)butanamido)-4-methylpentanamido)-2-hydroxybenzoate

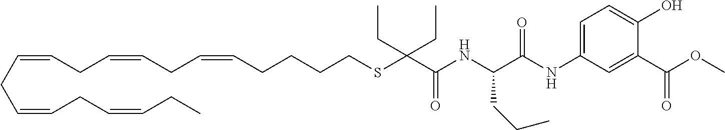 [SMILES: CC/C=C\C/C=C\C/C=C\C/C=C\C/C=C\CCCCSC(CC)(CC)C(=O)N[C@@H](CCC)C(=O)Nc1ccc(O)c(C(=O)OC)c1]

NMM (0.97 mL, 8.8 mmol) and TBTU (1.9 g, 5.9 mmol) were added to a solution of (S)-2-(2-ethyl-2-((5Z,8Z,11Z,14Z,17Z)-icosa-5,8,11,14,17-pentaen-1-ylthio)butanamido)-4-methylpentanoic acid (1.56 g, 2.93 mmol) in DCM (20 mL) at 0° C. The reaction mixture was stirred for 15 minutes at 0° C. before a solution of methyl-5-aminosalicylate (0.59 g, 3.5 mmol) in DCM (10 mL) was added. The resulting mixture was stirred at ambient temperature for 4 hours and then washed with water (10 mL), 1M HCl (aq, 10 mL), saturated NaHCO₃ (aq, 10 mL) and brine (10 mL). The organic phase was dried (Na₂SO₄), filtrated and concentrated to afford 1.67 g (85% yield) of the title compound. ¹H NMR (300 MHz, CDCl₃): δ 8.47 (s, 1H), 8.08 (d, 1H), 7.50 (dd, 1H), 7.34 (d, 1H), 7.06-6.86 (m, 1H), 5.55-5.17 (m, 10H), 4.65-4.42 (m, 1H), 3.95 (s, 3H), 3.68 (dd, 1H), 2.97-2.67 (m, 10H), 2.53-2.26 (m, 2H), 2.25-1.97 (m, 4H), 1.97-1.11 (m, 9H), 1.10-0.79 (m, 15H).

### Example 34

### Preparation 5-((S)-2-(2-ethyl-2-((5Z,8Z,11Z,14Z,17Z)-icosa-5,8,11,14,17-pentaen-1-ylthio)butanamido)-4-methylpentanamido)-2-hydroxybenzoic acid

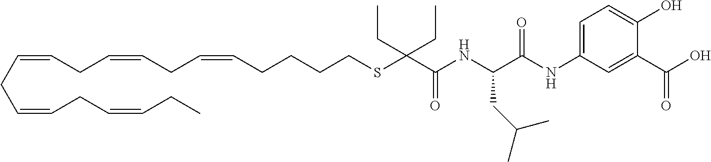 [SMILES: CC/C=C\C/C=C\C/C=C\C/C=C\C/C=C\CCCCSC(CC)(CC)C(=O)N[C@@H](CC(C)C)C(=O)Nc1ccc(O)c(C(=O)O)c1]

2M NaOH (aq, 9 mL) was added to a solution of methyl 5-((S)-2-(2-ethyl-2-((5Z,8Z,11Z,14Z,17Z)-icosa-5,8,11,14,17-pentaen-1-ylthio)butanamido)-4-methylpentanamido)-2-hydroxybenzoate (1.6 g, 2.3 mmol) in MeOH (6 mL) and the reaction mixture was stirred at 50° C. for 17 hours. After cooling to room temperature, pH was adjusted to ˜2 with 5M HCl (aq). The resulting mixture was extracted twice with EtOAc (2×15 mL) and the combined organic extract was dried (Na₂SO₄), filtered and concentrated. The residue was purified by preparative HPLC using a gradient of 30-0-30% water (containing 0.1% acetic acid) in MeCN (also containing 0.1% acetic acid) as eluent. The appropriate fractions were pooled and concentrated and the residue was dissolved in EtOAc. The solution was dried (Na₂SO₄) and concentrated to give 120 mg (8% yield) of the title compound. ¹H NMR (300 MHz, CDCl₃): δ 10.63-10.16 (m, 1H), 9.05-8.82 (m, 1H), 8.05-7.82 (m, 1H), 7.84-7.54 (m, 2H), 7.74-7.59 (m, 2H), 6.95-6.82 (m, 1H), 5.55-5.22 (m, 10H), 4.73-4.55 (m, 1H), 2.96-2.71 (m, 8H), 2.53-2.29 (m, 2H), 2.08 (dd, 4H), 1.77 (d, 7H), 1.66-1.50 (m, 1H), 1.50-1.33 (m, 1H), 0.99 (s, 15H). MS (ESI): 689.4 [M+Na]⁺.

1. A compound according to Formula I: 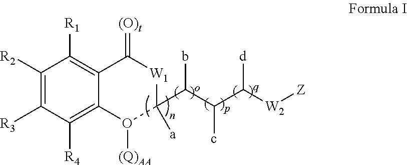
or a pharmaceutically acceptable salt, hydrate, solvate, prodrug, enantiomer, or stereoisomer thereof; wherein
R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;
W₁ and W₂ are each independently a bond, O, or —N(R)—, or when W₁ and W₂ are both NH, then both W₁ and W₂ can be taken together to form a piperidine moiety; [symbol]
represents an optional bond that when present requires that AA is 0;
a and c are each independently H, CH₃, —OCH₃, —OCH₂CH₃, or C(O)OH;
b is H, CH₃, C(O)OH, or O—Z;
d is H or C(O)OH;
each n, o, p, and q is independently 0 or 1;
each Z is independently H or 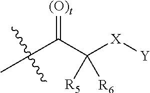
with the proviso that there is at least one 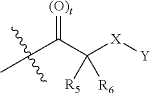
in the compound;
each t is independently 0 or 1;
each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂, wherein when X is CH₂, R₅ and R₆ are not both hydrogen;
Y is chosen from a C₁₀-C₂₄ alkyl, a C₁₀-C₂₄ alkenyl having 1-6 double bonds, and a C₁₀-C₂₂ alkynyl having 1-6 triple bonds;
Q is H, C(O)CH₃, Z, 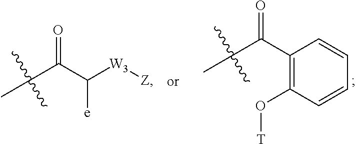
e is H or any one of the side chains of naturally occurring amino acids;
W₃ is a bond, —O—, or —N(R)—;
R is H or C₁-C₃ alkyl;
AA is 0 or 1; and
T is H, C(O)CH₃, or Z.

2. The compound according to claim 1, wherein the compound is present as a mixture of diastereomers, in racemic form, in the form of a diastereomer, or an enantiomer.

3. (canceled)

4. The compound according to claim 1, wherein Y is a C₁₀-C₂₂ alkenyl with 3-6 double bonds.

5. The compound according to claim 1, wherein Y is a C₁₀-C₂₂ alkenyl with 3-6 methylene interrupted double bonds in the Z configuration.

6. The compound according to claim 1, wherein R₅ and R₆ are chosen from a hydrogen atom, a methyl group, an ethyl group, a propyl group, a methoxy group, an ethoxy group, and an ethylthio group.

7. The compound according to claim 1, of formula Ia: 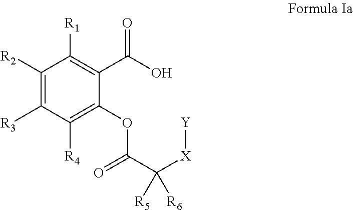
or a pharmaceutically acceptable salt, hydrate, solvate, prodrug, enantiomer, or stereoisomer thereof; wherein
R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;
each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

8. The compound according to claim 7, wherein the compound is present as a mixture of diastereomers, in racemic form, in the form of a diastereomer, or an enantiomer.

9. (canceled)

10. The compound according to claim 7, wherein Y is a C₁₀-C₂₂ alkenyl with 3-6 double bonds.

11. The compound according to claim 7, wherein Y is a C₁₀-C₂₂ alkenyl with 3-6 methylene interrupted double bonds in the Z configuration.

12. The compound according to claim 7, wherein R₅ and R₆ are chosen from a hydrogen atom, a methyl group, an ethyl group, a propyl group, a methoxy group, an ethoxy group, and an ethylthio group.

13-16. (canceled)

17. The compound according to claim 1, of Formula Ig: 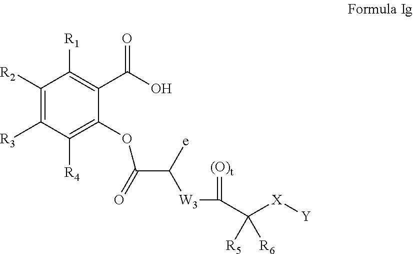
or a pharmaceutically acceptable salt, hydrate, solvate, prodrug, enantiomer, or stereoisomer thereof; wherein
R₁, R₂, R₃, and R₄ are each independently chosen from H, Cl, F, CN, NH₂, —NH(C₁-C₃ alkyl), —N(C₁-C₃ alkyl)₂, —NH(C(O)C₁-C₃ alkyl), —N(C(O)C₁-C₃ alkyl)₂, —C(O)H, —C(O)C₁-C₃ alkyl, —C(O)OC₁-C₃ alkyl, —C(O)NH₂, —C(O)NH(C₁-C₃ alkyl), —C(O)N(C₁-C₃ alkyl)₂, —C₁-C₃ alkyl, —O—C₁-C₃ alkyl, —S(O)C₁-C₃ alkyl, and —S(O)₂C₁-C₃ alkyl;
W₃ is a bond, O, or —N(R)—;
R is H or C₁-C₃ alkyl;
each t is independently 0 or 1;
each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds; and
e is H or any one of the side chains of naturally occurring amino acids.

18. The compound according to claim 17, wherein the compound is present as a mixture of diastereomers, in racemic form, in the form of a diastereomer, or an enantiomer.

19. (canceled)

20. The compound according to claim 17, wherein Y is a C₁₀-C₂₂ alkenyl with 3-6 double bonds.

21. The compound according to claim 17, wherein Y is a C₁₀-C₂₂ alkenyl with 3-6 methylene interrupted double bonds in the Z configuration.

22. The compound according to claim 17, wherein R₅ and R₆ are chosen from a hydrogen atom, a methyl group, an ethyl group, a propyl group, a methoxy group, an ethoxy group, and an ethylthio group.

23. The compound according to claim 1, of formula 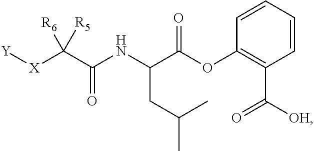
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

24. The compound according to claim 23, wherein the compound is present as a mixture of diastereomers, in racemic form, in the form of a diastereomer, or an enantiomer.

25. (canceled)

26. The compound according to claim 23, wherein Y is a C₁₀-C₂₂ alkenyl with 3-6 double bonds.

27. The compound according to claim 23, wherein Y is a C₁₀-C₂₂ alkenyl with 3-6 methylene interrupted double bonds in the Z configuration.

28. The compound according to claim 23, wherein R₅ and R₆ are chosen from a hydrogen atom, a methyl group, an ethyl group, a propyl group, a methoxy group, an ethoxy group, and an ethylthio group.

29-32. (canceled)

33. The compound according to claim 1, of formula 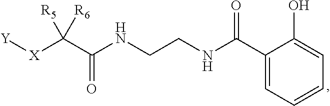
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

34. The compound according to claim 33, wherein the compound is present as a mixture of diastereomers, in racemic form, in the form of a diastereomer, or an enantiomer.

35. (canceled)

36. The compound according to claim 33, wherein Y is a C₁₀-C₂₂ alkenyl with 3-6 double bonds.

37. The compound according to claim 33, wherein Y is a C₁₀-C₂₂ alkenyl with 3-6 methylene interrupted double bonds in the Z configuration.

38. The compound according to claim 33, wherein R₅ and R₆ are chosen from a hydrogen atom, a methyl group, an ethyl group, a propyl group, a methoxy group, an ethoxy group, and an ethylthio group.

39-42. (canceled)

43. The compound according to claim 1, of formula 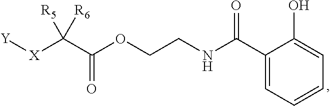
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

44. The compound according to claim 43, wherein the compound is present as a mixture of diastereomers, in racemic form, in the form of a diastereomer, or an enantiomer.

45. (canceled)

46. The compound according to claim 43, wherein Y is a C₁₀-C₂₂ alkenyl with 3-6 double bonds.

47. The compound according to claim 43, wherein Y is a C₁₀-C₂₂ alkenyl with 3-6 methylene interrupted double bonds in the Z configuration.

48. The compound according to claim 43, wherein R₅ and R₆ are chosen from a hydrogen atom, a methyl group, an ethyl group, a propyl group, a methoxy group, an ethoxy group, and an ethylthio group.

49-52. (canceled)

53. The compound according to claim 1, of formula 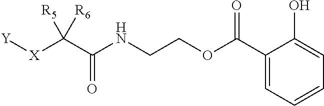
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

54. The compound according to claim 53, wherein the compound is present as a mixture of diastereomers, in racemic form, in the form of a diastereomer, or an enantiomer.

55. (canceled)

56. The compound according to claim 53, wherein Y is a C₁₀-C₂₂ alkenyl with 3-6 double bonds.

57. The compound according to claim 53, wherein Y is a C₁₀-C₂₂ alkenyl with 3-6 methylene interrupted double bonds in the Z configuration.

58. The compound according to claim 53, wherein R₅ and R₆ are chosen from a hydrogen atom, a methyl group, an ethyl group, a propyl group, a methoxy group, an ethoxy group, and an ethylthio group.

59-62. (canceled)

63. A compound according to Formula II: 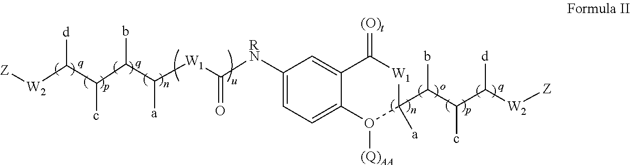
or a pharmaceutically acceptable salt, hydrate, solvate, prodrug, enantiomer, or stereoisomer thereof;
wherein each W₁ and W₂ are independently a bond, O, or —N(R)—, or when W₁ and W₂ are both NH, then both W₁ and W₂ can be taken together to form a piperidine moiety; [symbol]
represents an optional bond that when present requires that AA is 0;
each a and c are independently H, CH₃, —OCH₃, —OCH₂CH₃, or C(O)OH;
each b is H, CH₃, C(O)OH, or O—Z;
each d is H or C(O)OH;
each n, o, p, and q is independently 0 or 1;
each Z is H or 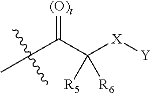
with the proviso that there is at least one 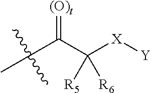
in the compound;
each t is independently 0 or 1;
each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂, wherein when X is CH₂, R₅ and R₆ are not both hydrogen;
Y is chosen from a C₁₀-C₂₄ alkyl, a C₁₀-C₂₄ alkenyl having 1-6 double bonds, and a C₁₀-C₂₂ alkynyl having 1-6 triple bonds;
u is 0 or 1;
Q is H, C(O)CH₃, Z, 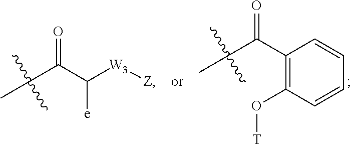
e is H or any one of the side chains of naturally occurring amino acids;
W₃ is a bond, —O—, or —N(R)—;
R is H or C₁-C₃ alkyl;
AA is 0 or 1; and
T is H, C(O)CH₃, or Z.

64. The compound of claim 63, of formula 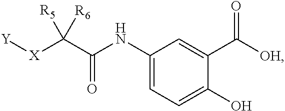
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

65. The compound according to claim 63, wherein the compound is present as a mixture of diastereomers, in racemic form, in the form of a diastereomer, or an enantiomer.

66. (canceled)

67. The compound according to claim 63, wherein Y is a C₁₀-C₂₂ alkenyl with 3-6 double bonds.

68. The compound according to claim 63, wherein Y is a C₁₀-C₂₂ alkenyl with 3-6 methylene interrupted double bonds in the Z configuration.

69. The compound according to claim 63, wherein R₅ and R₆ are chosen from a hydrogen atom, a methyl group, an ethyl group, a propyl group, a methoxy group, an ethoxy group, and an ethylthio group.

70-73. (canceled)

74. The compound according to claim 63, of the formula 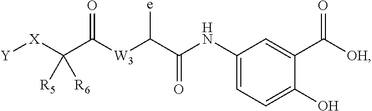
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds;
W₃ is a bond, —O—, or —N(R)—; and
e is H or any one of the side chains of naturally occurring amino acids.

75. The compound according to claim 74, wherein the compound is present as a mixture of diastereomers, in racemic form, in the form of a diastereomer, or an enantiomer.

76. (canceled)

77. The compound according to claim 74, wherein Y is a C₁₀-C₂₂ alkenyl with 3-6 double bonds.

78. The compound according to claim 74, wherein Y is a C₁₀-C₂₂ alkenyl with 3-6 methylene interrupted double bonds in the Z configuration.

79. The compound according to claim 74, wherein R₅ and R₆ are chosen from a hydrogen atom, a methyl group, an ethyl group, a propyl group, a methoxy group, an ethoxy group, and an ethylthio group.

80. The compound according to claim 74, of formula 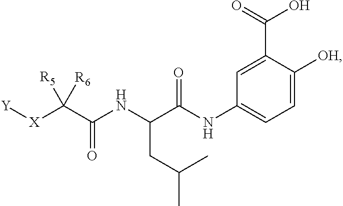
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

81-84. (canceled)

85. The compound according to claim 63, of formula 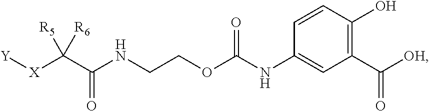
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

86. The compound according to claim 85, wherein the compound is present as a mixture of diastereomers, in racemic form, in the form of a diastereomer, or an enantiomer.

87. (canceled)

88. The compound according to claim 85, wherein Y is a C₁₀-C₂₂ alkenyl with 3-6 double bonds.

89. The compound according to claim 85, wherein Y is a C₁₀-C₂₂ alkenyl with 3-6 methylene interrupted double bonds in the Z configuration.

90. The compound according to claim 85, wherein R₅ and R₆ are chosen from a hydrogen atom, a methyl group, an ethyl group, a propyl group, a methoxy group, an ethoxy group, and an ethylthio group.

91-94. (canceled)

95. The compound according to claim 63, of formula 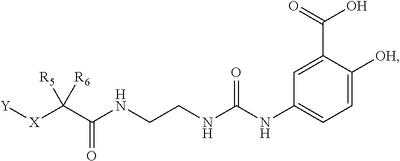
wherein each R₅ and R₆ is independently chosen from a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group;
X is chosen from O, S, SO, SO₂ and CH₂;
Y is chosen from a C₁₀-C₂₄ alkyl, a C₁₀-C₂₄ alkenyl having 1-6 double bonds, and a C₁₀-C₂₂ alkynyl having 1-6 triple bonds.

96. The compound according to claim 95, wherein the compound is present as a mixture of diastereomers, in racemic form, in the form of a diastereomer, or an enantiomer.

97. (canceled)

98. The compound according to claim 95, wherein Y is a C₁₀-C₂₂ alkenyl with 3-6 double bonds.

99. The compound according to claim 95, wherein Y is a C₁₀-C₂₂ alkenyl with 3-6 methylene interrupted double bonds in the Z configuration.

100. The compound according to claim 95, wherein R₅ and R₆ are chosen from a hydrogen atom, a methyl group, an ethyl group, a propyl group, a methoxy group, an ethoxy group, and an ethylthio group.

101-104. (canceled)

105. A method of preventing or treating at least one disease chosen from inflammation, rheumatoid arthritis, inflammatory bowel disease (IBD), atherosclerosis, diabetes, peripheral insulin resistance, dyslipidemia, metabolic syndrome comprising administering to a subject in need thereof at least one compound according to claim 1.

106-115. (canceled)

116. A method of lowering cholesterol comprising administering to a subject in need thereof at least one compound according to claim 1.

117-118. (canceled)

119. A method of raising HDL cholesterol comprising administering to a subject in need thereof at least one compound according to claim 1.

120. The compound according to claim 1, wherein Y is an omega-3 alkenyl.

121. A pharmaceutical composition comprising at least one compound according to claim 1 and a pharmaceutically acceptable carrier.

122-137. (canceled)

138. The compound according to claim 1 chosen from 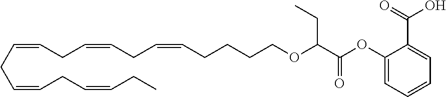 [SMILES: CC/C=C\C/C=C\C/C=C\C/C=C\C/C=C\CCCCOC(CC)C(=O)Oc1ccccc1C(=O)O]
2-((2-((5Z,8Z,11Z,14Z,17Z)-icosa-5,8,11,14,17-pentaen-1-yloxy)butanoyl)oxy)benzoic acid; 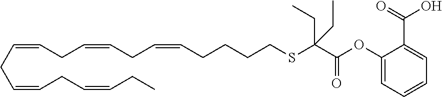 [SMILES: CC/C=C\C/C=C\C/C=C\C/C=C\C/C=C\CCCCSC(CC)(CC)C(=O)Oc1ccccc1C(=O)O]
2-((2-ethyl-2-((5Z,8Z,11Z,14Z,17Z)-icosa-5,8,11,14,17-pentaen-1-ylthio)butanoyl)oxy)benzoic acid; 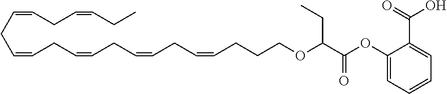 [SMILES: CC/C=C\C/C=C\C/C=C\C/C=C\C/C=C\C/C=C\CCCOC(CC)C(=O)Oc1ccccc1C(=O)O]
2-((2-((4Z,7Z,10Z,13Z,16Z,19Z)-docosa-4,7,10,13,16,19-hexaen-1-yloxy)butanoyl)oxy)benzoic acid; and 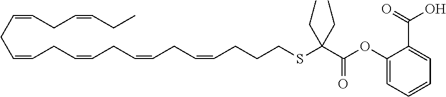 [SMILES: CC/C=C\C/C=C\C/C=C\C/C=C\C/C=C\C/C=C\CCCSC(CC)(CC)C(=O)Oc1ccccc1C(=O)O]
2-((2-((4Z,7Z,10Z,13Z,16Z,19Z)-docosa-4,7,10,13,16,19-hexaen-1-ylthio)-2-ethylbutanoyl)oxy)benzoic acid.

139. The compound according to claim 23 chosen from 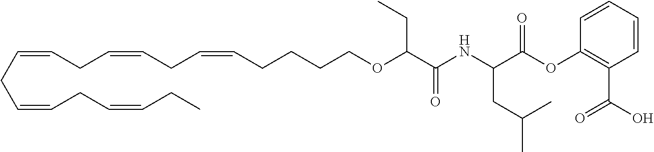 [SMILES: CC/C=C\C/C=C\C/C=C\C/C=C\C/C=C\CCCCOC(CC)C(=O)NC(CC(C)C)C(=O)Oc1ccccc1C(=O)O]
2-((2-(2-((5Z,8Z,11Z,14Z,17Z)-icosa-5,8,11,14,17-pentaen-1-yloxy)butanamido)-4-methylpentanoyl)oxy)benzoic acid; 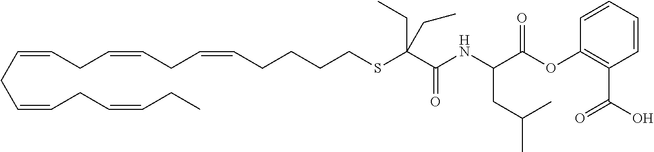 [SMILES: CC/C=C\C/C=C\C/C=C\C/C=C\C/C=C\CCCCSC(CC)(CC)C(=O)NC(CC(C)C)C(=O)Oc1ccccc1C(=O)O]
2-((2-(2-ethyl-2-((5Z,8Z,11Z,14Z,17Z)-icosa-5,8,11,14,17-pentaen-1-ylthio)butanamido)-4-methylpentanoyl)oxy)benzoic acid; 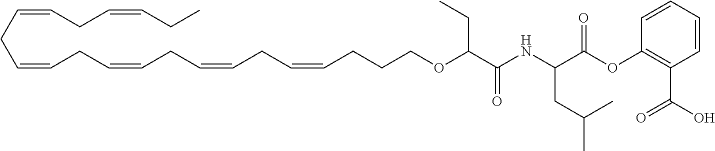 [SMILES: CC/C=C\C/C=C\C/C=C\C/C=C\C/C=C\C/C=C\CCCOC(CC)C(=O)NC(CC(C)C)C(=O)Oc1ccccc1C(=O)O]
2-((2-(2-((4Z,7Z,10Z,13Z,16Z,19Z)-docosa-4,7,10,13,16,19-hexaen-1-yloxy)butanamido)-4-methylpentanoyl)oxy)benzoic acid; and 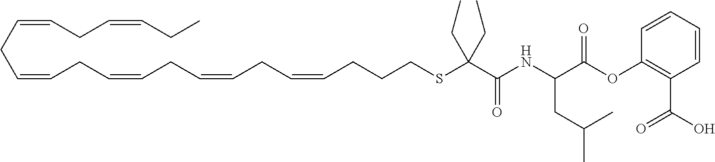 [SMILES: CC/C=C\C/C=C\C/C=C\C/C=C\C/C=C\C/C=C\CCCSC(CC)(CC)C(=O)NC(CC(C)C)C(=O)Oc1ccccc1C(=O)O]
2-((2-(2-((4Z,7Z,10Z,13Z,16Z,19Z)-docosa-4,7,10,13,16,19-hexaen-1-ylthio)-2-ethylbutanamido)-4-methylpentanoyl)oxy)benzoic acid.

140. The compound according to claim 33 chosen from 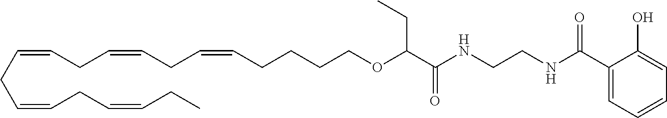 [SMILES: CC/C=C\C/C=C\C/C=C\C/C=C\C/C=C\CCCCOC(CC)C(=O)NCCNC(=O)c1ccccc1O]
2-hydroxy-N-(2-(2-((5Z,8Z,11Z,14Z,17Z)-icosa-5,8,11,14,17-pentaen-1-yloxy)butanamido)ethyl)benzamide; 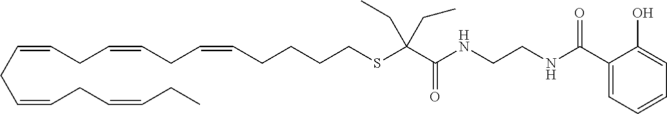 [SMILES: CC/C=C\C/C=C\C/C=C\C/C=C\C/C=C\CCCCSC(CC)(CC)C(=O)NCCNC(=O)c1ccccc1O]
N-(2-(2-ethyl-2-((5Z,8Z,11Z,14Z,17Z)-icosa-5,8,11,14,17-pentaen-1-ylthio)butanamido)ethyl)-2-hydroxybenzamide; 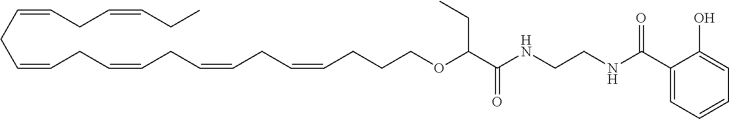 [SMILES: CC/C=C\C/C=C\C/C=C\C/C=C\C/C=C\C/C=C\CCCOC(CC)C(=O)NCCNC(=O)c1ccccc1O]
N-(2-(2-((4Z,7Z,10Z,13Z,16Z,19Z)-docosa-4,7,10,13,16,19-hexaen-1-yloxy)butanamido)ethyl)-2-hydroxybenzamide; and 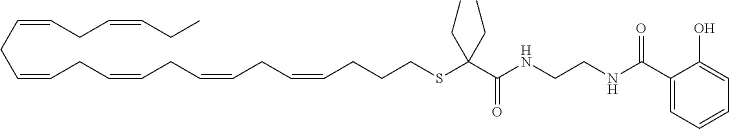 [SMILES: CC/C=C\C/C=C\C/C=C\C/C=C\C/C=C\C/C=C\CCCSC(CC)(CC)C(=O)NCCNC(=O)c1ccccc1O]
N-(2-(2-((4Z,7Z,10Z,13Z,16Z,19Z)-docosa-4,7,10,13,16,19-hexaen-1-ylthio)-2-ethylbutanamido)ethyl)-2-hydroxybenzamide.

141. The compound according to claim 43 chosen from 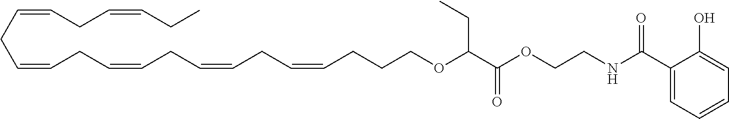 [SMILES: CC/C=C\C/C=C\C/C=C\C/C=C\C/C=C\C/C=C\CCCOC(CC)C(=O)OCCNC(=O)c1ccccc1O]
2-(2-hydroxybenzamido)ethyl 2-((4Z,7Z,10Z,13Z,16Z,19Z)-docosa-4,7,10,13,16,19-hexaen-1-yloxy)butanoate; 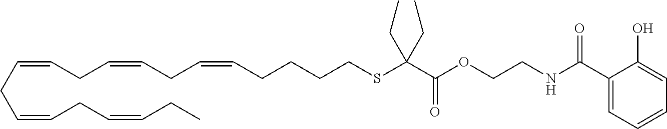 [SMILES: CC/C=C\C/C=C\C/C=C\C/C=C\C/C=C\CCCCSC(CC)(CC)C(=O)OCCNC(=O)c1ccccc1O]
2-(2-hydroxybenzamido)ethyl 2-ethyl-2-((5Z,8Z,11Z,14Z,17Z)-icosa-5,8,11,14,17-pentaen-1-ylthio)butanoate; 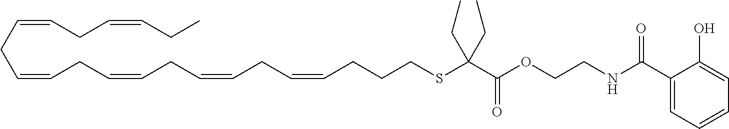 [SMILES: CC/C=C\C/C=C\C/C=C\C/C=C\C/C=C\C/C=C\CCCSC(CC)(CC)C(=O)OCCNC(=O)c1ccccc1O]
2-(2-hydroxybenzamido)ethyl 2-((4Z,7Z,10Z,13Z,16Z,19Z)-docosa-4,7,10,13,16,19-hexaen-1-ylthio)-2-ethylbutanoate; and 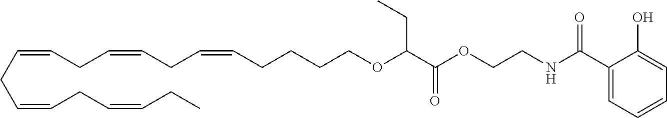 [SMILES: CC/C=C\C/C=C\C/C=C\C/C=C\C/C=C\CCCCOC(CC)C(=O)OCCNC(=O)c1ccccc1O]
2-(2-hydroxybenzamido)ethyl 2-((5Z,8Z,11Z,14Z,17Z)-icosa-5,8,11,14,17-pentaen-1-yloxy)butanoate.

142. The compound according to claim 53 chosen from 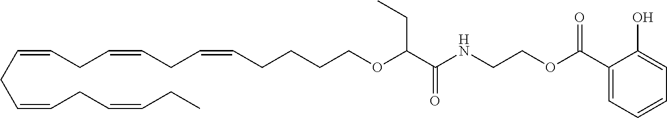 [SMILES: CC/C=C\C/C=C\C/C=C\C/C=C\C/C=C\CCCCOC(CC)C(=O)NCCOC(=O)c1ccccc1O]
2-(2-((5Z,8Z,11Z,14Z,17Z)-icosa-5,8,11,14,17-pentaen-1-yloxy)butanamido)ethyl 2-hydroxybenzoate; 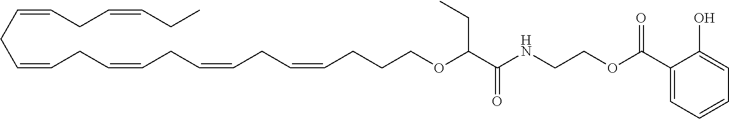 [SMILES: CC/C=C\C/C=C\C/C=C\C/C=C\C/C=C\C/C=C\CCCOC(CC)C(=O)NCCOC(=O)c1ccccc1O]
2-(2-((4Z,7Z,10Z,13Z,16Z,19Z)-docosa-4,7,10,13,16,19-hexaen-1-yloxy)butanamido)ethyl 2-hydroxybenzoate; 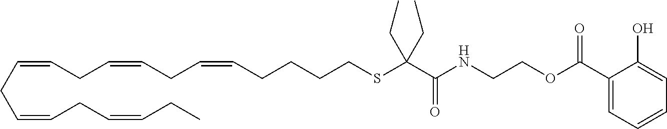 [SMILES: CC/C=C\C/C=C\C/C=C\C/C=C\C/C=C\CCCCSC(CC)(CC)C(=O)NCCOC(=O)c1ccccc1O]
2-(2-ethyl-2-((5Z,8Z,11Z,14Z,17Z)-icosa-5,8,11,14,17-pentaen-1-ylthio)butanamido)ethyl 2-hydroxybenzoate; and 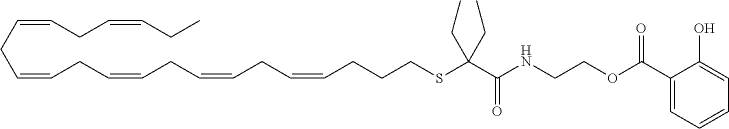 [SMILES: CC/C=C\C/C=C\C/C=C\C/C=C\C/C=C\C/C=C\CCCSC(CC)(CC)C(=O)NCCOC(=O)c1ccccc1O]
2-(2-((4Z,7Z,10Z,13Z,16Z,19Z)-docosa-4,7,10,13,16,19-hexaen-1-ylthio)-2-ethylbutanamido)ethyl 2-hydroxybenzoate.

143. The compound according to claim 64 chosen from 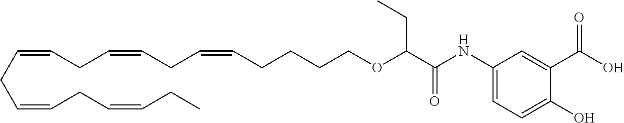 [SMILES: CC/C=C\C/C=C\C/C=C\C/C=C\C/C=C\CCCCOC(CC)C(=O)Nc1ccc(O)c(C(=O)O)c1]
2-hydroxy-5-(2-((5Z,8Z,11Z,14Z,17Z)-icosa-5,8,11,14,17-pentaen-1-yloxy)butanamido)benzoic acid; 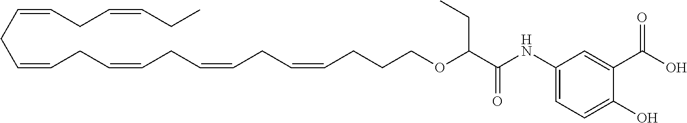 [SMILES: CC/C=C\C/C=C\C/C=C\C/C=C\C/C=C\C/C=C\CCCOC(CC)C(=O)Nc1ccc(O)c(C(=O)O)c1]
5-(2-((4Z,7Z,10Z,13Z,16Z,19Z)-docosa-4,7,10,13,16,19-hexaen-1-yloxy)butanamido)-2-hydroxybenzoic acid; 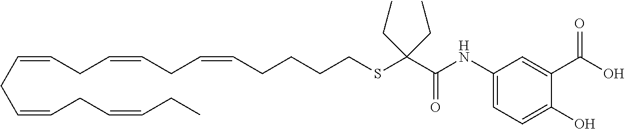 [SMILES: CC/C=C\C/C=C\C/C=C\C/C=C\C/C=C\CCCCSC(CC)(CC)C(=O)Nc1ccc(O)c(C(=O)O)c1]
5-(2-ethyl-2-((5Z,8Z,11Z,14Z,17Z)-icosa-5,8,11,14,17-pentaen-1-ylthio)butanamido)-2-hydroxybenzoic acid; and 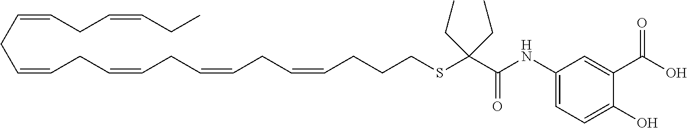 [SMILES: CC/C=C\C/C=C\C/C=C\C/C=C\C/C=C\C/C=C\CCCSC(CC)(CC)C(=O)Nc1ccc(O)c(C(=O)O)c1]
5-(2-((4Z,7Z,10Z,13Z,16Z,19Z)-docosa-4,7,10,13,16,19-hexaen-1-ylthio)-2-ethylbutanamido)-2-hydroxybenzoic acid.

144. The compound according to claim 74 chosen from 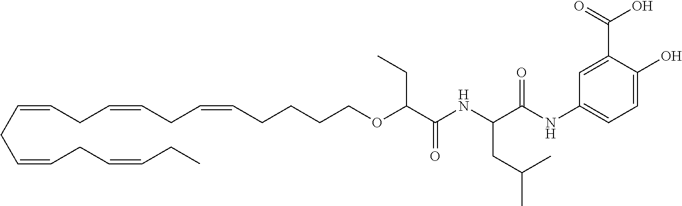 [SMILES: CC/C=C\C/C=C\C/C=C\C/C=C\C/C=C\CCCCOC(CC)C(=O)NC(CC(C)C)C(=O)Nc1ccc(O)c(C(=O)O)c1]
2-hydroxy-5-(2-(2-((5Z,8Z,11Z,14Z,17Z)-icosa-5,8,11,14,17-pentaen-1-yloxy)butanamido)-4-methylpentanamido)benzoic acid; 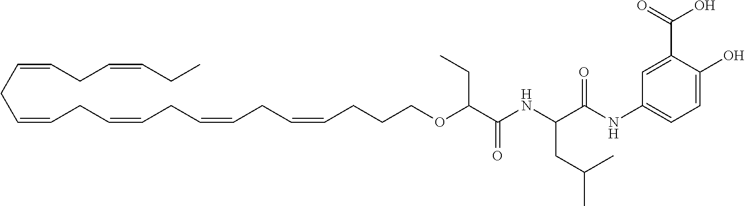 [SMILES: CC/C=C\C/C=C\C/C=C\C/C=C\C/C=C\C/C=C\CCCOC(CC)C(=O)NC(CC(C)C)C(=O)Nc1ccc(O)c(C(=O)O)c1]
5-(2-(2-((4Z,7Z,10Z,13Z,16Z,19Z)-docosa-4,7,10,13,16,19-hexaen-1-yloxy)butanamido)-4-methylpentanamido)-2-hydroxybenzoic acid; 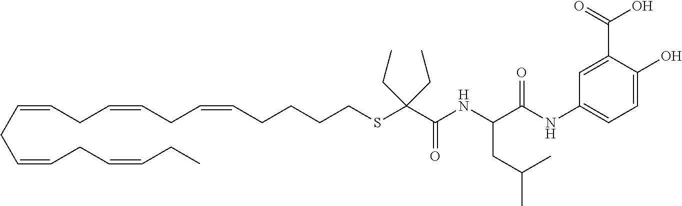 [SMILES: CC/C=C\C/C=C\C/C=C\C/C=C\C/C=C\CCCCSC(CC)(CC)C(=O)NC(CC(C)C)C(=O)Nc1ccc(O)c(C(=O)O)c1]
5-(2-(2-ethyl-2-((5Z,8Z,11Z,14Z,17Z)-icosa-5,8,11,14,17-pentaen-1-ylthio)butanamido)-4-methylpentanamido)-2-hydroxybenzoic acid; and 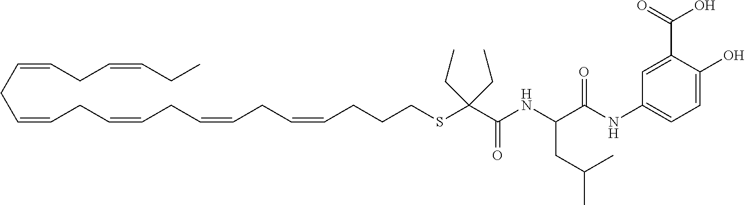 [SMILES: CC/C=C\C/C=C\C/C=C\C/C=C\C/C=C\C/C=C\CCCSC(CC)(CC)C(=O)NC(CC(C)C)C(=O)Nc1ccc(O)c(C(=O)O)c1]
5-(2-(2-((4Z,7Z,10Z,13Z,16Z,19Z)-docosa-4,7,10,13,16,19-hexaen-1-ylthio)-2-ethylbutanamido)-4-methylpentanamido)-2-hydroxybenzoic acid.

145. The compound according to claim 85 chosen from 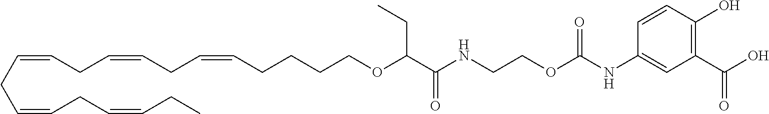 [SMILES: CC/C=C\C/C=C\C/C=C\C/C=C\C/C=C\CCCCOC(CC)C(=O)NCCOC(=O)Nc1ccc(O)c(C(=O)O)c1]
2-hydroxy-5-(((2-(2-((5Z,8Z,11Z,14Z,17Z)-icosa-5,8,11,14,17-pentaen-1-yloxy)butanamido)ethoxy)carbonyl)amino)benzoic acid; 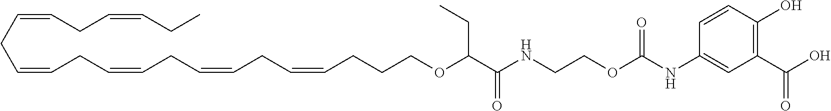 [SMILES: CC/C=C\C/C=C\C/C=C\C/C=C\C/C=C\C/C=C\CCCOC(CC)C(=O)NCCOC(=O)Nc1ccc(O)c(C(=O)O)c1]
5-(((2-(2-((4Z,7Z,10Z,13Z,16Z,19Z)-docosa-4,7,10,13,16,19-hexaen-1-yloxy)butanamido)ethoxy)carbonyl)amino)-2-hydroxybenzoic acid; 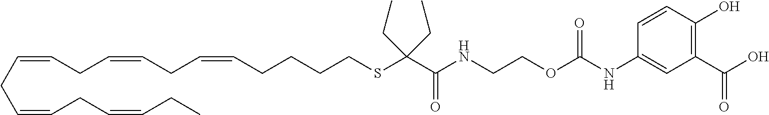 [SMILES: CC/C=C\C/C=C\C/C=C\C/C=C\C/C=C\CCCCSC(CC)(CC)C(=O)NCCOC(=O)Nc1ccc(O)c(C(=O)O)c1]
5-(((2-(2-ethyl-2-((5Z,8Z,11Z,14Z,17Z)-icosa-5,8,11,14,17-pentaen-1-ylthio)butanamido)ethoxy)carbonyl)amino)-2-hydroxybenzoic acid; and 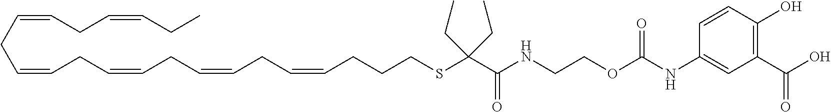 [SMILES: CC/C=C\C/C=C\C/C=C\C/C=C\C/C=C\C/C=C\CCCSC(CC)(CC)C(=O)NCCOC(=O)Nc1ccc(O)c(C(=O)O)c1]
5-(((2-(2-((4Z,7Z,10Z,13Z,16Z,19Z)-docosa-4,7,10,13,16,19-hexaen-1-ylthio)-2-ethylbutanamido)ethoxy)carbonyl)amino)-2-hydroxybenzoic acid.

146. The compound according to claim 95 chosen from 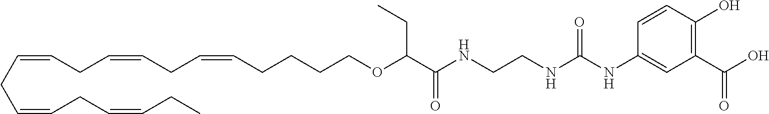 [SMILES: CC/C=C\C/C=C\C/C=C\C/C=C\C/C=C\CCCCOC(CC)C(=O)NCCNC(=O)Nc1ccc(O)c(C(=O)O)c1]
2-hydroxy-5-(3-(2-(2-((5Z,8Z,11Z,14Z,17Z)-icosa-5,8,11,14,17-pentaen-1-yloxy)butanamido)ethyl)ureido)benzoic acid; 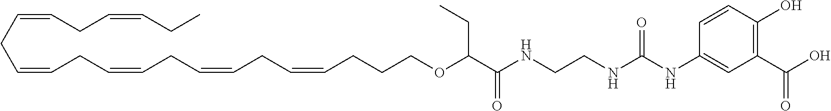 [SMILES: CC/C=C\C/C=C\C/C=C\C/C=C\C/C=C\C/C=C\CCCOC(CC)C(=O)NCCNC(=O)Nc1ccc(O)c(C(=O)O)c1]
5-(3-(2-(2-((4Z,7Z,10Z,13Z,16Z,19Z)-docosa-4,7,10,13,16,19-hexaen-1-yloxy)butanamido)ethyl)ureido)-2-hydroxybenzoic acid; 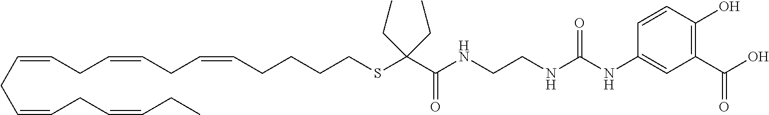 [SMILES: CC/C=C\C/C=C\C/C=C\C/C=C\C/C=C\CCCCSC(CC)(CC)C(=O)NCCNC(=O)Nc1ccc(O)c(C(=O)O)c1]
5-(3-(2-(2-ethyl-2-((5Z,8Z,11Z,14Z,17Z)-icosa-5,8,11,14,17-pentaen-1-ylthio)butanamido)ethyl)ureido)-2-hydroxybenzoic acid; and 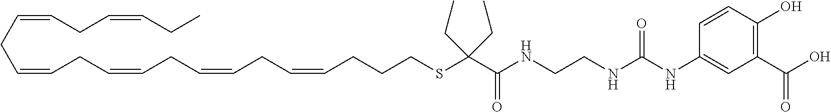 [SMILES: CC/C=C\C/C=C\C/C=C\C/C=C\C/C=C\C/C=C\CCCSC(CC)(CC)C(=O)NCCNC(=O)Nc1ccc(O)c(C(=O)O)c1]
5-(3-(2-(2-((4Z,7Z,10Z,13Z,16Z,19Z)-docosa-4,7,10,13,16,19-hexaen-1-ylthio)-2-ethylbutanamido)ethyl)ureido)-2-hydroxybenzoic acid.
